(19) United States
(12) Patent Application Publication
(10) Pub. No.: US 2024/0383867 A1
(43) Pub. Date: Nov. 21, 2024

(54) CRYSTALLINE FORMS OF ISOXAZOLINE COMPOUND

(71) Applicant: ELANCO US, INC., Greenfield, IN (US)

(72) Inventors: Lili HAN, Greenfield, IN (US); Qiaowen JIN, Greenfield, IN (US); Jennifer ROBIN, Greenfield, IN (US); Susan Margaret DE PAUL, Greenfield, IN (US); Guanmin WU, Greenfield, IN (US); Jingdan HU, Greenfield, IN (US); Stephen STIRM, Greenfield, IN (US)

(21) Appl. No.: 18/569,414

(22) PCT Filed: Jun. 15, 2022
(86) PCT No.: PCT/US22/33662

§ 371 Date: Dec. 12, 2023

(30) Foreign Application Priority Data
Jun. 16, 2021 (WO) PCT/CN2021/100305

(51) Int. Cl. C07D 261/04; A61K 31/42

(52) U.S. Cl. CPC C07D 261/04; A61K 31/42

(57) ABSTRACT

The present disclosure provides crystalline forms of 2-methyl-N-[2-oxo-2-(2,2,2-trifluoroethylamino)ethyl]-4-[(5S)-5-[3-chloro-2-fluoro-5-(trifluoromethyl)phenyl]-5-(trifluoromethyl)-4H-isoxazol-3-yl]benzamide, methods of preparing the same, and pharmaceutical compositions comprising the same.

### CROSS-REFERENCE TO RELATED PATENT APPLICATIONS

This patent application is an international patent application which claims priority to PCT No. PCT/CN2021/100305, filed on Jun. 16, 2021, the disclosure of each of which is incorporated herein in its entirety.

### TECHNICAL FIELD

The present invention relates to crystalline forms of an isoxazoline compound of Formula 1, methods of preparing the same, and pharmaceutical compositions including the same. More particularly, the present invention relates to crystalline forms of 2-methyl-N-[2-oxo-2-(2,2,2-trifluoroethylamino)ethyl]-4-[(5S)-5-[3-chloro-2-fluoro-5-(trifluoromethyl)phenyl]-5-(trifluoromethyl)-4H-isoxazol-3-yl]benzamide, methods of preparing the same, and pharmaceutical compositions including the same.

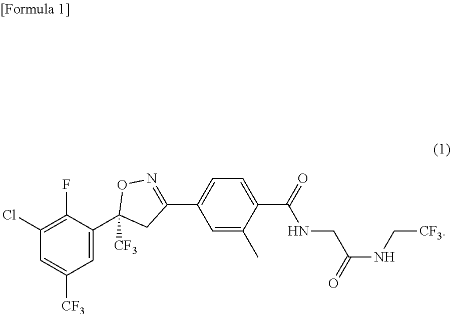 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

### BACKGROUND

U.S. patent application Ser. No. 17/125,365 (“US'365”) and international patent application no. PCT/US20/65624 (published as WO 2021/127188; “WO'188”) disclose 2-methyl-N-[2-oxo-2-(2,2,2-trifluoroethylamino)ethyl]-4-[(5S)-5-[3-chloro-2-fluoro-5-(trifluoromethyl)phenyl]-5-(trifluoromethyl)-4H-isoxazol-3-yl]benzamide of Formula 1 (“the compound of Formula 1”) and pharmaceutically acceptable salts thereof having an extended half-life in treating and controlling pests (in particular, fleas, ticks, mites, flies, worms, and lice) in animals (in particular, warm-blooded animals and fish).

US'365 and WO'188 also disclose a method of preparing the compound of Formula 1, as well as its R-enantiomer. The compound of Formula 1 produced by the methods of US'365 and WO'188 is amorphous. However, when trying to conduct a scale-up process based on the method disclosed in US'365 and WO'188, the compound of Formula 1 was obtained in an amorphous form, not crystalline solid. Such an amorphous form has lower purity and higher hygroscopicity compared to a crystalline solid, and is less desirable for commercial production.

### SUMMARY

One aspect of the present invention is to provide one or more crystalline forms of the compound of Formula 1, which have improved (i.e., reduced) hygroscopicity and improved purity and which is more suitable for mass (i.e., commercial or large-scale) production.

Another aspect is to provide a pharmaceutical composition containing one or more crystalline forms of the compound of Formula 1.

Another aspect is to provide a method of preparing one or more crystalline forms of the compound of Formula 1.

Other objectives and advantages of the present invention will become apparent from the following detailed descriptions along with the appended claims. Certain content not described in the present specification may be sufficiently recognized and inferred by those skilled in the art or similar fields of the present invention, and thus description thereof is omitted.

In one aspect of the present invention, there is provided a crystalline form A (also referred to as “form I”) of the compound of Formula 1, characterized in exhibiting an X-ray powder diffraction (XRPD) pattern comprising peaks at 2θ±0.2° values of 18.1°, 19.5°, and 22.3°.

In one aspect of the present invention, there is provided a crystalline form B (also referred to as “form II”) of the compound of Formula 1, characterized in exhibiting an X-ray powder diffraction (XRPD) pattern comprising peaks at 2θ±0.2° values of 3.5°, 19.2°, and 22.3°.

In one aspect of the present invention, there is provided a crystalline form C (also referred to as “form III”) of the compound of Formula 1, characterized in exhibiting an X-ray powder diffraction (XRPD) pattern comprising peaks at 2θ±0.2° values of 4.6°, 20.5°, and 21.7°.

In another aspect, there is provided a pharmaceutical composition comprising one or more crystalline forms of the compound of Formula 1 as active ingredient(s) and at least one pharmaceutically acceptable carrier or diluent.

In another aspect, there is provided a method of preparing a crystalline form A of the compound of Formula 1, the method comprising:
dissolving, optionally with stirring and/or heating, a compound of Formula 1 in component A, wherein component A is an organic solvent suitable for dissolving the compound of Formula 1, non-limiting examples of which include one or more of C₁-C₄ alcohol, acetone, acetonitrile, aniline, anisole, benzyl alcohol, butyronitrile, chloroform, cyclohexane, DMSO, ethanol, ethyl acetate, ethyl lactate, isopropyl acetate, heptane, n-heptane, isobutyl acetate, methyl ethyl ketone (MEK), methanol, medium-chain triglycerides (e.g., MIGLYOL® 812), N-methyl-2-pyrrolidone (NMP), 1-octanol, 1-propanol, 2-propanol, methyl tert-butyl ether (TBME), tetrahydrofuran (THF), toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, ethyl lactate, isopropyl acetate, isobutyl acetate, methyl ethyl ketone (MEK), methanol, medium-chain triglycerides (e.g., MIGLYOL® 812), N-methyl-2-pyrrolidone (NMP), 1-octanol, 1-propanol, 2-propanol, methyl tert-butyl ether (TBME), tetrahydrofuran (THF), toluene, and triethylamine;
optionally adding component B, wherein component B is an antisolvent that reduces the solubility of the mixture, wherein non-limiting examples of component B include one or more of water and C₅-C₁₂ cyclic or acyclic hydrocarbon alkanes (e.g., cyclohexane, hexane, heptane, n-heptane, octane, nonane, and decane), preferably one or more of water and heptane;
optionally evaporating the one or more components A and, if present, the one or more components B; and
filtering resulting solid.

In an aspect, the compound of Formula 1 dissolved in the dissolving step may be an amorphous form, a crystalline form, or a combination thereof. In another aspect, the compound of Formula 1 dissolved in the dissolving step is an amorphous form. In another aspect, component B (i.e., antisolvent) is present.

In another aspect, there is provided a method of preparing a crystalline form B of the compound of Formula 1, the method comprising:
dissolving, optionally with stirring and/or heating, a compound of Formula 1 in component A, wherein component A is an organic solvent suitable for dissolving the compound of Formula 1, non-limiting examples of which include one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine;
optionally adding component B, wherein component B is an antisolvent that reduces the solubility of the mixture, wherein non-limiting examples of component B include one or more of water and C₅-C₁₂ cyclic or acyclic hydrocarbon alkanes (e.g., cyclohexane, hexane, heptane, n-heptane, octane, nonane, and decane), preferably one or more of water and heptane;
evaporating the one or more components A and, if present, the one or more components B; and
filtering resulting solid.

In an aspect, the compound of Formula 1 dissolved in the dissolving step may be an amorphous form, a crystalline form, or a combination thereof. In another aspect, the compound of Formula 1 dissolved in the dissolving step is an amorphous form. In another aspect, component B (i.e., antisolvent) is present.

In another aspect, there is provided a method of preparing a crystalline form C of the compound of Formula 1, the method comprising:
dissolving, optionally with stirring and/or heating, a compound of Formula 1 in one or more component As, wherein component A is an organic solvent suitable for dissolving the compound of Formula 1, non-limiting examples of which include one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine;
adding one or more components B, wherein component B is an antisolvent that reduces solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane;
optionally evaporating the one or more components A and the one or more components B;
optionally adding additional component(s) B and evaporating; and filtering resulting solid.

In an aspect, the compound of Formula 1 dissolved in the dissolving step may be an amorphous form, a crystalline form, or a combination thereof. In another aspect, the compound of Formula 1 dissolved in the dissolving step is an amorphous form.

In another aspect, there is provided a crystalline form A, B or C of the compound of Formula 1 prepared by the above-described methods.

In another aspect of any of the above-described methods, component B (i.e., antisolvent) is present.

### BRIEF DESCRIPTION OF DRAWINGS

FIG. 1 shows an X-ray powder diffraction (XRPD) pattern of Sample 1 (an amorphous form of 2-methyl-N-[2-oxo-2-(2,2,2-trifluoroethylamino)ethyl]-4-[(5S)-5-[3-chloro-2-fluoro-5-(trifluoromethyl)phenyl]-5-(trifluoromethyl)-4H-isoxazol-3-yl]benzamide.

FIG. 2 shows a thermogram, produced by thermogravimetry coupled with Fourier-transform infrared spectroscopy (TG-FTIR), conducted on Sample 1 at a heating rate of 10° C./min up to 350° C.

FIG. 3 shows a differential scanning calorimetry (DSC) curve of Sample 1 with a heating rate of 10° C./min on up to 250° C.

FIG. 4 shows a dynamic vapor sorption (DVS) isotherm of Sample 1. Change in water content (thin curve) and relative humidity (thick curve) are shown as a function of time. The water content is calculated from the mass change of the sample during DVS measurement.

FIG. 5 shows a DVS isotherm of Sample 1. Change in water content is shown as a function of relative humidity. The water content is calculated from the mass change of the sample during DVS measurement.

FIG. 6 shows an overlay of XRPD patterns of Sample 1 before DVS (trace A, top) and after the DVS measurement (trace B, bottom). The diffractograms are offset in the y-direction for purposes of comparison.

FIG. 7 shows an overlay of XRPD patterns of Samples, from bottom to top, 2 (trace I), 3 (trace H), 4 (trace G), 5 (trace F), 6 (trace E), 7 (trace D), 11a-2 measured under a Kapton foil (trace C), 20 (trace B), and 12a measured under a Kapton foil (trace A); all of the samples in FIG. 7 are Form A. The broad peak at 5.6°2θ in some of the diffractograms is attributable to the Kapton foil. The diffractograms are offset in the y-direction for purposes of comparison.

FIG. 8 shows an overlay of XRPD patterns of Samples, from bottom to top, 8 (trace C), 10 (trace B), and 26 (trace A); all of the samples in FIG. 8 are Form B. The diffractograms are offset in the y-direction for purposes of comparison.

FIG. 9 shows an overlay of XRPD patterns of Samples, from bottom to top, 2 (trace B), and 10 (trace A). Sample 2 is Form A, and Sample 10 is Form B. The arrows point out the differences between the two XRPD patterns. The diffractograms are offset in the y-direction for purposes of comparison.

FIG. 10 shows an TG-FTIR thermogram conducted on Sample 2a (dried Form A).

FIG. 11 shows an TG-FTIR thermogram conducted on Sample 10a (dried Form B).

FIG. 12 shows an TG-FTIR thermogram conducted on Sample 13a (a mixture of Forms A and B).

FIG. 13 shows an XRPD pattern of Sample 2 (Form A).

FIG. 14 shows an overlay of XRPD patterns obtained during crystallization experiments. Sample numbers, from bottom to top, are 2 (trace P), 2a (trace O), 3 (trace N), 4 (trace M), 5 (trace L), 6 (trace K), 7 (trace J), 14 (trace 1), 15 (trace H), 17 (trace G), 18 (trace F), 19 (trace E), 20 (trace D), 21 (trace C), 23 (trace B), and 28 (trace A). The broad reflection at approximately 5.6°2θ in the Sample 23 corresponds to the signal of the Kapton foil used for the measurement of wet Sample 23. The diffractograms are offset in the y-direction for purposes of comparison.

FIG. 15 shows a DSC curve of the dried Form A Sample 2a.

FIG. 16 shows a DVS isotherm of Sample 2a. Change in water content (thin curve) and relative humidity (thick curve) are shown as a function of time. The water content is calculated from the mass change of the sample during DVS measurement.

FIG. 17 shows a DVS isotherm of Sample 2a. Change in water content is shown as a function of relative humidity. The water content is calculated from the mass change of the sample during DVS measurement.

FIG. 18 shows an overlay of XRPD patterns of Sample 2a (Form A) before DVS (trace B, bottom) and after the DVS measurement (trace A, top). The diffractograms are offset in the y-direction for purposes of comparison.

FIG. 19 shows an XRPD pattern of Sample 10 (Form B).

FIG. 20 shows a DSC curve of the dried Form B Sample 10a.

FIG. 21 shows a DSC curve of the dried Form B Sample 30a.

FIG. 22 shows a DVS isotherm of Sample 10 (Form B). Change in water content (thin curve) and relative humidity (thick curve) are shown as a function of time. The water content is calculated from the mass change of the sample during DVS measurement.

FIG. 23 shows a DVS isotherm of Sample 10 (Form B). Change in water content is shown as a function of relative humidity. The water content is calculated from the mass change of the sample during DVS measurement.

FIG. 24 shows an overlay of XRPD patterns of Sample 10 (Form A) before DVS (trace B, bottom) and after the DVS measurement (trace A, top). The diffractograms are offset in the y-direction for purposes of comparison.

FIG. 25 shows an overlay of XRPD patterns obtained during mechanical stress experiments; measurements were taken using a D8 Advance (Bruker ASX, Germany) analyzer. Sample numbers, from bottom to top, are 37 (trace D, Form A reference), 38 (trace C, Form A ground), 39 (trace B, Form A ball milled), and 40 (trace A, Form A pressed at 15 bars). The diffractograms are offset in the y-direction for purposes of comparison.

FIG. 26 shows an overlay of XRPD patterns obtained during mechanical stress experiments; measurements were taken using a D8 Advance (Bruker ASX, Germany) analyzer. Sample numbers are 37 (trace B, Form A reference) and 38 (trace A, Form A ground).

FIG. 27 shows an overlay of XRPD patterns obtained during mechanical stress experiments; measurements were taken using a D8 Advance (Bruker ASX, Germany) analyzer. Sample numbers are 37 (trace B, Form A reference) and 39 (trace A, Form A ball milled).

FIG. 28 shows an overlay of XRPD patterns obtained during mechanical stress experiments; measurements were taken using a D8 Advance (Bruker ASX, Germany) analyzer. Sample numbers are 37 (trace B, Form A reference) and 40 (trace A, Form A after pressing at 15 bars).

FIG. 29 shows an XRPD pattern of Sample 2a (Form A), with peak picking.

FIG. 30 shows an XRPD pattern of Sample 10a (Form B), with peak picking.

FIG. 31 shows solubility of Form A in ethyl acetate/heptane 1:3 (curve A), in 2-propanol/water 1:1 (curve B), and in TBME/heptane 1:1 (curve C) as a function of temperature.

FIG. 32 shows a plot of the natural logarithm of the solubility of Form A in ethyl acetate/heptane 1:3 versus the inverse of the temperature (in Kelvin).

FIG. 33 shows a plot of the natural logarithm of the solubility of Form A in 2-propanol/water 1:1 versus the inverse of the temperature (in Kelvin).

FIG. 34 shows a plot of the natural logarithm of the solubility of Form A in TBME/heptane 1:1 versus the inverse of the temperature (in Kelvin).

FIG. 35 shows light microscopy images from Sample 43 (Form A+a small amount of Form B): powder as-is (left panel) and powder suspended in paraffin oil (right panel).

FIG. 36 shows light microscopy images from Sample 44 (Form A): powder as-is (left panel) and powder suspended in paraffin oil (right panel).

FIG. 37 shows light microscopy images from Sample 45 (Form A): powder as-is (left panel) and powder suspended in paraffin oil (right panel).

FIG. 38 shows light microscopy images from Sample 46 (Form A): powder as-is (left panel) and powder suspended in paraffin oil (right panel).

FIG. 39 shows light microscopy images from Sample 47 (Form A): powder as-is (left panel) and powder suspended in paraffin oil (right panel).

FIG. 40 shows light microscopy images from Sample 48 (Form A): powder as-is (left panel) and powder suspended in paraffin oil (right panel).

FIG. 41 shows light microscopy images from Sample 49 (Form A): powder as-is (left panel) and powder suspended in paraffin oil (right panel).

FIG. 42 shows light microscopy images from Sample 50 (Form A): powder as-is (left panel) and powder suspended in paraffin oil (right panel).

FIG. 43 shows light microscopy images from Sample 51 (Form A): powder as-is (left panel) and powder suspended in paraffin oil (right panel).

FIG. 44 shows light microscopy images from Sample 52 (Form A): powder as-is (left panel) and powder suspended in paraffin oil (right panel).

FIG. 45 shows light microscopy images from Sample 42 (Form A), used for seeding in small-scale experiments: powder as-is (left panel) and powder suspended in paraffin oil (right panel).

FIG. 46 shows an overlay of XRPD patterns of Samples, from bottom to top, 43 (trace C, mixture of Forms A and B), 42 (trace B, Form A), and 10 (trace A, Form B). The arrows show the Form B peaks which are observed in the diffractogram of Sample 43. The diffractograms are offset in the y-direction for purposes of comparison.

FIG. 47 shows an overlay of XRPD patterns of Samples, from bottom to top, 42 (trace K), 43 (trace J), 44 (trace 1), 45 (trace H), 46 (trace G), 47 (trace F), 48 (trace E), 49 (trace D), 50 (trace C), 51 (trace B), and 52 (trace A); all of the samples in FIG. 47 are Form A, except Sample 43, which also contains a small amount of Form B. The diffractograms are offset in the y-direction for purposes of comparison.

FIG. 48 shows a graphical representation of experiment 48: the temperature (curve B) and the turbidity (curve A) are shown as a function of time. The marker at curve C represents the seeding point.

FIG. 49 shows a graphical representation of experiments 50 and 51: the temperature (curve A) and the turbidity (curve C for experiment 50 and curve B for experiment 51) are shown as a function of time. The markers on the x-axis represent the seeding points.

FIG. 50 shows a graphical representation of experiment 53: the temperature (curve D), water volume (curve A), counts of chords from 100 to 1000 μm (curve E), counts of chords from 10 to 100 μm (curve B), and counts of chord length <10 μm (curve C) are shown as functions of time. The data recording was started when the reactor was already at 60° C.

FIG. 51 shows a graphical representation of data collected with Particle Track G400 Probe for experiment 53: counts of chords from 100 to 1000 μm (curve C), counts of chords from 10 to 100 μm (curve A), counts of chord length <10 μm (curve B), and the mean square (curve D) are shown as functions of time.

FIG. 52 shows an XRPD pattern of Sample 53.

FIG. 53 shows an overlay of XRPD patterns of Samples, from bottom to top, 53 (trace C), 42 (trace B, Form A), and 10 (trace A, Form B). The diffractograms are offset in the y-direction for purposes of comparison.

FIG. 54 shows light microscopy images from Sample 53: powder as-is (left panel) and powder suspended in paraffin oil (right panel).

FIG. 55 shows an TG-FTIR thermogram conducted on Sample 53.

FIG. 56 shows a proton nuclear magnetic resonance (¹H-NMR) spectrum of Sample 53 recorded in DMSO-d₆. The peak at 3.3 ppm corresponds to the water contained in the DMSO-d₆ solvent, and the peak at 2.5 ppm corresponds to the DMSO-d₆ solvent.

FIG. 57 shows a ¹H-NMR spectrum of the amorphous form of the compound of Formula 1 recorded in DMSO-d₆. The peak at 3.3 ppm corresponds to the water contained in the DMSO-d₆ solvent, and the peak at 2.5 ppm corresponds to the DMSO-d₆ solvent.

FIG. 58 shows high-performance liquid chromatography (HPLC) results for Sample 53: the whole HPLC chromatogram (top panel), the zoomed-in region corresponding to retention time of 5.9 to 7.1 minutes (middle panel), and a summary table of the detected peaks (bottom panel).

FIG. 59 shows a graphical representation of data collected with the EasyViewer probe for experiment 54: counts of in-focus particles with length in the range of 100-1000 μm (curve E), counts of in-focus particles with length in the range of 10-100 μm (curve C), counts of in-focus particles with length <10 μm (curve F), turbidity (curve B), temperature (dotted curve D), and the added volume (dotted curve A) are shown as functions of time.

FIG. 60 shows particles observed with the EasyViewer probe in experiment 54 after seeding with Sample 46 (Form A).

FIG. 61 shows particles observed with the EasyViewer probe in experiment 54 after starting of the first cooling.

FIG. 62 shows particles observed with the EasyViewer probe at the end of experiment 54, at 20° C.

FIG. 63 shows an XRPD pattern of Sample 54 (Form A).

FIG. 64 shows an overlay of XRPD patterns of Samples, from bottom to top, 54 (trace C), 42 (trace B, Form A), and 10 (trace A, Form B). The diffractograms are offset in the y-direction for purposes of comparison.

FIG. 65 shows light microscopy images from Sample 54: powder as-is (left panel) and powder suspended in paraffin oil (right panel).

FIG. 66 shows an TG-FTIR thermogram conducted on Sample 54.

FIG. 67 shows a ¹H-NMR spectrum of Sample 54 recorded in DMSO-d₆. The peak at 3.3 ppm corresponds to the water contained in the DMSO-d₆ solvent, and the peak at 2.5 ppm correspond to the DMSO-d₆ solvent.

FIG. 68 shows HPLC results for Sample 54: the whole HPLC chromatogram (top panel), the zoomed-in region corresponding to retention time of 5.9 to 7.1 minutes (middle panel), and a summary table of the detected peaks (bottom panel).

FIG. 69 shows an XRPD pattern of Sample 55 (Form C).

FIG. 70 shows an overlay of XRPD patterns of Samples, from bottom to top, 2 (trace A, Form A), 10 (trace B, Form B), and 55 (trace C, Form C). The diffractograms are offset in the y-direction for purposes of comparison.

FIG. 71 shows a DSC curve of the Form C Sample 55.

FIG. 72 shows scanning electron microscope (SEM) images of Sample 55. Magnification is 100× in the left panel and 250× in the right panel.

FIG. 73 shows SEM images of Sample 55. Magnification is 500× in the left panel and 1000× in the right panel.

FIG. 74 shows SEM images of Sample 55. Magnification is 3000× in the left panel and 9000× in the right panel.

FIG. 75 shows an overlay of XRPD patterns of Samples, from bottom to top, 63 (trace D), 63A (trace C), 42 (trace B, Form A), and 55 (trace A, Form C). The diffractograms are offset in the y-direction for purposes of comparison. The arrows point to Form A reflections.

FIG. 76 shows an overlay of XRPD patterns of Samples, from bottom to top, 64 (trace E), 64A (trace D), 64B (trace C), 42 (trace B, Form A), and 55 (trace A, Form C). The diffractograms are offset in the y-direction for purposes of comparison. The arrow points to a Form A reflection.

### DETAILED DESCRIPTION OF THE INVENTION

Hereinafter, the present invention will be described in greater detail. Unless otherwise defined, all technical terms used in the present invention have the same meaning as commonly understood by those skilled in the related art of the present invention.

The contents of all publications cited as reference documents herein are incorporated in the present specification by reference in their entirety.

Descriptions and embodiments disclosed in one portion of the present invention may be applied to other descriptions and embodiments in other portions of the present invention. That is, all combinations of various elements disclosed in the present invention belong within the scope of the present invention. Additionally, the scope of the present invention should not be limited by the specific descriptions described herein below.

In addition, although a preferred method or sample is described in the specification, those similar or equivalent thereto fall within the scope of the present invention. In addition, the term “comprising” is intended to have an open-ended meaning and permits the inclusion of additional elements that are not identified.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to being within 5% of a particular value or range, and more preferably within 1% to 2%. For example, “about 10%” refers to 9.5% to 10.5%, and preferably, 9.8% to 10.2%. For another example, “about 100° C.” refers to 95° C. to 105° C., and preferably, 98° C. to 102° C.

Unless otherwise indicated, all XRPD measurements in the instant disclosure are taken at wavelength 1.5405958 Å using (STOE STADI P) Cu-Kα1 monochromator. Exceptions include Samples 37-40 and 56-60, described herein, and post-solubility testing of Samples 42 and 55, also described herein; these exceptions were measured at wavelength 1.5405958 Å using Cu-Kα D8 Advance (Bruker ASX, Germany) analyzer.

Unless otherwise specified, it should be apparent to a skilled person in the art that the values of peaks from X-ray powder diffraction studies reported in this application are associated with experimental errors typically observable in this field. Specifically, a peak is interpreted to be located within the angle variation ±0.5° of the value reported herein. Preferably, a peak is interpreted to be located within the angle variation ±0.2° of the value reported herein, more preferably, within the angle variation ±0.1°.

The S-enantiomer is believed to be more active than the R-enantiomer. Therefore, the S-enantiomer is preferred.

### Amorphous Form of Compound of Formula 1

Properties of the amorphous form of the compound of Formula 1, as disclosed herein and in US'365 and WO'188 are described below.

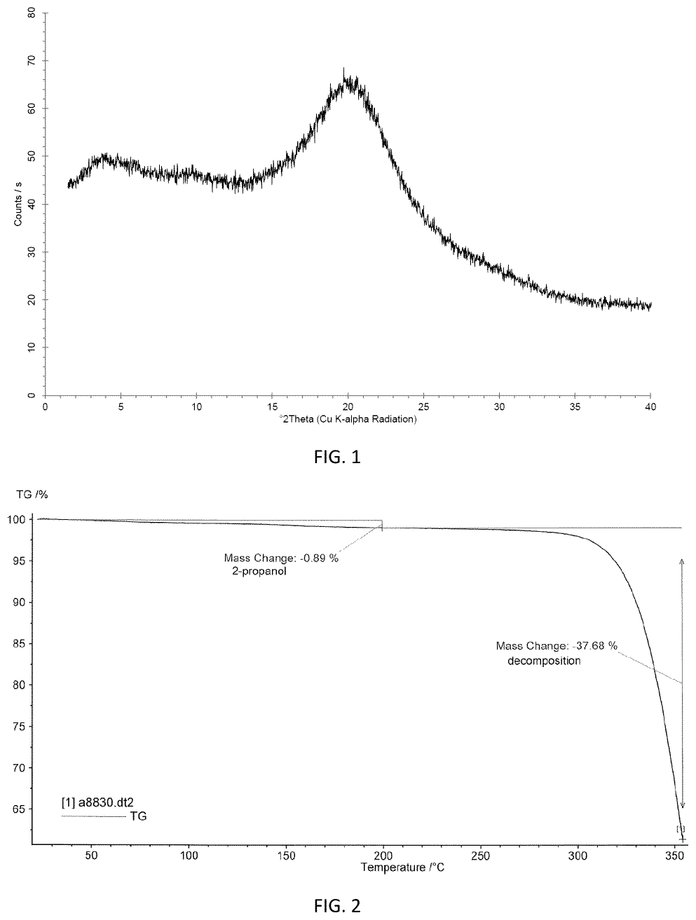

An X-ray powder diffraction (XRPD) of the amorphous form of the compound of Formula 1 is set forth in FIG. 1.

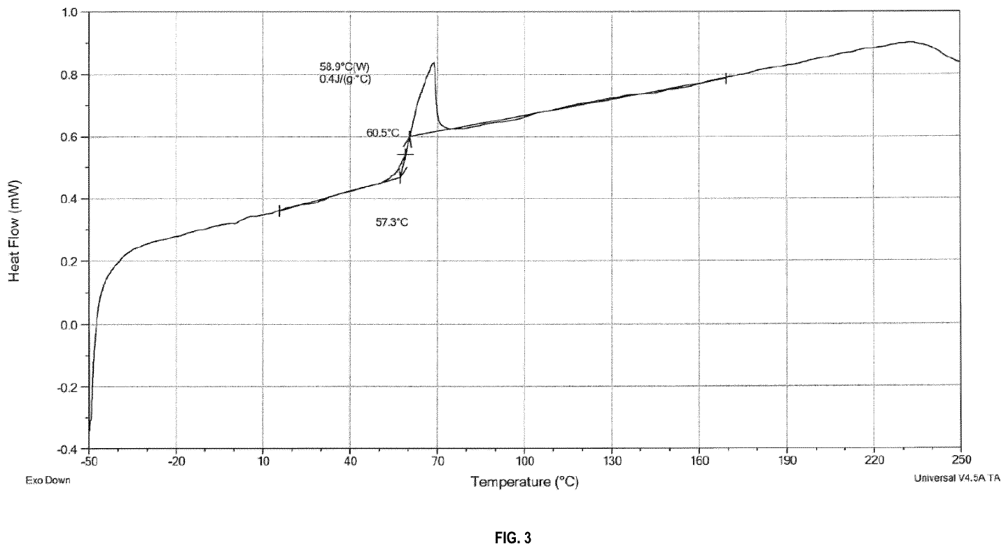

The amorphous form of a sample of the compound of Formula 1, when subjected to differential scanning calorimetry (“DSC”), exhibited a glass transition at 59° C., with a ΔCₚ step of 0.4 J/(g·° C.) (see FIG. 3).

The amorphous form of a sample of the compound of Formula 1, which sample contained residual amounts of isopropanol, may have, in a thermogravimetric analysis (TG-FTIR), a weight loss of about 0.9% at the temperature of up to about 200° C. that corresponds to isopropanol, and may decompose at or above 280° C. (see FIG. 2).

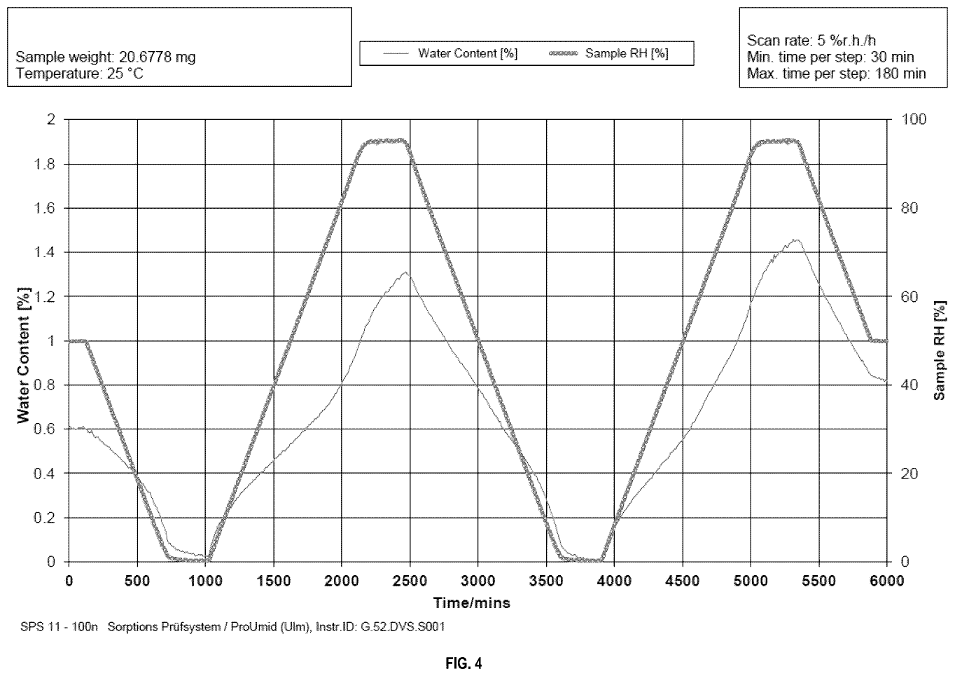

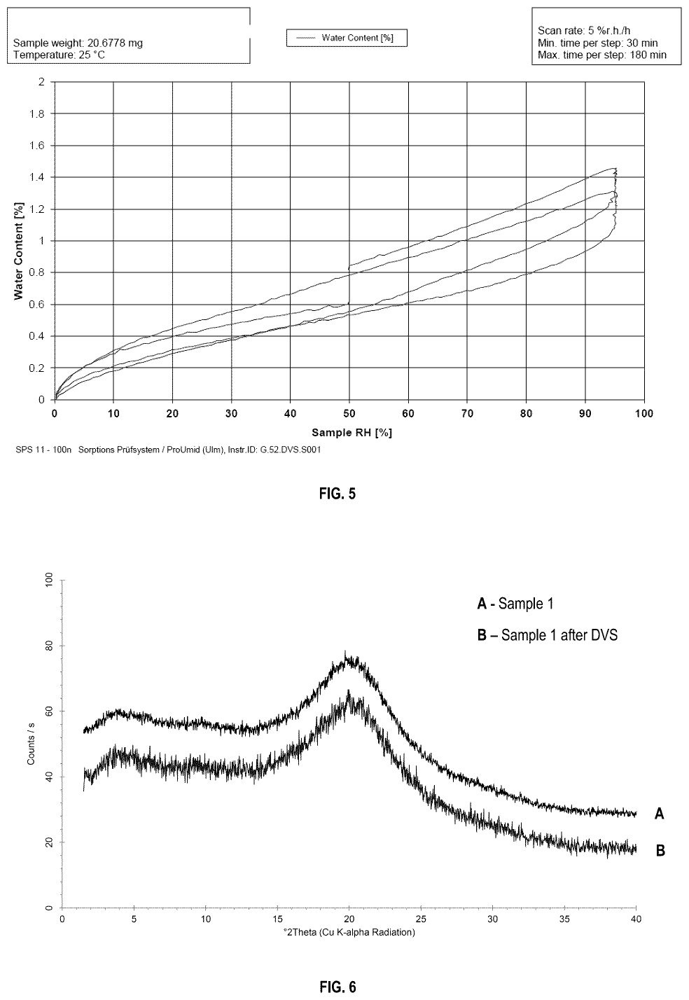

According to hygroscopicity testing (via dynamic vapor sorption, or DVS), the amorphous form of the compound of Formula 1 is slightly hygroscopic, absorbing almost 1.5% of water upon storage at 95% relative humidity for five hours (see FIGS. 4 and 5). No crystallization occurred during DVS testing of the amorphous form of the compound of Formula 1; the amorphous form is kinetically fairly stable (see FIG. 6).

### Crystalline Forms of Compound of Formula 1

### Form A

One aspect of the present disclosure provides a crystalline form of the compound of Formula 1, characterized in exhibiting an XRPD pattern comprising peaks at diffraction angles 2θ±0.2° values of 18.1°, 19.5°, and 22.3°. Hereinafter, this crystalline form is referred to as Crystalline Form A.

In one aspect, Crystalline Form A may exhibit an XRPD pattern comprising peaks at three or more, four or more, five or more, six or more, seven or more, eight or more, nine or more, ten or more, eleven or more, twelve or more, thirteen or more, fourteen or more, fifteen or more, preferably four or more, 2θ±0.2° values selected from the group consisting of 3.5°, 7.1°, 8.9°, 9.1°, 9.6°, 10.7°, 12.6°, 14.0°, 16.9°, 18.1°, 18.4°, 18.9°, 19.5°, 20.1°, 21.0°, 22.0°, 22.3°, and 22.7°.

In another aspect, Crystalline Form A may exhibit an XRPD pattern comprising peaks at three or more, four or more, five or more, six or more, seven or more, eight or more, nine or more, ten or more, eleven or more, twelve or more, thirteen or more, fourteen or more, fifteen or more, preferably four or more, 2θ±0.2° values selected from the group consisting of 3.53°, 7.10°, 8.94°, 9.12°, 9.63°, 10.68°, 12.63°, 13.95°, 16.86°, 18.06°, 18.39°, 18.90°, 19.48°, 20.10°, 21.00°, 22.00°, 22.26°, and 22.71°.

In particular, Crystalline Form A may exhibit an XRPD pattern comprising peaks at 2θ±0.2° values of 3.5°, 7.1°, 8.9°, 9.1°, 9.6°, 10.7°, 18.1°, 18.4°, 18.9°, 19.5°, 20.1°, 21.0°, 22.0°, 22.3°, and 22.7°.

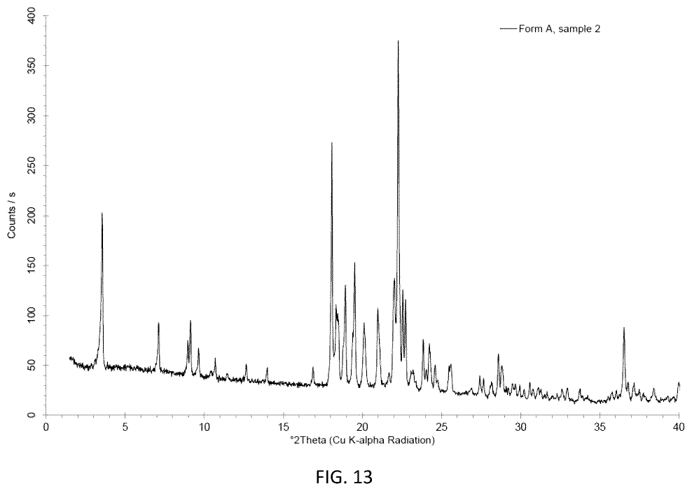

In more particularity, Crystalline Form A may exhibit an XRPD pattern comprising peaks at 2θ±0.2° values of 3.5°, 7.1°, 8.9°, 9.1°, 9.6°, 10.7°, 12.6°, 14.0°, 16.9°, 18.1°, 18.4°, 18.9°, 19.5°, 20.1°, 21.0°, 22.0°, 22.3°, and 22.7°. (See FIG. 13.)

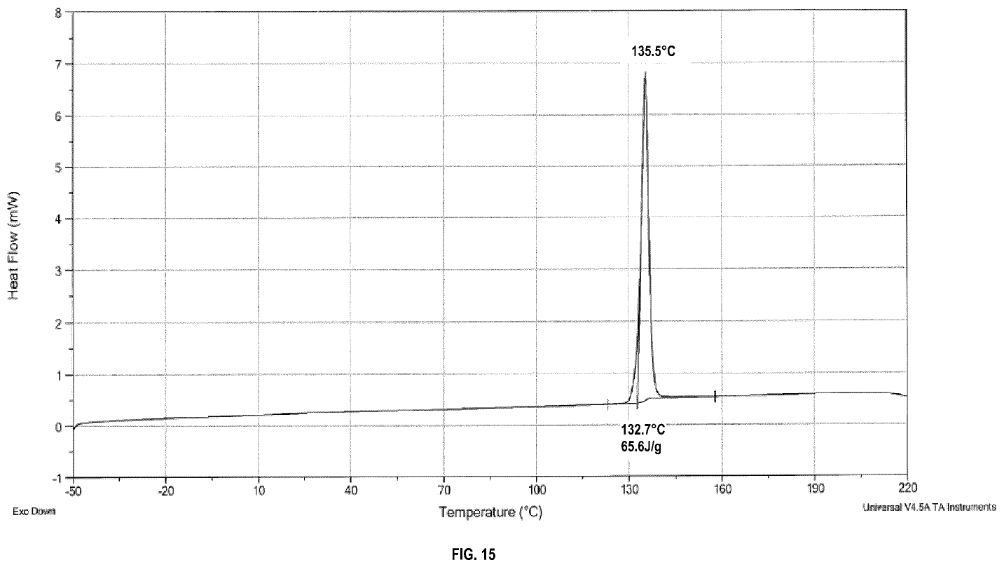

Crystalline Form A may have an exothermic peak which has a starting point at about 132° C. and its highest point at about 136° C. in a differential scanning calorimetry (DSC, 10° C./min). Crystalline Form A may have an exothermic peak at about 135.5±4° C. in a DSC (1000/min). (See FIG. 15.)

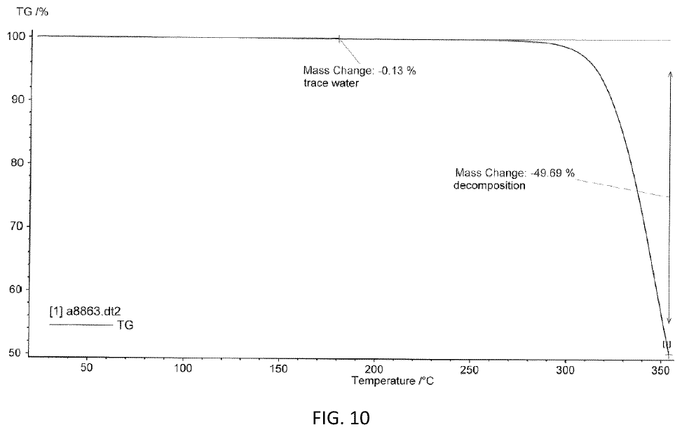

Crystalline Form A may have, in a thermogravimetric analysis (TG-FTIR), a weight loss of about 0.50% or less at the temperature of up to about 180° C., and may decompose at or above 280° C. In an embodiment, at the temperature of up to about 180° C., Crystalline Form A may have a weight loss of about 0.45% or less, about 0.40% or less, about 0.35% or less, about 0.30% or less, about 0.25% or less, about 0.20% or less, or about 0.15% or less. (See FIG. 10.)

In another aspect of the present invention, Crystalline Form A of the compound of Formula 1 may be in a substantially pure form.

In one embodiment, Crystalline Form A of the compound of Formula 1 may have 95% or greater purity in crystalline form.

In one embodiment, Crystalline Form A of the compound of Formula 1 may exhibit peak intensities and d-spacing in A corresponding to the angles set forth below in Table 1:

TABLE 1

| Angle in °2θ | d-spacing in Å | Intensity % |
|---|---|---|
| 3.53 | 25.00 | 62 |
| 7.10 | 12.43 | 29 |
| 8.94 | 9.88 | 22 |
| 9.12 | 9.69 | 28 |
| 9.63 | 9.18 | 20 |
| 10.68 | 8.28 | 18 |
| 11.43 | 7.73 | 13 |
| 12.63 | 7.00 | 15 |
| 13.95 | 6.35 | 15 |
| 16.86 | 5.25 | 14 |
| 18.06 | 4.91 | 74 |
| 18.39 | 4.82 | 31 |
| 18.90 | 4.69 | 37 |
| 19.48 | 4.55 | 43 |
| 20.10 | 4.41 | 27 |
| 21.00 | 4.23 | 32 |
| 21.68 | 4.10 | 12 |
| 22.00 | 4.04 | 41 |
| 22.26 | 3.99 | 100 |
| 22.71 | 3.91 | 32 |
| 23.16 | 3.84 | 13 |
| 23.84 | 3.73 | 22 |
| 24.24 | 3.67 | 20 |
| 24.59 | 3.62 | 14 |
| 25.53 | 3.49 | 13 |
| 26.86 | 3.32 | 8 |
| 27.40 | 3.25 | 11 |
| 27.64 | 3.22 | 10 |
| 28.13 | 3.17 | 9 |
| 28.57 | 3.12 | 17 |
| 28.82 | 3.10 | 15 |
| 29.11 | 3.07 | 8 |
| 29.49 | 3.03 | 9 |
| 29.68 | 3.01 | 9 |
| 29.94 | 2.98 | 9 |
| 30.19 | 2.96 | 8 |
| 30.58 | 2.92 | 9 |
| 31.09 | 2.87 | 9 |
| 31.66 | 2.82 | 7 |
| 32.00 | 2.80 | 6 |
| 32.60 | 2.74 | 8 |
| 32.93 | 2.72 | 8 |
| 33.73 | 2.66 | 8 |
| 34.21 | 2.62 | 6 |
| 35.77 | 2.51 | 7 |
| 36.53 | 2.46 | 23 |
| 36.78 | 2.44 | 10 |
| 37.13 | 2.42 | 10 |
| 37.46 | 2.40 | 7 |
| 37.75 | 2.38 | 7 |
| 38.41 | 2.34 | 8 |
| 39.24 | 2.29 | 5 |
| 39.98 | 2.25 | 10 |
| 40.79 | 2.21 | 7 |

Crystalline Form A of the compound of Formula 1 was found to have unexpectedly and significantly improved (i.e., reduced) hygroscopicity, and Crystalline Form A is more stable than Form B (see Examples 27a and 27b).

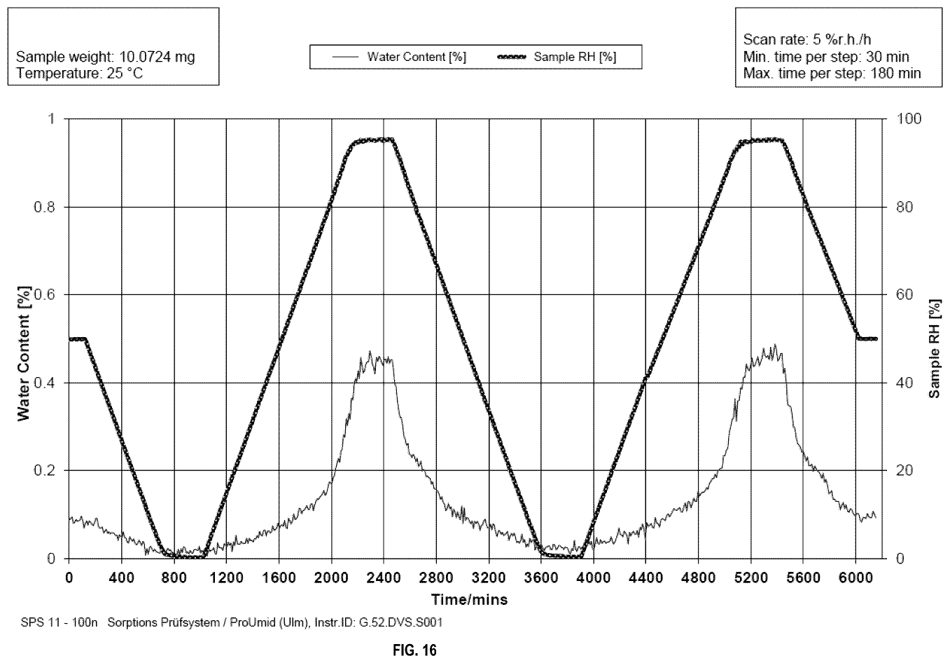

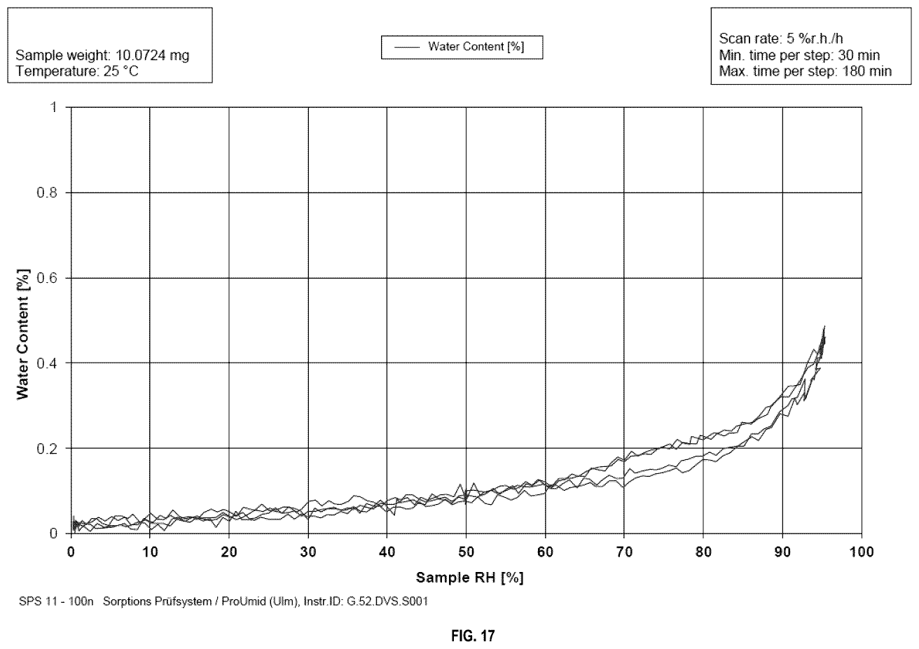

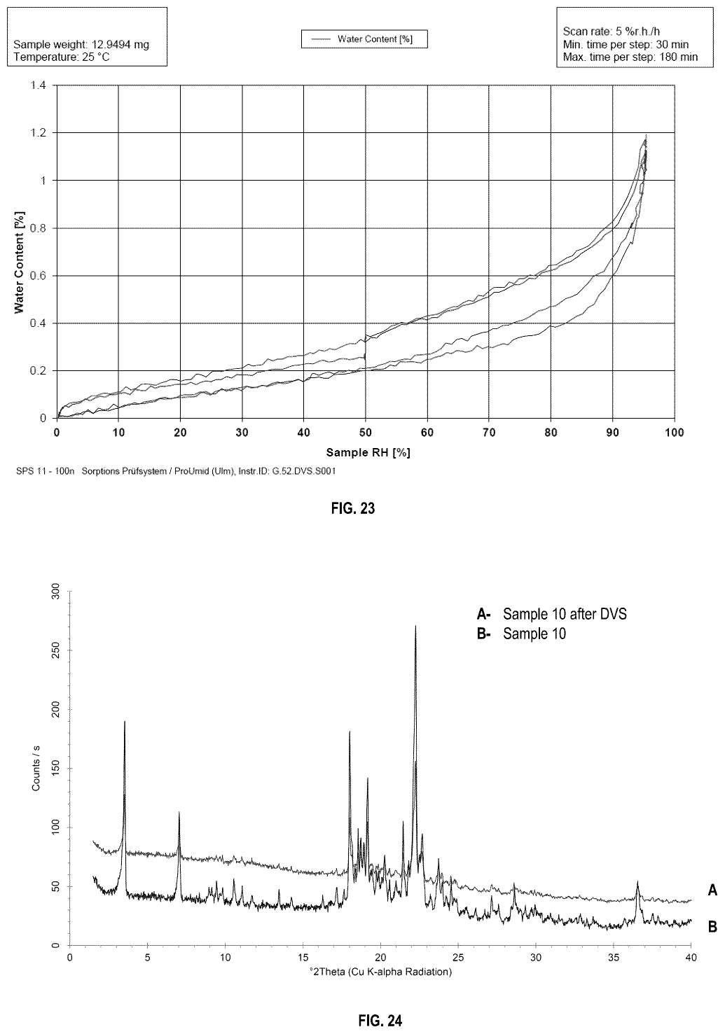

Crystalline Form A of the compound of Formula 1 is an anhydrous form, which contains a trace of residual water. According to hygroscopicity testing (via DVS), Crystalline Form A is slightly hygroscopic, absorbing almost 0.5% of water upon storage at 95% relative humidity for five hours (see FIGS. 16 and 17). In contrast, Crystalline Form B of the compound of Formula 1 absorbed up to 1.2% of water after storage at 95% relative humidity for five hours (see FIGS. 22 and 23). (The amorphous form of the compound of Formula 1 absorbed almost 1.5% of water upon storage at 95% relative humidity for five hours. See FIGS. 4 and 5.)

As shown in the instant disclosure, Crystalline Form A of the compound of Formula 1 exhibited up to 98.7 area-% (determined by HPLC) (see example 24l).

According to results of the competitive slurry experiments (see example 27a), Crystalline Form A of the compound of Formula 1 is more stable than Crystalline Form B of the same compound over the temperature range of 25° C. to 75° C. As the melting temperature and the enthalpy of fusion of Form A are higher than those of Form B, Form A is the stable form (over at least the range 25° C. to 75° C.), and the two forms are monotropically related.

With respect to Crystalline Form C, Crystalline Form A is the stable form at room temperature, and Form C is the stable form at 30° and above (see example 5b).

### Form B

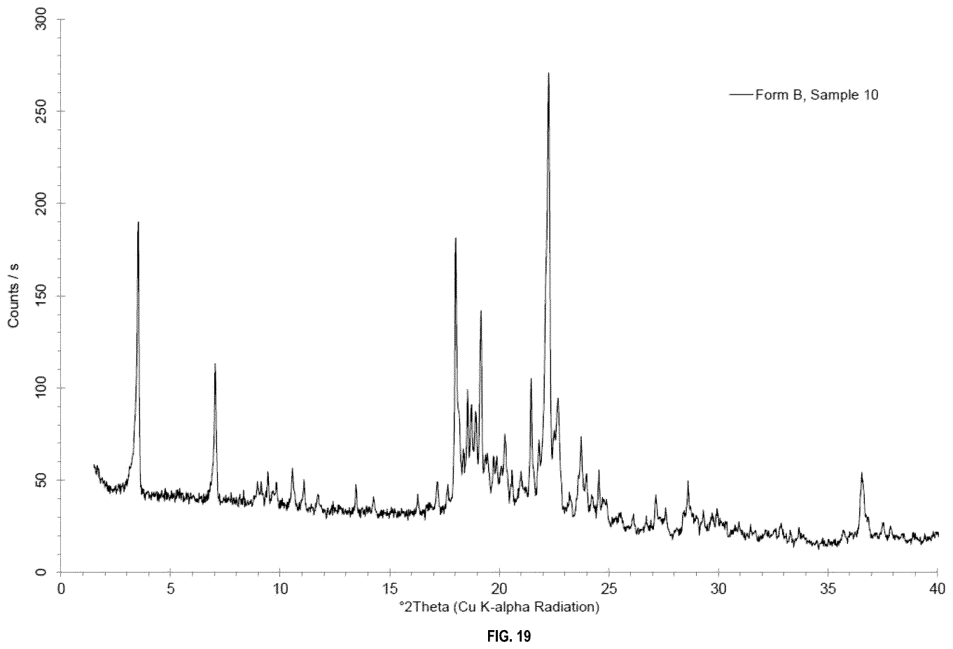

One aspect of the present disclosure provides a crystalline form of the compound of Formula 1, characterized in exhibiting an X-ray powder diffraction (XRPD) pattern comprising peaks at diffraction angles 2θ±0.2° values of 3.5°, 19.2°, and 22.3°. Hereinafter, this crystalline form is referred to as Crystalline Form B. (See FIG. 19.)

In one aspect, Crystalline Form B may exhibit an XRPD pattern comprising peaks at three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, preferably four or more, 2θ±0.2° values selected from the group consisting of 3.5°, 7.1°, 9.1°, 9.4°, 9.8°, 10.6°, 11.1°, 13.5°, 17.7°, 18.0°, 18.6°, 18.9°, 19.2°, 19.5°, 19.8°, 20.3°, 21.0°, 21.5°, 22.3°, and 22.7°.

In another aspect, Crystalline Form B may exhibit an XRPD pattern comprising peaks at three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, preferably four or more, 2θ±0.2° values selected from the group consisting of 3.50°, 7.05°, 9.12°, 9.42°, 9.82°, 10.59°, 11.08°, 13.45°, 17.65°, 18.04°, 18.56°, 18.94°, 19.17°, 19.45°, 19.80°, 20.28°, 20.99°, 21.47°, 22.26°, and 22.69°.

In particular, Crystalline Form B may exhibit an XRPD pattern comprising peaks at 2θ±0.2° values of 3.5°, 7.1°, 9.1°, 9.4°, 9.8°, 11.1°, 13.5°, 17.7°, 18.0°, 19.2°, 19.8°, 21.5°, 22.3°, and 22.7°.

In more particularity, Crystalline Form B may exhibit an XRPD pattern comprising peaks at 2θ±0.2° values of 3.5°, 7.1°, 9.1°, 9.4°, 9.8°, 10.6°, 11.1°, 13.5°, 17.7°, 18.0°, 18.6°, 18.9°, 19.2°, 19.5°, 19.8°, 20.3°, 21.0°, 21.5°, 22.3°, and 22.7°.

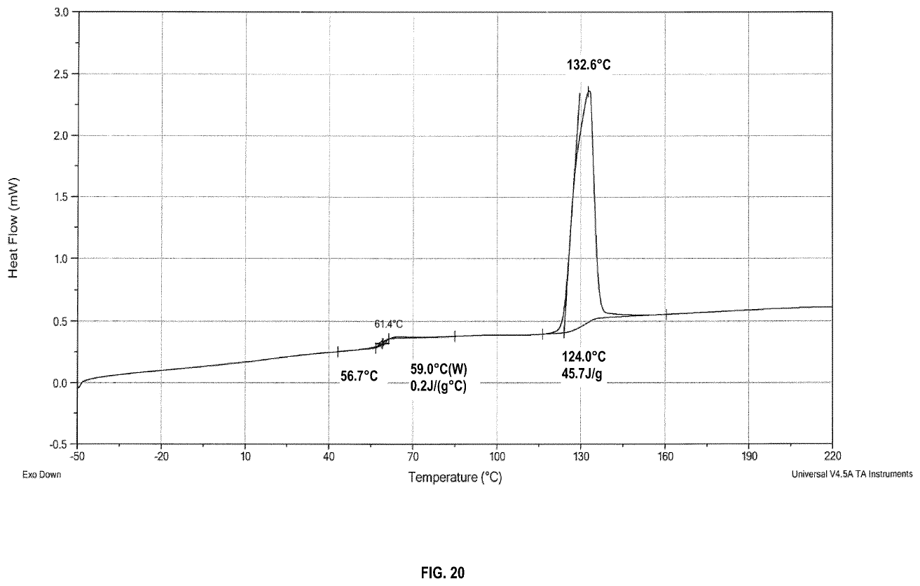

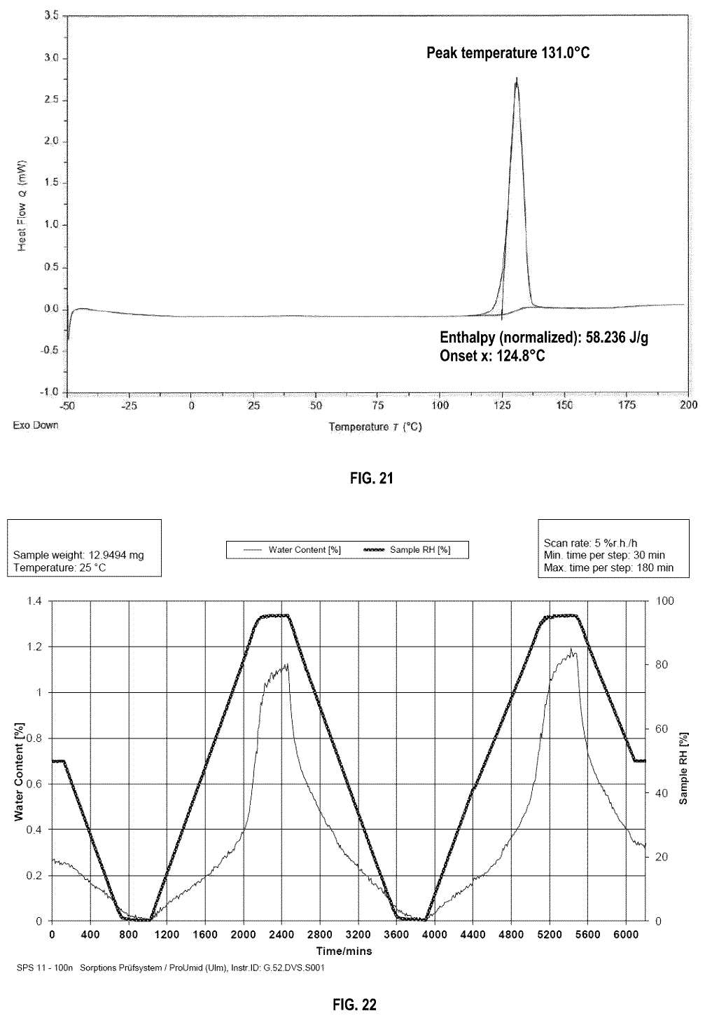

Crystalline Form B may have an exothermic peak which has a starting point at about 124° C. and its highest point at about 133° C. in a differential scanning calorimetry (DSC, 10° C./min). Crystalline Form B may have an exothermic peak at about 132.6±4° C. in a DSC (10° C./min). (See FIGS. 20 and 21.)

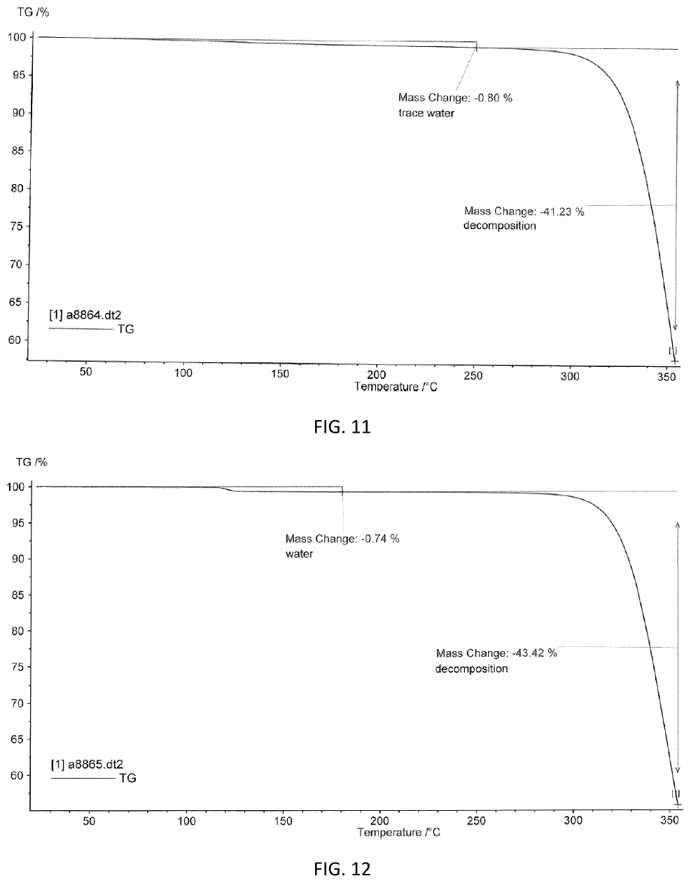

Crystalline Form B may have, in a thermogravimetric analysis (TG-FTIR), a weight loss of about 1.0% or less at the temperature of up to about 250° C., and may decompose at or above 280° C. In an embodiment, at the temperature of up to about 250° C., Crystalline Form B may have a weight loss of about 0.95% or less, about 0.90% or less, about 0.85% or less, or about 0.80% or less. (See FIG. 11.)

In another aspect of the present invention, Crystalline Form B of the compound of Formula 1 may be in a substantially pure form.

In one embodiment, Crystalline Form B of the compound of Formula 1 may have 95% or greater purity in crystalline form.

In one embodiment, Crystalline Form B of the compound of Formula 1 may exhibit peak intensities and d-spacing in A corresponding to the angles set forth below in Table 2:

TABLE 2

| Angle in °2θ | d-spacing in Å | Intensity % |
|---|---|---|
| 3.50 | 25.23 | 78 |
| 7.05 | 12.54 | 47 |
| 9.12 | 9.69 | 23 |
| 9.42 | 9.38 | 23 |
| 9.82 | 9.00 | 23 |
| 10.59 | 8.35 | 25 |
| 11.08 | 7.98 | 24 |
| 11.70 | 7.56 | 20 |
| 13.45 | 6.58 | 21 |
| 14.29 | 6.19 | 19 |
| 16.28 | 5.44 | 19 |
| 17.16 | 5.16 | 21 |
| 17.65 | 5.02 | 21 |
| 18.04 | 4.91 | 67 |
| 18.56 | 4.78 | 36 |
| 18.94 | 4.68 | 34 |
| 19.17 | 4.63 | 55 |
| 19.45 | 4.56 | 27 |
| 19.80 | 4.48 | 25 |
| 20.28 | 4.37 | 30 |
| 20.59 | 4.31 | 21 |
| 20.99 | 4.23 | 23 |
| 21.47 | 4.14 | 39 |
| 21.84 | 4.07 | 28 |
| 22.26 | 3.99 | 100 |
| 22.69 | 3.92 | 39 |
| 23.26 | 3.82 | 17 |
| 23.74 | 3.74 | 27 |
| 23.96 | 3.71 | 23 |
| 24.26 | 3.67 | 17 |
| 24.55 | 3.62 | 22 |
| 24.84 | 3.58 | 16 |
| 25.53 | 3.49 | 14 |
| 26.13 | 3.41 | 13 |
| 26.72 | 3.33 | 12 |
| 27.16 | 3.28 | 16 |
| 27.59 | 3.23 | 14 |
| 28.62 | 3.12 | 18 |
| 29.72 | 3.00 | 13 |
| 29.94 | 2.98 | 14 |
| 30.92 | 2.89 | 11 |
| 32.16 | 2.78 | 10 |
| 32.86 | 2.72 | 11 |
| 33.70 | 2.66 | 9 |
| 35.76 | 2.51 | 9 |
| 36.57 | 2.46 | 19 |
| 37.50 | 2.40 | 10 |
| 37.86 | 2.37 | 10 |
| 38.40 | 2.34 | 9 |
| 38.94 | 2.31 | 9 |
| 40.01 | 2.25 | 9 |

### Form C

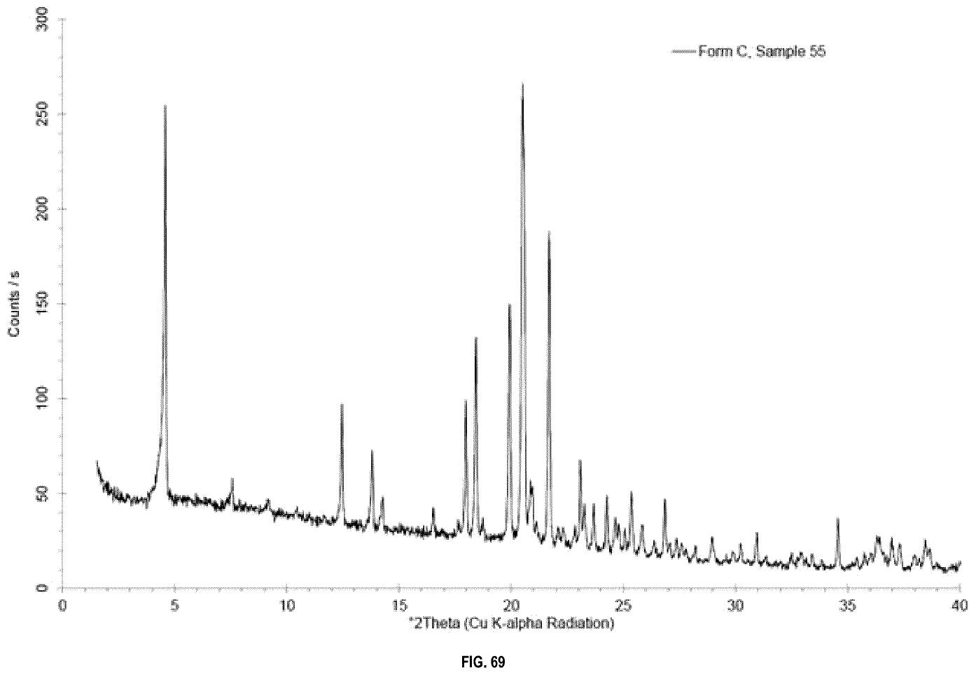

One aspect of the present disclosure provides a crystalline form of the compound of Formula 1, characterized in exhibiting an X-ray powder diffraction (XRPD) pattern comprising peaks at diffraction angles 2θ±0.2° values of 4.6°, 20.5°, and 21.7°. Hereinafter, this crystalline form is referred to as Crystalline Form C. (See FIG. 69.)

In one aspect, Crystalline Form C may exhibit an XRPD pattern comprising peaks at three or more, four or more, five or more, six or more, seven or more, eight or more, preferably four or more, 2θ±0.2° values selected from the group consisting of 4.6°, 7.6°, 12.4°, 13.8°, 18.0°, 18.4°, 20.0°, 20.5°, and 21.7°.

In another aspect, Crystalline Form C may exhibit an XRPD pattern comprising peaks at three or more, four or more, five or more, six or more, seven or more, eight or more, preferably four or more, 2θ±0.2° values selected from the group consisting of 4.58°, 7.56°, 12.44°, 13.79°, 17.98°, 18.42°, 19.92°, 20.54°, and 21.69°.

In particular, Crystalline Form C may exhibit an XRPD pattern comprising peaks at 2θ±0.2° values of 4.6°, 12.4°, 18.0°, 18.4°, 20.0°, 20.5°, and 21.7°.

In more particularity, Crystalline Form C may exhibit an XRPD pattern comprising peaks at 2θ±0.2° values of 4.6°, 7.6°, 12.4°, 13.8°, 18.0°, 18.4°, 20.0°, 20.5°, and 21.7°.

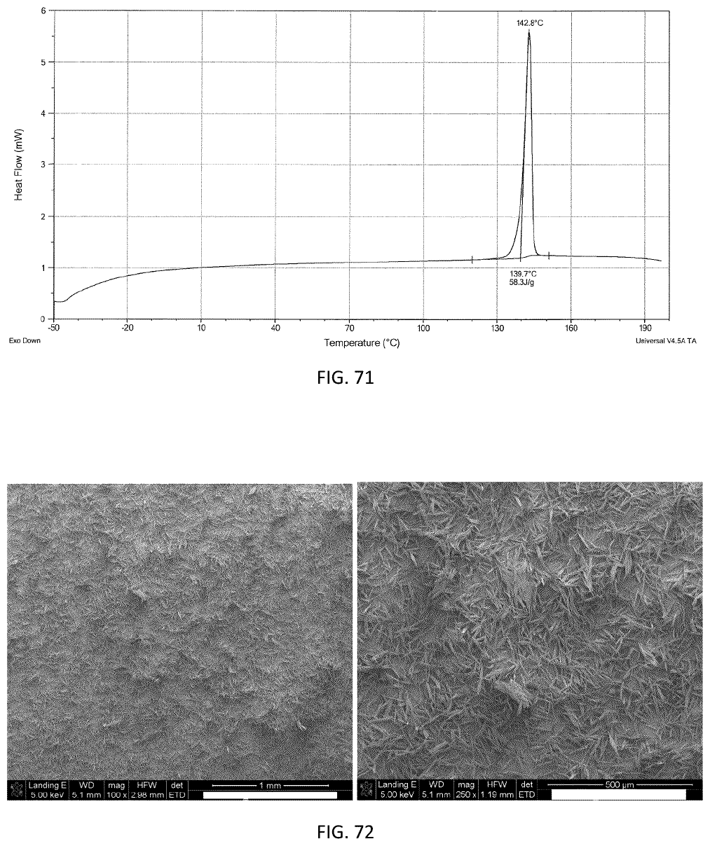

Crystalline Form C may have an exothermic peak which has a starting point at about 140° C. and its highest point at about 143° C. in a differential scanning calorimetry (DSC, 10° C./min). Crystalline Form C may have an exothermic peak at about 142.8±4° C. in a DSC (1000/min). (See FIG. 71.)

In another aspect of the present invention, Crystalline Form C of the compound of Formula 1 may be in a substantially pure form.

In one embodiment, Crystalline Form C of the compound of Formula 1 may have 95% or greater purity in crystalline form.

In one embodiment, Crystalline Form C of the compound of Formula 1 may exhibit peak intensities and d-spacing in A corresponding to the angles set forth below in Table 3:

TABLE 3

| Angle in °2θ | d-spacing in Å | Intensity % |
|---|---|---|
| 4.58 | 19.29 | 96 |
| 7.56 | 11.69 | 22 |
| 9.17 | 9.64 | 18 |
| 12.44 | 7.11 | 37 |
| 13.79 | 6.42 | 27 |
| 14.23 | 6.22 | 18 |
| 16.54 | 5.36 | 16 |
| 17.65 | 5.02 | 14 |
| 17.98 | 4.93 | 37 |
| 18.42 | 4.81 | 50 |
| 18.72 | 4.74 | 14 |
| 19.92 | 4.45 | 56 |
| 20.54 | 4.32 | 100 |
| 20.90 | 4.25 | 21 |
| 21.18 | 4.19 | 12 |
| 21.69 | 4.09 | 71 |
| 22.09 | 4.02 | 12 |
| 22.32 | 3.98 | 12 |
| 22.82 | 3.89 | 11 |
| 23.08 | 3.85 | 26 |
| 23.30 | 3.81 | 17 |
| 23.69 | 3.75 | 17 |
| 24.27 | 3.66 | 18 |
| 24.65 | 3.61 | 14 |
| 25.08 | 3.55 | 12 |
| 25.36 | 3.51 | 19 |
| 25.85 | 3.44 | 13 |
| 26.37 | 3.38 | 10 |
| 26.86 | 3.32 | 18 |
| 27.38 | 3.26 | 10 |
| 28.20 | 3.16 | 8 |
| 28.98 | 3.08 | 10 |
| 29.90 | 2.99 | 7 |
| 30.24 | 2.95 | 9 |
| 30.94 | 2.89 | 11 |
| 31.33 | 2.85 | 6 |
| 32.48 | 2.75 | 6 |
| 32.92 | 2.72 | 7 |
| 33.42 | 2.68 | 7 |
| 33.84 | 2.65 | 6 |
| 34.57 | 2.59 | 14 |
| 35.41 | 2.53 | 6 |
| 35.77 | 2.51 | 7 |
| 36.04 | 2.49 | 7 |
| 36.36 | 2.47 | 10 |
| 36.98 | 2.43 | 10 |
| 37.33 | 2.41 | 9 |
| 37.99 | 2.37 | 7 |
| 38.65 | 2.33 | 8 |
| 39.70 | 2.27 | 5 |
| 40.02 | 2.25 | 5 |
| 40.47 | 2.23 | 7 |
| 41.07 | 2.20 | 6 |
| 41.49 | 2.17 | 6 |
| 42.40 | 2.13 | 8 |
| 42.82 | 2.11 | 8 |
| 43.38 | 2.08 | 5 |
| 43.68 | 2.07 | 5 |
| 45.59 | 1.99 | 5 |
| 46.50 | 1.95 | 6 |
| 47.24 | 1.92 | 6 |

For Crystalline Forms A, B, and C of the compound of Formula 1, the XRPD pattern may be obtained when irradiated with a Cu-Kα light source, for example, using a D8 Advance (Bruker ASX, Germany) analyzer, or when irradiated with a CuKα1 light source, for example, using a STOE STADI P analyzer equipped with a Mythen1K Detector. The Cu-Kα (D8 Advance, Bruker) or Cu-Kα1 (STOE STADI P) light source may have the wavelength of 1.5406 Å.

These peaks may be those having a relative intensity (I/Iₒ) of about 10%, more specifically, about 15% or greater. In an embodiment, these peaks may be those having a relative intensity (I/Iₒ) of about 10% or greater, about 11% or greater, about 12% or greater, about 13% or greater, about 14% or greater, about 15% or greater, about 16% or greater, about 17% or greater, about 18% or greater, about 19% or greater, about 20% or greater, about 21% or greater, about 22% or greater, about 23% or greater, about 24% or greater, about 25% or greater, about 26% or greater, about 27% or greater, about 28% or greater, about 29% or greater, about 30% or greater, about 31% or greater, about 32% or greater, about 33% or greater, about 34% or greater, about 35% or greater, about 36% or greater, about 37% or greater, about 38% or greater, about 37% or greater, or about 40% or greater.

The term “substantially pure” as used herein means at least 95% purity, preferably 97% purity, or more preferably 99% purity, wherein 95% purity means no more than 5%, 97% purity means no more than 3%, and 99% purity means no more than 1%, of any other form of the compound of Formula 1 (other crystalline form, amorphous form, and so forth) being present.

### Method of Preparing Crystalline Form A of the Compound of Formula 1

Another aspect of the present invention provides a method of preparing Crystalline Form A of the compound of Formula 1.

In one embodiment, the method comprises:
dissolving, optionally with stirring and/or heating, a compound of Formula 1,

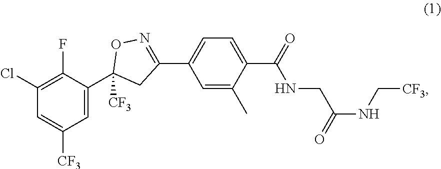 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

in one or more components A, wherein the compound of Formula 1 is an amorphous form, one or more crystalline forms, or a combination thereof, and wherein component A is an organic solvent suitable for dissolving the compound of Formula 1, non-limiting examples of which include C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine;
optionally adding one or more components B, wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane; and filtering resulting solid.

In another embodiment, the method comprises steps of:
dissolving, optionally with stirring and/or heating, a compound of Formula 1,

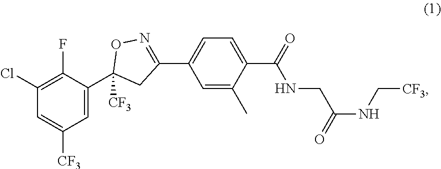 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

in one or more components A, wherein the compound of Formula 1 is an amorphous form, one or more crystalline forms, or a combination thereof, and wherein component A is an organic solvent suitable for dissolving the compound of Formula 1, non-limiting examples of which include C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine;
optionally adding one or more components B, wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane;
optionally evaporating the one or more components A and, if present, the one or more components B; and
filtering, washing, and drying the solid.

In another embodiment, the method comprises steps of:
dissolving, with stirring and heating, a compound of Formula 1,

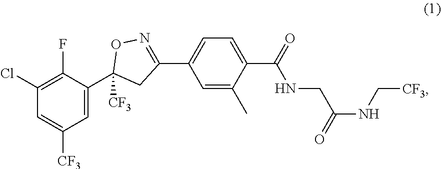 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

in one or more components A, wherein the compound of Formula 1 is an amorphous form, one or more crystalline forms, or a combination thereof, and wherein component A is an organic solvent suitable for dissolving the compound of Formula 1, non-limiting examples of which include C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine;
optionally adding one or more components B, wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane;
optionally evaporating the one or more components A;
seeding a suspension with already-prepared Crystalline Form A of the compound of Formula 1;
optionally adding one or more components B, wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane;
   cooling the mixture;
   temperature cycling the mixture;
   stirring the mixture;
   filtering, washing, and drying the solid.

In an embodiment for large-scale production of Crystalline Form A of the compound of Formula 1,

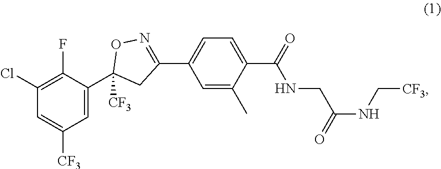 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

the method comprises steps of:
(a) dissolving the compound of Formula 1 in ethyl acetate, optionally 50-100 mL, optionally 70 mL, to form a solution;
(b) heating the solution from (a) to between 50-75° C., optionally 60° C., with about 1 K/minute;
(c) adding between 200-400 mL, optionally 280 mL, of heptane with 2 mL/min. at between 50-75° C., optionally 60° C.;
(d) seeding the resulting fine suspension with already-prepared Crystalline Form A of the compound of Formula 1 (about 0.1 wt. %) at between 50-75° C., optionally 60° C.;
(e) cooling to between 15-25° C., optionally 20° C., at about 2 K/hour;
(f) temperature cycling: wait 1 hour at between 15-25° C., optionally 20° C.; heat to between 35-45° C., optionally 40° C., with about 15 K/hour; then cool to between 15-25° C., optionally 20° C. with about 5 K/hour; repeat 2-5 times, optionally 3 times;
(g) stirring at between 15-25° C., optionally 20° C. for approximately 1 day;
(h) filtering over fritted glass (porosity 4), wash with 30 mL ethyl acetate/heptane (1:4) mixture, optionally 30 mL;
(i) drying the filter cake, optionally for about 2 hours, on the filter, optionally with applied vacuum;
(j) drying the recovered powder overnight, <5 mbar, room temperature.

In yet another embodiment for large-scale production of Crystalline Form A of the compound of Formula 1,

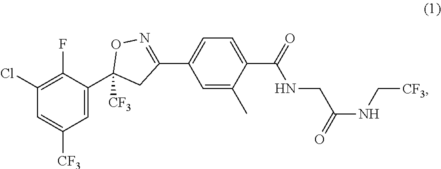 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

the method comprises steps of:
(a) dissolving the compound of Formula 1 in isopropanol, optionally 60-100 mL, optionally 80 mL, to form a solution;
(b) heating the solution from (a) to between 50-75° C., optionally 60° C., with about 1 K/minute;
(c) adding between 100-200 mL, optionally 160 mL, of water with 1 mL/min. at between 50-75° C., optionally 60° C.;
(d) seeding the resulting fine suspension with already-prepared Crystalline Form A of the compound of Formula 1 (about 0.1 wt. %) at between 50-750°, optionally 60° C.;
(e) cooling to between 15525°, optionally 20° C., at about 2 K/hour;
(f) temperature cycling: wait 1 hour at between 15-25° C., optionally 20° C.; heat to between 35-45° C., optionally 40° C., with about 15 K/hour; then cool to between 15-25° C., optionally 2000 with about 5 K/hour; repeat 2-5 times, optionally 3 times;
(g) stirring at between 15-25° C., optionally 20° C. for approximately 1 day;
(h) filtering over fritted glass (porosity 4), wash with 30 mL isopropanol/water (1:2) mixture, optionally 30 mL
(i) drying the filter cake, optionally for about 2 hours, on the filter, optionally with applied vacuum;
(j) drying the recovered powder overnight, <5 mbar, room temperature.

### Method of Preparing Crystalline Form B of the Compound of Formula 1

Another aspect of the present invention provides a method of preparing Crystalline Form B of the compound of Formula 1.

In one embodiment, the method comprises:
dissolving, optionally with stirring and/or heating, a compound of Formula 1,

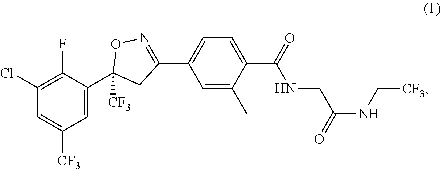 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

in one or more components A, wherein component A is an organic solvent suitable for dissolving the compound of Formula 1, non-limiting examples of which include C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine; and
optionally adding one or more components B, wherein component B is an antisolvent that reduces the solubility of the mixture, wherein non-limiting examples of component B include one or more of water and C₅-C₁₂ cyclic or acyclic hydrocarbon alkanes (e.g., cyclohexane, hexane, heptane, n-heptane, octane, nonane, and decane), preferably one or more of water and heptane;
evaporating the one or more components A and, if present, the one or more components B; and
filtering resulting suspension.

In another embodiment, the method comprises steps of:
dissolving, optionally with stirring and/or heating, a compound of Formula

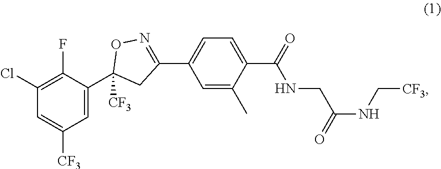 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

In component A, wherein the compound of Formula 1 is an amorphous form, one or more crystalline forms, or a combination thereof, and wherein component A is an organic solvent suitable for dissolving the compound of Formula 1, non-limiting examples of which include one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine; and
adding one or more components B, wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane;
evaporating the one or more components A and the one or more components B; and filtering resulting suspension.

### Method of Preparing Crystalline Form C of the Compound of Formula 1

Another aspect of the present invention provides a method of preparing Crystalline Form C of the compound of Formula 1.

In one embodiment, the method comprises:
dissolving, optionally with stirring and/or heating, a compound of Formula 1,

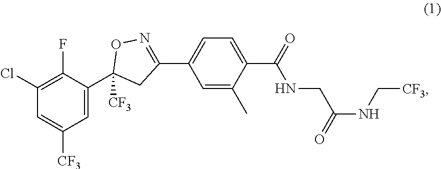 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

in one or more components A, wherein the compound of Formula 1 dissolved in the dissolving step is an amorphous form, one or more crystalline forms, or a combination thereof, and wherein component A is an organic solvent suitable for dissolving the compound of Formula 1, non-limiting examples of which include C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, ethyl lactate, isopropyl acetate, isobutyl acetate, methyl ethyl ketone (MEK), methanol, medium-chain triglycerides (e.g., MIGLYOL® 812), N-methyl-2-pyrrolidone (NMP), 1-octanol, 1-propanol, 2-propanol, methyl tert-butyl ether (TBME), tetrahydrofuran (THF), toluene, and triethylamine; and
adding one or more components B, wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane;
filtering the resulting solid.

In another embodiment, the method comprises:
dissolving, optionally with stirring, a compound of Formula 1,

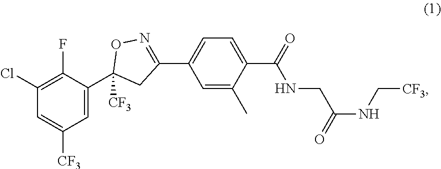 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

in one or more components A at a temperature of between about 50-70° C., optionally about 60° C., wherein the compound of Formula 1 dissolved in the dissolving step is an amorphous form, one or more crystalline forms, or a combination thereof, and wherein component A is an organic solvent suitable for dissolving the compound of Formula 1, non-limiting examples of which include C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; preferred non-limiting examples of component A include one or more of C₁-C₄ alcohol, acetone, acetonitrile, aniline, anisole, benzyl alcohol, butyronitrile, chloroform, DMSO, ethanol, ethyl acetate, ethyl lactate, isopropyl acetate, isobutyl acetate, methyl ethyl ketone (MEK), methanol, medium-chain triglycerides (e.g., MIGLYOL® 812), N-methyl-2-pyrrolidone (NMP), 1-octanol, 1-propanol, 2-propanol, methyl tert-butyl ether (TBME), tetrahydrofuran (THF), toluene, and triethylamine; and
optionally adding one or more components B, wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably one or more of water and heptane.
cooling the solution formed from the dissolving and optional adding step to between about 35-60° C., optionally about 50° C.;
optionally concentrating, optionally evaporating, the cooled solution;
filtering; and
drying.

In another embodiment, the method comprises steps of:
(a) dissolving, optionally with stirring, t the compound of Formula 1 in a mixture of one or more components A and one or more components B at a temperature of between about 50-70° C., optionally about 60° C., wherein the compound of Formula 1 dissolved in the dissolving step is an amorphous form, one or more crystalline forms, or a combination thereof,
wherein component A is an organic solvent suitable for dissolving amorphous form of the compound of Formula 1, non-limiting examples of which include C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine, optionally ethyl acetate;
wherein component B is an antisolvent that reduces the solubility of the mixture, wherein non-limiting examples of component B include water and C₅-C₁₂ cyclic or acyclic hydrocarbon alkanes (e.g., cyclohexane, hexane, heptane, n-heptane, octane, nonane, and decane), preferably heptane;
(b) stirring until a suspension forms;
(c) cooling the solution formed from dissolving the compound of Formula 1 in component A and in component B to between about 35-60° C., optionally about 45° C.;
(d) evaporating the one or more components A and the one or more components B optionally under nitrogen flow;
(e) adding additional component B, optionally heptane, and stirring;
(f) evaporating the one or more components A and the one or more components B, optionally with nitrogen flow, optionally with weak nitrogen flow, at a temperature between about 35-60° C., optionally about 45° C.;
(g) filtering the suspension over fritted glass (porosity);
(h) drying the filter, optionally with applied vacuum.

In an embodiment, if component B is added in any of the above methods or any of the methods of the instant disclosure, the ratio by volume (v/v) of component A to component B may be optionally be in a range of about 20:1 to about 1:20. In an embodiment, the ratio by volume (v/v) of component A to component B is in a range of about 10:1 to about 1:10, about 8:1 to about 1:8, about 5:1 to about 1:5, about 3:1 to about 1:3, about 2:1 to about 1:2. In an embodiment, the ratio by volume (v/v) of component A to component B is about 3:1, about 2:1, about 1:1, about 4:5, about 1:3, about 1:10.

In the dissolving step of the any of the methods in the instant disclosure, compound of Formula 1, which may be amorphous and/or crystalline, may be dissolved in one or more solvents selected from a group consisting of a C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, isobutyl acetate, MEK, methanol, medium-chain triglycerides, NMP, 1-octanol, 1-propanol, 2-propanol, TBME, THF, and triethylamine. The solvent may be, for example, a single solvent, such as methanol, ethanol, isopropyl alcohol, acetone, ethyl acetate, isopropyl acetate, and methyl t-butyl ether, or a mixed solvent thereof, e.g., a mixed solvent of methanol and methyl t-butyl ether.

The compound of Formula 1 may be prepared according to the methods disclosed in US'365 and WO'188, the disclosures of which are incorporated herein in its entirety by reference. However, the embodiments described herein are not limited thereto, and the amorphous form of the compound of Formula 1 may be prepared using any method known to the person of ordinary skill in the relevant art.

The washing and drying steps are not specifically limited. The washing may be performed using the solvent used in the dissolving step. The drying may be performed using any method which does not affect the stability of the crystalline form of the compound of Formula 1, for example, at a temperature of about 40° C. to about 50° C. for about 15 hours to 30 hours.

In the case where solvents other than those described above are used for crystallization, another crystalline form may be obtained.

### Medical Usage and Pharmaceutical Composition

Another aspect of the present invention provides a pharmaceutical composition comprising one or more crystalline forms of the compound of Formula 1 as active ingredient(s) and at least one pharmaceutically acceptable carrier or excipient. In another aspect, the one or more crystalline forms of the compound of Formula 1 are selected from the group consisting of Crystalline Forms A, B, and C of the compound of Formula 1.

As disclosed in US'365 and WO'188, the compound of Formula 1 was found to exhibit an EC₅₀ of <1 ppm in an in vitro evaluation of ingesting activity against fleas (Ctenocephalides felis). The same references also disclose that the compound of Formula 1 had a half-life of 50 days.

One or more crystalline forms of the compound of Formula 1, or a pharmaceutical composition thereof, may be used for the treatment and/or control of pests. The term “pests” includes ectoparasites and endoparasites on and in animals and in the hygiene field. Particular pests are fleas, ticks, mites, flies, worms, and lice. Even more particular pests are fleas, flies, mites, and ticks.

Animals in the context of the invention are understood to include vertebrates. The term vertebrate in this context is understood to comprise, for example fishes, amphibians, reptiles, birds, and mammals including humans. One preferred group of vertebrates according to the invention comprises warm-blooded animals including farm animals, such as cattle, horses, pigs, sheep and goats, poultry such as chickens, turkeys, guinea fowls and geese, fur-bearing animals such as mink, foxes, chinchillas, rabbits and the like, as well as companion animals such as ferrets, guinea pigs, rats, hamster, cats and dogs, and also humans. A further group of preferred vertebrates according to the invention comprises fishes including salmons. Particularly preferred animals are cats and dogs.

In the context of the present invention, ectoparasites are understood to be in particular insects, acari (mites and ticks), and crustaceans (sea lice). These include insects of the following orders: Lepidoptera, Coleoptera, Homoptera, Hemiptera, Heteroptera, Diptera, Dictyoptera, Thysanoptera, Orthoptera, Anoplura, Siphonaptera, Mallophaga, Thysanura, Isoptera, Psocoptera and Hymenoptera. However, the ectoparasites which may be mentioned in particular are those which trouble humans or animals and carry pathogens, for example flies such as Musca domestica, Musca vetustissima, Musca autumnalis, Fannia canicularis, Sarcophaga carnaria, Lucilia cuprina, Lucilia sericata, Hypoderma bovis, Hypoderma lineatum, Chrysomyia chloropyga, Dermatobia hominis, Cochliomyia hominivorax, Gasterophilus intestinalis, Oestrus ovis, biting flies such as Haematobia irritans, Haematobia irritans exigua, Stomoxys calcitrans, horse-flies (Tabanids) with the subfamilies of Tabanidae such as Haematopota spp. (e.g. Haematopota pluvialis) and Tabanus spp, (e.g. Tabanus nigrovittatus) and Chrysopsinae such as Chrysops spp. (e.g. Chrysops caecutiens); Hippoboscids such as Melophagus ovinus (sheep ked); tsetse flies, such as Glossinia spp.; other biting insects like midges, such as Ceratopogonidae (biting midges), Simuliidae (Blackflies), Psychodidae (Sandflies); but also blood-sucking insects, for example mosquitoes, such as Anopheles spp, Aedes spp and Culex spp, fleas, such as Ctenocephalides felis and Ctenocephalides canis (cat and dog fleas), Xenopsylla cheopis, Pulex irritans, Ceratophyllus gallinae, Dermatophilus penetrans, blood-sucking lice (Anoplura) such as Linognathus spp, Haematopinus spp, Solenopotes spp, Pediculus humanis; but also chewing lice (Mallophaga) such as Bovicola (Damalinia) ovis, Bovicola (Damalinia) bovis and other Bovicola spp. Ectoparasites also include members of the order Acarina, such as mites (e.g. Chorioptes bovis, Cheyletiella spp., Dermanyssus gallinae, Ortnithonyssus spp., Demodex canis, Sarcoptes scabiei, Psoroptes ovis and Psorergates spp. and ticks. Representatives ticks are, for example, Boophilus, Amblyomma, Anocentor, Dermacentor, Haemaphysalis, Hyalomma, Ixodes, Rhipicentor, Margaropus, Rhipicephalus, Argas, Otobius and Ornithodoros and the like, which preferably infest vertebrates, for example warm-blooded animals including farm animals, such as cattle, horses, pigs, sheep and goats, poultry such as chickens, turkeys, guinea fowls, and geese, fur-bearing animals such as mink, foxes, chinchillas, rabbits and the like, as well as companion animals such as ferrets, guinea pigs, rats, hamster, cats and dogs, but also humans and fishes.

Crystalline forms of the compound of Formula 1 are also active against all or individual development stages of animal pests showing normal sensitivity, as well as those showing resistance to widely used parasiticides. This is especially true for resistant insects and members of the order Acarina. The insecticidal, ovicidal and/or acaricidal effect of the active substances of the invention can manifest itself directly, i.e. killing the pests either immediately or after some time has elapsed, for example when moulting occurs, or by destroying their eggs, or indirectly, e.g. reducing the number of eggs laid and/or the hatching rate, good efficacy corresponding to a pesticidal rate (mortality) of at least 50 to 60%.

Crystalline forms of the compound of Formula 1 can also be used against hygiene pests, especially of the order Diptera of the families Muscidae, Sarcophagidae, Anophilidae and Culicidae; the orders Orthoptera, Dictyoptera (e.g. the family Blattidae (cockroaches), such as Blatella germanica, Blatta orientalis, Periplaneta americana) and Hymenoptera (e.g. the families Formicidae (ants) and Vespidae (wasps).

Crystalline forms of the compounds of formula (I) are also effective against ectoparasites of fishes, especially the sub-class of Copepoda (e.g. order of Siphonostomatoida (sea lice), whilst being well tolerated by fish.

Crystalline forms of the compound of Formula 1 can also be used against worms of the class Cestoda, including the subclasses Eucestoda and Cestodaria.

Crystalline forms of the compound of Formula 1 also have sustainable efficacy on parasitic mites and insects of plants. In the case of spider mites of the order Acarina, they are effective against eggs, nymphs and adults of Tetranychidae (Tetranychus spp. and Panonychus spp.).

Crystalline forms of the compound of Formula 1 have high activity against sucking insects of the order Homoptera, especially against pests of the families Aphididae, Delphacidae, Cicadellidae, Psyllidae, Loccidae, Diaspididae and Eriophydidae (e.g. rust mite on citrus fruits); the orders Hemiptera, Heteroptera and Thysanoptera, and on the plant-eating insects of the orders Lepidoptera, Coleoptera, Diptera and Orthoptera

Crystalline forms of the compound of Formula 1 are similarly suitable as a soil insecticide against pests in the soil.

Crystalline forms of the compound of Formula 1 are therefore effective against all stages of development of sucking insects and eating insects on crops such as cereals, cotton, rice, maize, soya, potatoes, vegetables, fruit, tobacco, hops, citrus, avocados and other crops.

Crystalline forms of the compound of Formula 1 are also effective against plant nematodes of the species Meloidogyne, Heterodera, Pratylenchus, Ditylenchus, Radopholus, Rizoglyphus etc.

Crystalline forms of the compound of Formula 1 are effective against helminths. Helminths are commercially important because they cause serious diseases in mammals and poultry, e.g. in sheep, pigs, goats, cattle, horses, donkeys, camels, dogs, cats, rabbits, guinea-pigs, hamsters, chicken, turkeys, guinea fowls and other farmed birds, as well as exotic birds. Typical nematodes are: Haemonchus, Trichostrongylus, Ostertagia, Nematodirus, Cooperia, Ascaris, Bunostonum, Oesophagostonum, Charbertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. The trematodes include, in particular, the family of Fasciolideae, especially Fasciola hepatica.

The pesticidal activity of crystalline forms of the compound of Formula 1 according to the invention corresponds to a mortality rate of about 50-60% of the pests mentioned, more preferably to a mortality rate over 90%, most preferably to 95-100%. Crystalline forms of the compounds of formula (I) are preferably employed internally and externally in unmodified form or preferably together with the adjuvants conventionally used in the art of formulation and may therefore be processed in a known manner to give, for example, liquid formulations (e.g. spot-on, pour-on, spray-on, emulsions, suspensions, solutions, emulsifiable concentrates, solution concentrates), semi-solid formulations (e.g. creams, ointments, pastes, gels, liposomal preparations) and solid preparations (e.g. food additives tablets including e.g. capsules, powders including soluble powders, granules, or embeddings of the active ingredient in polymeric substances, like implants and microparticles). As with the compositions, the methods of application are selected in accordance with the intended objectives and the prevailing circumstances.

Crystalline forms of the compound of Formula 1 can be administered alone or in the form of a composition. In practice, the compounds of the invention are usually administered in the form of compositions, that is, in admixture with at least one acceptable excipient. The proportion and nature of any acceptable excipient(s) are determined by the properties of the selected compound of the invention, the chosen route of administration, and standard practice as in the veterinary and pharmaceutical fields.

In one embodiment, the present invention provides compositions comprising: one or more crystalline forms of the compound of Formula 1 and at least one acceptable excipient.

In effecting such treatment and/or control, a crystalline form of the compound of Formula 1 can be administered in any form and route which makes the compound bioavailable. Crystalline forms of the compound of Formula 1 can be administered by a variety of routes, including orally, in particularly by tablets and capsules. Crystalline forms of the compound of Formula 1 can be administered parenteral routes, more particularly by inhalation, subcutaneously, intramuscularly, intravenously, intraarterially, transdermally, intranasally, rectally, vaginally, ocularly, topically, sublingually, and buccally, intraperitoneally, intraadiposally, intrathecally and via local delivery for example by catheter or stent.

One skilled in the art can readily select the proper form and route of administration depending upon the particular characteristics of the crystalline form(s) selected, the disorder or condition to be treated, the stage of the disorder or condition, and other relevant circumstances. The pharmaceutical compositions of the invention may be administered to the subject, for example, in the form of tablets, including chewable tablets, capsules, cachets, papers, lozenges, wafers, elixirs, boli, ointments, transdermal patches, aerosols, inhalants, suppositories, drenches, solutions, injections, and suspensions.

The term “acceptable excipient” refers to those excipients typically used in preparing veterinary and pharmaceutical compositions and should be pure and non-toxic in the amounts used. They generally are a solid, semi-solid, or liquid material which in the aggregate can serve as a vehicle or medium for the active ingredient. Some examples of acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

In one embodiment, the composition is adapted for oral administration, such as a tablet or a capsule or a liquid formulation, for example, a solution or suspension, adapted for oral administration. In one embodiment, the composition is adapted for oral administration, such as chewable formulation, adapted for oral administration. In still another embodiment, the composition is a liquid or semi-solid formulation, for example, a solution or suspension or a paste, adapted for parenteral administration.

In one embodiment, the composition is adapted for injection administration, such as a solution or suspension, adapted for injection administration.

Particular compositions for usage on subjects in the treatment and/or control of pests, preferably ectoparasites, comprise solutions; injectables; emulsions including classical emulsions, microemulsions and self-emulsifying compositions, that are waterless organic, preferably oily, compositions which form emulsions, together with body fluids, upon addition to the subject's body; suspensions (drenches); pour-on formulations; food additives; powders; tablets including effervescent tablets; boli; capsules including micro-capsules; and chewable treats. Particularly preferred composition forms are tablets, capsules, food additives or chewable treats.

The compositions of the present invention are prepared in a manner well known in the veterinary and pharmaceutical art and include at least one crystalline form of the compound of Formula 1 as the active ingredient. The amount of crystalline form(s) of the compound 1 of Formula 1 may be varied depending upon its particular form and may conveniently be between 1% to about 50%, preferably about 10% to about 35%, more preferably, about 15% to about 25%, of the weight of the unit dose form. The present pharmaceutical compositions are preferably formulated in a unit dose form, each dose typically containing from about 0.5 mg to about 100 mg of crystalline form(s) of the invention. One or more unit dose form(s) may be taken to affect the treatment dosage. The remainder may be in any other form of the compound of Formula 1 (other possible crystalline forms, amorphous form, and so forth).

In one embodiment, the present invention also provides a method for treating pests, comprising: administering to a subject in need thereof an effective amount of a crystalline form of the compound of Formula 1, the method optionally further comprising an effective amount of at least one additional active compound or co-crystal.

In one embodiment, the present invention also provides a method for controlling pests, comprising: administering to a subject in need thereof an effective amount of a crystalline form of the compound of Formula 1, the method optionally further comprising an effective amount of at least one additional active compound or co-crystal.

In one embodiment, the present invention also provides a method for treating or controlling pests, comprising: contacting a subject's environment with an effective amount of a crystalline form of the compound of Formula 1, the method optionally further comprising an effective amount of at least one additional active compound or co-crystal.

Thus, the invention provides for the use of a crystalline form of the invention as a medicament, including for the manufacture of a medicament. In one embodiment, the invention provides the manufacture of a medicament comprising a crystalline forms of the compound of Formula 1 for treating pests. In one embodiment, the invention provides the manufacture of a medicament comprising a crystalline form of the compound of Formula 1 for controlling pests.

The terms “treating”, “to treat”, “treated”, or “treatment”, include without limitation restraining, slowing, stopping, reducing, ameliorating, reversing the progression or severity of an existing symptom, or preventing a disorder, condition, or disease. For example, an adult heartworm infection would be treated by administering a compound of the invention. A treatment may be applied or administered therapeutically.

The terms “control”, “controlling” or “controlled” refers to include without limitation decreasing, reducing, or ameliorating the risk of a symptom, disorder, condition, or disease, and protecting an animal from a symptom, disorder, condition, or disease. Controlling may refer to therapeutic, prophylactic, or preventative administration. For example, a larvae or immature pest may be asymptomatic but would be controlled by acting on the larvae or immature pest preventing the infection from progressing to a symptomatic or debilitating infection by mature pest.

Thus, the use of the crystalline forms of the invention in the treatment and/or control of pests, in particular ectoparasites, refers to the use of the crystalline forms of the invention to act on the various forms of the pest throughout its life cycle, independent of whether a subject is manifesting a symptom, including morbidity or mortality, and independently of the phase(s) of the challenge.

As used herein, “administering to a subject” includes but is not limited to cutaneous, subcutaneous, intramuscular, mucosal, submucosal, transdermal, oral or intranasal administration. Administration could include injection or topical administration, for example, pour-on or spot-on administration. The pour-on or spot-on method is especially advantageous for use on herd animals such as cattle, horses, sheep or pigs, in which it is difficult or time-consuming to treat all the animals orally or by injection. Because of its simplicity, this method can of course also be used for all other animals, including individual domestic animals or pets, and is greatly favoured by the keepers of the animals, as it can often be carried out without the specialist presence of the veterinarian.

The terms “subject” and “patient” refers includes humans and non-human mammalian animals and fish, the vertebrates described herein, such as dogs, cats, mice, rats, guinea pigs, rabbits, ferrets, cows, horses, sheep, goats, and pigs. Particular subjects are mammalian pets or companion animals, such as dogs and cats and also mice, guinea pigs, ferrets, and rabbits.

The term “effective amount” refers to an amount which gives the desired benefit to the subject and includes administration for both treatment and control. The amount will vary from one individual subject to another and will depend upon a number of factors, including the overall physical condition of the subject and the severity of the underlying cause of the condition to be treated, concomitant treatments, and the amount of a crystalline form of the compound of Formula 1 used to maintain desired response at a beneficial level.

An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, infection, or disease involved; the degree of or involvement or the severity of the condition, disorder, or disease, the response of the individual patient; the particular crystal form administered; the mode of administration; the bioavailability characteristics of the preparation administered; the dose regimen selected; the use of concomitant medication; and other relevant circumstances. An effective amount of the present invention, the treatment dosage, is expected to range from 0.5 mg to 100 mg. Specific amounts can be determined by the skilled person. Although these dosages are based on a subject having a mass of about 1 kg to about 20 kg, the diagnostician will be able to determine the appropriate dose for a subject whose mass falls outside of this weight range. An effective amount of the present invention, the treatment dosage, is expected to range from 0.1 mg to 10 mg/kg of the subject. The dosing regimen is expected to be monthly, quarterly, semi-annual, or annual administration.

The crystalline forms of the compound of Formula 1 may be combined with one or more other active compounds, co-crystals, or therapies for the treatment of one or more disorders, diseases or conditions, including the treatment of pests, for which it is indicated. The crystalline forms of the compound of Formula 1 may be administered simultaneously, sequentially or separately in combination with one or more compounds, co-crystals, or therapies for treating pests and other disorders.

Thus, it is understood that the compositions and methods of the present invention optionally include comprising an effective amount of at least one additional active compound and/or co-crystal. Additional active compounds useful in the present invention include those used to treat fleas, ticks, flies, and mosquitos and include macrocyclic lactones, like milbemycin oxime, imidacloprid, spinosad, pyriproxyfen, premethrin, S-methoprene, praziquantel and moxidectin. Further exemplary addition active compounds include, but are not limited to, afoxolaner, broflanilide, fluralaner, fluxametamide, isocycloseram, lotilaner, modoflaner, nicofluprole, sarolaner, tigolaner, albendazole, cambendazole, fenbendazole, flubendazole, mebendazole, oxfendazole, parabendazole, tiabendazole, triclabendazole, amitraz, demiditraz, clorsulon, closantel, oxyclonazide, rafoxanide, cyphenothrin, deltamethrin, flumethrin, permethrin, cyromazine, derquantel, diamphenetide, dicyclanil, dinotefuran, imidacloprid, nitenpyram, thiamethoxam, abamectin, doramectin, emamectin, eprinomectin, ivermectin, moxidectin, selamectin, milbemycin oxime, emodepside, epsiprantel, fipronil, fluazuron, fluhexafon, indoxacarb, levamisol, lufenuron, metaflumizone, methoprene, monepantel, morantel, niclosamide, nitroscanate, nitroxynil, novaluron, oxantel, praziquantel, pyrantel, pyriprole, pyriproxyfen, sisapronil, spinosad, spinetoram and triflumezopyrim, or a salt of any of the foregoing.

The activity of the compounds of the invention may be determined by a variety of methods, including in vitro and in vivo methods.

When administered to a patient, a total daily dosage of the crystalline form of the compound of Formula 1 may vary depending on the administration route, administration time, types of other compounds used in combination, or the patient's age, sex, weight, status, medical history, and so forth. Therefore, the dosage of the compound may be determined within the range in which a desired therapeutic effect is achieved without causing harmful or serious adverse effects.

The pharmaceutical composition may be in oral or parenteral dosage form.

For oral dosage forms, the carrier used may include sweetening agents, binders, resolvents, solubilizing agents, wetting agents, emulsifying agents, isotonic agents, adsorbents, disintegrating agents, antioxidants, antiseptics, lubricants, fillers, flavoring agents, coating agents, and so forth. For example, the carrier may include lactose, calcium hydrogen phosphate, hydroxypropyl cellulose, carboxymethyl cellulose, colloidal silicon dioxide, fumed silica, magnesium stearate, talc, agar, water, ethanol, polyethylene glycol, polyvinylpyrrolidone, sodium chloride, calcium chloride, orange essence, strawberry essence, vanilla flavor, Opadry white, and so forth.

Examples of available injectable carriers include distilled water, saline, glucose solutions, pseudo-glucose solutions, alcohols, glycol ethers (for example, polyethylene glycol 400), oils, fatty acids, fatty acid esters, glycerides, surfactants, suspending agents, emulsifying agents, and so forth.

The above descriptions of the pharmaceutical composition according to an aspect of the present invention may be applied per se to details of the method for treating and/or controlling pests.

The dose used in the method for treating and/or controlling pests may be an amount effective for the treatment and/or control. The above description of the dose of the pharmaceutical composition may apply per se to the method for treating and/or controlling pests.

The description regarding medical usage and pharmaceutical compositions of the compound of Formula 1 disclosed in US'365 and WO'188 may apply to those of crystalline forms of the compound of Formula 1 according to the present invention.

In an aspect, in any of the above embodiments, the crystalline form(s) of the compound of Formula 1 may be:
(a) selected from the group consisting of Crystalline Forms A, B, and C of the compound of Formula 1;
(b) Crystalline Form A of the compound of Formula 1;
(c) Crystalline Form B of the compound of Formula 1;
(d) Crystalline Form C of the compound of Formula 1;
(e) Crystalline Forms A and B of the compound of Formula 1;
(f) Crystalline Forms A and C of the compound of Formula 1;
(g) Crystalline Forms B and C of the compound of Formula 1;
(h Crystalline Forms A, B, and C of the compound of Formula 1.

Hereinafter, the present invention will be described in more detail with reference to the following Examples. However, these Examples are for illustrative purposes only, and the invention is not intended to be limited by these Examples.

### Analysis Apparatus and Method of Measurement

### 1. X-Ray Powder Diffraction (XRPD)

X-ray powder diffraction (XRPD) analyses of samples were performed in the range from 1.500° 2θ to 50.480° 2θ using a STOE Stadi P Diffraktometer with MYTHEN1K analyzer or a D8 Advance (Bruker ASX, Germany) analyzer. For STOE Stadi P with Mythen1K Detector, samples (about 10 mg to about 20 mg of powder) were measured between two acetate foils or Kapton foils. For D8 Advance analyzer, samples were measured in 0.5-mm deep silicon single-crystal sample holders.

No special treatment was used in preparing the samples other than the application of slight pressure to distribute the powder over the irradiated surface area. An ambient air atmosphere was used for all measurements, and each sample was rotated during the measurement.

The measurement was performed as follows:
Anode material (Kα): Cu-Kα (1.540598 Å), D8 Advance analyzer; or Cu-Kα1 (1.540598 Å), STOE Stadi P Diffraktometer with MYTHEN1K analyzer
Scan range: 1.5° to 50.5°
Generator settings: 40 mA, 40.0 kV
Scan speed: 12 sec/step
Temperature: Room temperature
Step size: 0.02° 2θ

### 2. Differential Scanning Calorimetry (DSC)

Differential scanning calorimetry (DSC) analysis was performed on a sample hermetically sealed in a closed gold or aluminum pan under ambient conditions using a Q2000 (TA Instruments) in the temperature range from −50° C. to 250° C. at a heating rate of 10° C./min. The melting point is understood as the peak maximum.

### 3. Thermogravimetric Analysis (TG-FTIR)

Thermogravimetric analysis (TG-FTIR) was carried out in aluminum sample pans with a pinhole in N2 atmosphere with a Netzsch Thermo-Microbalance TG 209 coupled to a Bruker FT-IR Spectrometer Vector 22 in the temperature range from 20° C. to 360° C. at a heating rate of 10° C./min.

### 4. Dynamic Vapor Sorption (DVS)

Sorption isotherms were obtained using an SPS11-100n Sorptions Prufsystem from ProUmid (formerly Projekt Messtechnik). About 5 mg to about 20 mg of sample were placed into an aluminum sample plan on top of a microbalance and allowed to equilibrate at 50% relative humidity (RH) before starting the following applied measurement program. Humidity change rates of 5% per hour were used. The applied measurement program can be described as follows:
(1) 2 h at 50% RH
(2) 50→0% RH (5%/h); 5 h at 0% RH
(3) 0→95% RH (5%/h); 5 h at 95% RH
(4) 95→0% RH (5%/h); 5 h at 0% RH
(5) 0→95% RH (5%/h); 5 h at 95% RH
(6) 95→50% RH (5%/h); 2 h at 50% RH.

The sample was recovered after completion of the isotherm and re-analyzed by XRPD.

### 4. Classification of Hygroscopicity

Hygroscopicity was classified based on the mass gain at 85% relative humidity (RH) relative to the initial mass as follows: deliquescent (sufficient water adsorbed to form a liquid), very hygroscopic (mass increase of 15%), hygroscopic (mass increase <15% but ≥2%), slightly hygroscopic (mass increase <2% but ≥0.2%), or non-hygroscopic (mass increase <0.2%).

### 5. ¹H-NMR Analysis

¹H-NMR analysis was performed with a Bruker DPX300 spectrometer. ¹H NMR spectra were recorded using proton frequency of 300.13 MHz, 30° excitation pulse, recycle delay of 1 s, accumulation of 16 scans, and deuterated DMSO as the solvent. The chemical shifts were referenced relative to TMS at 0 ppm. The peak at 2.5 ppm corresponds to the solvent peak for DMSO.

Non-limiting embodiments of the present disclosure are set forth below:

Embodiment 1. A crystalline form of a compound of Formula 1,

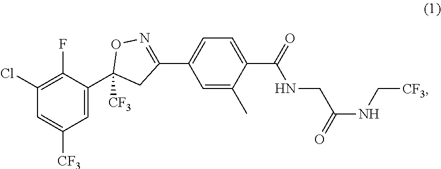 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

characterized in exhibiting an X-ray powder diffraction (XRPD) pattern comprising peaks at 2θ±0.2° values of 18.1°, 19.5°, and 22.3°.

Embodiment 2. The crystalline form of embodiment 1, wherein the XRPD pattern further comprises peaks at one or more 2θ±0.2° values selected from the group consisting of 3.5°, 7.1°, 8.9°, 9.1°, 9.6°, 10.7°, 12.6°, 14.0°, 16.9°, 18.4°, 18.9°, 20.1°, 21.0°, 22.0°, and 22.7°.

Embodiment 3. The crystalline form of embodiment 1, wherein the XRPD pattern further comprises peaks at two or more 2θ±0.2° values selected from the group consisting of 3.5°, 7.1°, 8.9°, 9.1°, 9.6°, 10.7°, 12.6°, 14.0°, 16.9°, 18.4°, 18.9°, 20.1°, 21.0°, 22.0°, and 22.7°.

Embodiment 4. The crystalline form of embodiment 1, wherein the XRPD pattern further comprises peaks at three or more 2θ±0.2° values selected from the group consisting of 3.5°, 7.1°, 8.9°, 9.1°, 9.6°, 10.7°, 12.6°, 14.0°, 16.9°, 18.4°, 18.9°, 20.1°, 21.0°, 22.0°, and 22.7°.

Embodiment 5. The crystalline form of embodiment 1, wherein the XRPD pattern further comprises peaks at four or more 2θ±0.2° values selected from the group consisting of 3.5°, 7.1°, 8.9°, 9.1°, 9.6°, 10.7°, 12.6°, 14.0°, 16.9°, 18.4°, 18.9°, 20.1°, 21.0°, 22.0°, and 22.7°.

Embodiment 6. The crystalline form of embodiment 1, wherein the XRPD pattern further comprises peaks at five or more 2θ±0.2° values selected from the group consisting of 3.5°, 7.1°, 8.9°, 9.1°, 9.6°, 10.7°, 12.6°, 14.0°, 16.9°, 18.4°, 18.9°, 20.1°, 21.0°, 22.0°, and 22.7°.

Embodiment 7. The crystalline form of embodiment 1, wherein the XRPD pattern further comprises peaks at six or more 2θ±0.2° values selected from the group consisting of 3.5°, 7.1°, 8.9°, 9.1°, 9.6°, 10.7°, 12.6°, 14.0°, 16.9°, 18.4°, 18.9°, 20.1°, 21.0°, 22.0°, and 22.7°.

Embodiment 8. The crystalline form of embodiment 1, wherein the peaks have a relative intensity (I/Iₒ) of about 10% or greater.

Embodiment 9. The crystalline form of embodiment 1, wherein said peaks are measured by XRPD using an x-ray wavelength of 1.5406 Å.

Embodiment 10. The crystalline form of any one of embodiments 1-9, wherein each 26 value of the peaks has the angle variation ±0.1°.

Embodiment 11. The crystalline form of any one of embodiments 1-9, having a DSC exothermic peak at a temperature of about 135.5±4° C.

Embodiment 12. The crystalline form of any one of embodiments 1-9, wherein the crystalline form has, in a thermogravimetric analysis (TG-FTIR), a weight loss of 0.4% or less up to about 180° C.

Embodiment 13. The crystalline form of any one of embodiments 1-9, wherein the crystalline form is substantially pure.

Embodiment 14. The crystalline form of embodiment 1, wherein the crystalline form is at least 95% pure.

Embodiment 15. A pharmaceutical composition comprising the crystalline form according to any one of embodiments 1 to 14 as an active ingredient and at least one pharmaceutically acceptable carrier or diluent.

Embodiment 16. The pharmaceutical composition of embodiment 15, wherein the crystalline form makes up 80% or more of a total amount of the compound of Formula 1 in the pharmaceutical composition.

Embodiment 17. The pharmaceutical composition of embodiment 15, wherein the pharmaceutical composition is for treating pests in animals, optionally cats and/or dogs.

Embodiment 18. The pharmaceutical composition of embodiment 15, wherein said pests comprise ticks and/or fleas.

Embodiment 19. A method of preparing the crystalline form according to any one of embodiments 1 to 14, the method comprising steps of:
dissolving, optionally with stirring and/or heating, the compound of Formula 1 in one or more components A, wherein the compound of Formula 1 dissolved in the dissolving step is an amorphous form, one or more crystalline forms, or a combination thereof, and wherein component A is an organic solvent suitable for dissolving the compound of Formula 1, preferably one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; more preferably one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine; and
optionally adding one or more components B, wherein component B is an antisolvent that reduces the solubility of the mixture, wherein component B comprises one or more of water and C₅-C₁₂ cyclic or acyclic hydrocarbon alkanes (e.g., cyclohexane, hexane, heptane, n-heptane, octane, nonane, and decane), preferably one or more of water and heptane.

Embodiment 20. A method of preparing the crystalline form according to any one of embodiments 1 to 14, the method comprising steps of:
dissolving, optionally with stirring and/or heating, the compound of Formula 1 in one or more components A, wherein the compound of Formula 1 dissolved in the dissolving step is an amorphous form, one or more crystalline forms, a combination thereof, and wherein component A is an organic solvent suitable for dissolving the compound of Formula 1, preferably one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; more preferably one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine; and
adding one or more components B, wherein component B is an antisolvent that reduces the solubility of the mixture, component B comprises one or more of water and C₅-C₁₂ cyclic or acyclic hydrocarbon alkanes (e.g., cyclohexane, hexane, heptane, n-heptane, octane, nonane, and decane), preferably one or more of water and heptane;
filtering, washing, and drying the resulting solid.

Embodiment 21. A crystalline form of the compound of Formula 1 prepared by the method according to embodiment 19 or 20.

Embodiment 22. A crystalline form of a compound of Formula 1,

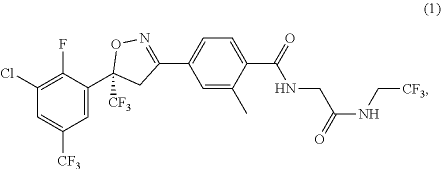 [SMILES: Cc1cc(C2=NO[C@@](c3cc(C(F)(F)F)cc(Cl)c3F)(C(F)(F)F)C2)ccc1C(=O)NCC(=O)NCC(F)(F)F]

characterized in exhibiting an X-ray powder diffraction (XRPD) pattern comprising peaks at 2θ±0.2° values of 4.6°, 20.5°, and 21.7°.

Embodiment 23. The crystalline form of embodiment 22, wherein the XRPD pattern further comprises peaks at one or more 2θ±0.2° values selected from the group consisting of 7.6°, 12.4°, 13.8°, 18.0°, 18.4°, and 20.0°.

Embodiment 24. The crystalline form of embodiment 22, wherein the XRPD pattern further comprises peaks at two or more 2θ±0.2° values selected from the group consisting of 7.6°, 12.4°, 13.8°, 18.0°, 18.4°, and 20.0°.

Embodiment 25. The crystalline form of embodiment 22, wherein the XRPD pattern further comprises peaks at three or more 2θ±0.2° values selected from the group consisting of 7.6°, 12.4°, 13.8°, 18.0°, 18.4°, and 20.0°.

Embodiment 26. The crystalline form of embodiment 22, wherein the XRPD pattern further comprises peaks at four or more 2θ±0.2° values selected from the group consisting of 7.6°, 12.4°, 13.8°, 18.0°, 18.4°, and 20.0°.

Embodiment 27. The crystalline form of embodiment 22, wherein the XRPD pattern further comprises peaks at five or more 2θ±0.2° values selected from the group consisting of 7.6°, 12.4°, 13.8°, 18.0°, 18.4°, and 20.0°.

Embodiment 28. The crystalline form of embodiment 22, wherein the XRPD pattern further comprises peaks at 2θ±0.2° values 7.6°, 12.4°, 13.8°, 18.0°, 18.4°, and 20.0°.

Embodiment 29. The crystalline form of embodiment 22, wherein the peaks have a relative intensity (I/Iₒ) of about 10% or greater.

Embodiment 30. The crystalline form of embodiment 22, wherein said peaks are measured by XRPD using an x-ray wavelength of 1.5406 Å.

Embodiment 31. The crystalline form of any one of embodiments 22-30, wherein each 26 value of the peaks has the angle variation ±0.1°.

Embodiment 32. The crystalline form of any one of embodiments 22-30, having a DSC exothermic peak at a temperature of about 142.8±4° C.

Embodiment 33. The crystalline form of any one of embodiments 22-30, wherein the crystalline form is substantially pure.

Embodiment 34. The crystalline form of embodiment 22, wherein the crystalline form is at least 95% pure.

Embodiment 35. A pharmaceutical composition comprising the crystalline form according to any one of embodiments 22 to 34 as an active ingredient and at least one pharmaceutically acceptable carrier or diluent.

Embodiment 36. The pharmaceutical composition of embodiment 15 or 35, wherein the crystalline form makes up 80% or more of a total amount of the compound of Formula 1 in the pharmaceutical composition.

Embodiment 37. The pharmaceutical composition of embodiment 15 or 35, wherein the pharmaceutical composition is for treating pests in animals, optionally cats and/or dogs.

Embodiment 38. The pharmaceutical composition of embodiment 15 or 35, wherein said pests comprise ticks and/or fleas.

Embodiment 39. A method of preparing the crystalline form according to any one of embodiments 22 to 34, the method comprising steps of:
dissolving, optionally with stirring and/or heating, the compound of Formula 1 in one or more components A, wherein the compound of Formula 1 dissolved in the dissolving step is an amorphous form, one or more crystalline forms, or a combination thereof, and wherein component A is an organic solvent suitable for dissolving the compound of Formula 1, preferably one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; more preferably one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine;
adding one or more components B, wherein component B is an antisolvent that reduces the solubility of the mixture, wherein component B comprises one or more of water and C₅-C₁₂ cyclic or acyclic hydrocarbon alkanes (e.g., cyclohexane, hexane, heptane, n-heptane, octane, nonane, and decane), preferably one or more of water and heptane;
evaporating the one or more components A and the one or more components B; and
filtering the resulting solid.

Embodiment 40. A method of preparing the crystalline form according to any one of embodiments 22 to 34, the method comprising steps of:
dissolving the compound of Formula 1 in one or more components A, wherein the compound of Formula 1 dissolved in the dissolving step is an amorphous form, one or more crystalline forms, or a combination thereof, and wherein component A is an organic solvent suitable for dissolving the compound of Formula 1, preferably one or more of C₁-C₄ alcohol, acetone, acetonitrile, aniline, anisole, benzyl alcohol, butyronitrile, chloroform, cyclohexane, DMSO, ethanol, ethyl acetate, heptane, n-heptane, isopropyl acetate, ethyl lactate, isobutyl acetate, MEK, methanol, medium-chain triglycerides, NMP, 1-octanol, 1-propanol, 2-propanol, TBME, THF, toluene, and triethylamine; more preferably one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine;
adding one or more components B, wherein component B comprises one or more of water and C₅-C₁₂ cyclic or acyclic hydrocarbon alkanes (e.g., cyclohexane, hexane, heptane, n-heptane, octane, nonane, and decane), preferably one or more of water and heptane;
stirring a solution formed from the dissolving step;
evaporating the one or more components A and the one or more components B from the solution;
adding one or more additional component B;
evaporating the one or more components A and the one or more components B from the solution containing one or more additional components B.

Embodiment 41. A crystalline form of the compound of Formula 1 prepared by the method according to embodiment 39 or 40.

### EXAMPLES

### Preparation Example: Preparation of a Compound of Formula 1

A compound of Formula 1, 2-methyl-N-[2-oxo-2-(2,2,2-trifluoroethylamino)ethyl]-4-[(5S)-5-[3-chloro-2-fluoro-5-(trifluoromethyl)phenyl]-5-(trifluoromethyl)-4H-isoxazol-3-yl]benzamide, was prepared according to the process described below.

A mixture of methyl 4-bromo-2-methyl-benzoate (10.0 g, 42.3 mmol), N,N,N′,N′-tetramethylethylene diamine (3.96 mL, 26.3 mmol), palladium (II) acetate (0.5 g, 2.12 mmol), butyldi-1-adamantylphosphine (2 g, 5.29 mmol) and toluene (65 mL) was charged into a pressure vessel. The reaction was pressurized with CO-gas (˜414 kPa) and heated to 85° C. overnight. The reaction was cooled to room temperature. The reaction mixture was filtered through Celite® washing through with toluene and the solvent was removed under reduced pressure. The resulting residue was purified by column chromatography on silica gel (0-10% ethyl acetate in cyclohexane) to obtain methyl 4-formyl-2-methyl-benzoate. LC-MS (method A) Rₜ=0.95 min (no ionization).

A mixture of methyl 4-formyl-2-methyl-benzoate (2.05 g, 11.2 mmol) in MeOH (65 mL) and NaOH in water (2 M, 65 mL) was stirred at room temperature for 5 hours. The reaction mixture was acidified with concentrated HCl until pH˜1. The reaction was diluted with ethyl acetate, the organic layer was separated and the aqueous layer was washed with ethyl acetate. The organic layers were then combined, dried over anhydrous MgSO₄, filtered and concentrated in vacuo to afford 4-formyl-2-methyl-benzoic acid. LC-MS (method B) Rₜ=0.71 min, m/z=163.0 [M−H]−.

At room temperature, DMF (25 μL) was added to a suspension of 4-formyl-2-methyl-benzoic acid (1.8 g, 10.4 mmol) and oxalyl chloride (995 μL, 11.5 mmol) in DCM (35 mL) under N2-atmosphere. The reaction was stirred at room temperature for 3 hours. The reaction mixture was concentrated to afford crude acid chloride. A solution of 2-amino-N-(2,2,2-trifluoroethyl) acetamide-HCl (2.25 g, 11.5 mmol) and NEt₃ (3.2 mL, 23 mmol) in DCM (35 mL) was added to the crude acid chloride at 0° C., the reaction was then warmed to room temperature and stirred for 30 minutes. The reaction was diluted with DCM/water, the organic layer was collected and the solvent was removed under reduced pressure. The crude product was purified by column chromatography on silica gel (0-10% MeOH in DCM) to obtain 4-formyl-2-methyl-N-[2-oxo-2-(2,2,2-trifluoroethylamino)ethyl]benzamide. LC-MS (method A) Rₜ=0.69 min, m/z=303.0 [M+H]⁺.

A NH₂OH-solution (32.6 M in water, 385 μL, 6.28 mmol) was added to 4-formyl-2-methyl-N-[2-oxo-2-(2,2,2-trifluoroethylamino)ethyl]benzamide (1.00 g, 3.14 mmol) in MeOH (15 mL) and the reaction was stirred at room temperature for 6 hours. The solvent was removed under reduced pressure to afford 4-[(E and Z)-hydroxyiminomethyl]-2-methyl-N-[2-oxo-2-(2,2,2-trifluoroethylamino)ethyl]benzamide. LC-MS (method B) Rₜ=0.68 min and 0.70 min, m/z=318.0 [M+H]⁺.

To a solution of 4-[(E and Z)-hydroxyiminomethyl]-2-methyl-N-[2-oxo-2-(2,2,2-trifluoroethylamino)ethyl]benzamide (1.08 g, 3.16 mmol) in DMF (3.34 mL) was added N-chlorosuccinimide (548 mg, 4.10 mmol) and the reaction was heated to 40° C. for 15 min. The reaction was cooled to 0° C. and 1-chloro-2-fluoro-5-(trifluoromethyl)-3-[1-(trifluoromethyl)vinyl]benzene (1.03 g, 3.15 mmol) was added followed by NEt₃ (484 μL, 3.47 mmol). The reaction was stirred at room temperature. The reaction was diluted with ethyl acetate and brine. The organic layer was separated and was washed with more brine, dried over anhydrous MgSO₄, filtered and concentrated under reduced pressure. The crude product was purified by column chromatography on silica gel (0-60% ethyl acetate in cyclohexane) to afford the title compound and its R-enantiomer. LC-MS (method A) Rₜ=1.38 min, m/z=608.0 [M+H]+. ¹H NMR (CDCl₃, 400 MHz) δ 8.04 (dd, J=2, 6 Hz, 1H), 7.81 (dd, J=2, 6 Hz, 1H), 7.47-7.56 (m, 3H), 6.90 (br s, 1H), 6.71 (br s, 1H), 4.18-4.23 (m, 3H), 3.84-4.00 (m, 3H), 2.48 (s, 3H).

The two enantiomers were separated by SFC on Chiralpak® AS-H with column dimensions of 250 mm×30 mm (5 μm), a flow rate of 152 ml/min, and a CO₂-based mobile phase with 10% MeOH containing 0.2% N,N-dimethylethylamine as additive to give the compound of Formula 1 (2-methyl-N-[2-oxo-2-(2,2,2-trifluoroethylamino)ethyl]-4-[(5S)-5-[3-chloro-2-fluoro-5-(trifluoromethyl)phenyl]-5-(trifluoromethyl)-4H-isoxazol-3-yl]benzamide), and its R-enantiomer, 2-methyl-N-[2-oxo-2-(2,2,2-trifluoroethylamino)ethyl]-4-[(5R)-5-[3-chloro-2-fluoro-5-(trifluoromethyl)phenyl]-5-(trifluoromethyl)-4H-isoxazol-3-yl]benzamide.

### Preparation of Crystalline Form a of Compound of Formula 1

### Example 1: Preparation of Crystalline Form A of Compound of Formula 1

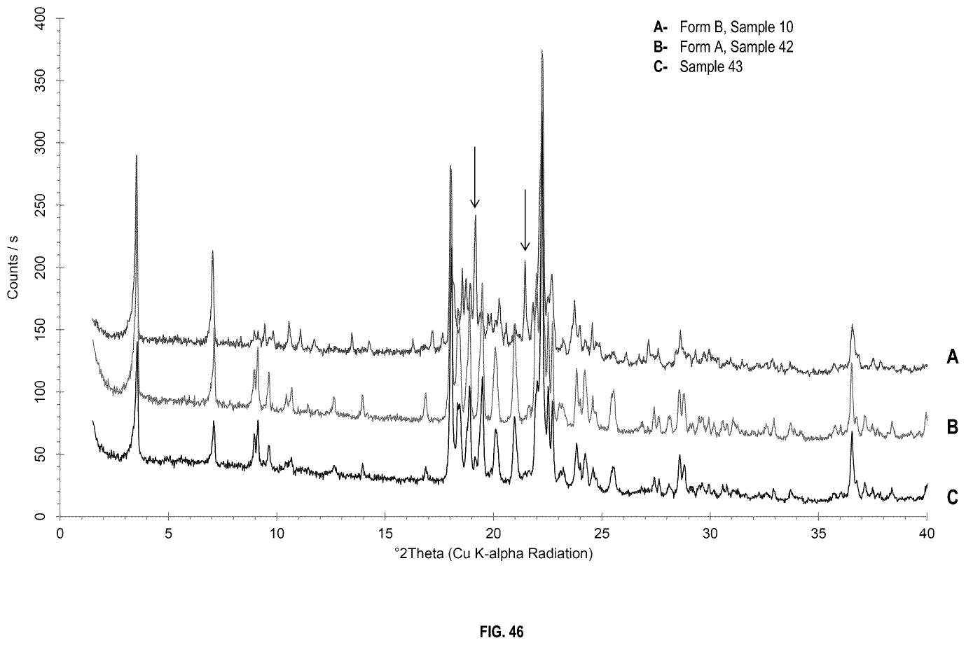

At room temperature, 15 mL of an ethanol/water 2:1 mixture was added to 5.0327 g of the compound of Formula 1 after separation from its R-enantiomer in the Preparation Example (above). An orange paste formed around the magnetic bar. After 5 minutes of stirring, a suspension formed, and the sticky material around the magnetic bar became solid. A spatula was used to break the agglomerates, and 15 mL of the solvent mixture was added. After two hours of stirring, a thick suspension was observed and 10 mL of the solvent system was added. Agglomerates were no longer observed. After one night of stirring at room temperature, the suspension was filtered over fritted glass (porosity 4). The wet cake was white, and the mother liquor was slightly orange-colored. The mother liquor was used to rinse the reactor. 10 mL of the ethanol/water 2:1 mixture was used to wash the cake. The filter cake was dried on the filter for 10 minutes with applied vacuum and then transferred (8.141 g) to a recipient for further drying under vacuum (<5 mbar) at room temperature. After overnight drying, 4.4091 g of material was recovered and submitted for XRPD analysis (see FIGS. 46, 47, 53, 64, 75, 76). Yield: 88%.

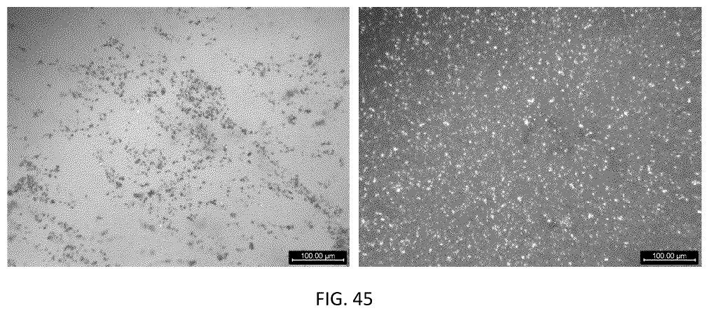

Crystalline Form A of Example 1 is herein referred to as Sample 42. Light microscopy images for Sample 42 appear in FIG. 45.

### Example 2: Preparation of Crystalline Form A of Compound of Formula 1

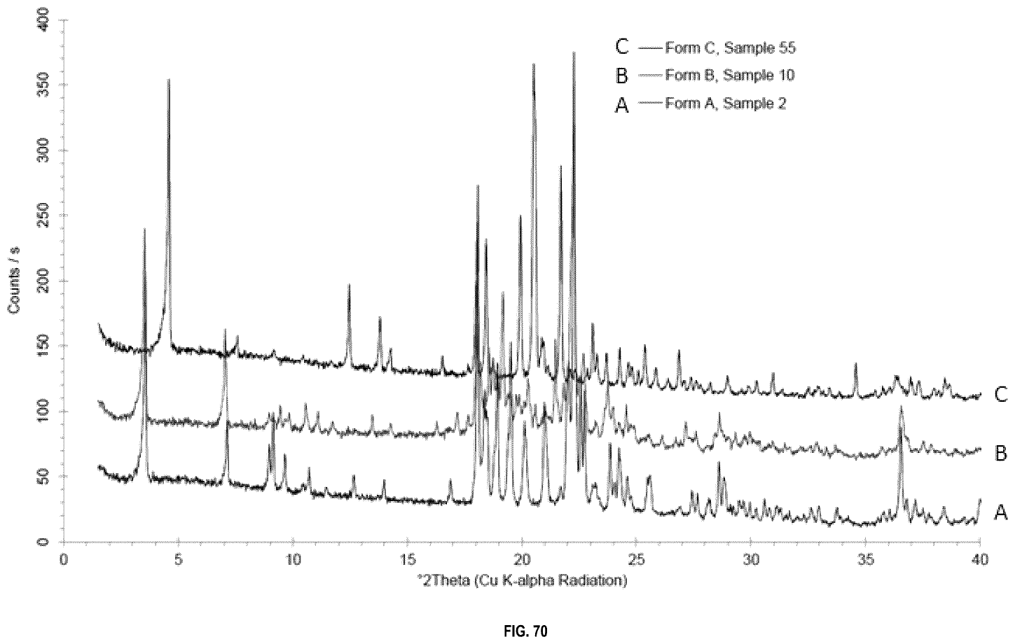

200 μL of an ethanol/water 2:1 mixture was added to 90.2 mg of the amorphous form of the compound of Formula 1. A solution was obtained under stirring at room temperature. After 5 minutes, precipitation was observed, and stirring was no longer possible. An additional 600 μL of the solvent mixture was added, and further stirring was conducted at room temperature. After three days of stirring, the obtained colorless suspension was filtered using a centrifugal unit filter (PVDF, 0.22 μm, 5 min, 5000 rpm). The vial was flushed with the mother liquor. The recovered powder was submitted for XRPD. Crystalline Form A of Example 2 is herein referred to as Sample 2 (see FIGS. 13 and 70).

### Example 2a: Preparation of Crystalline Form A of Compound of Formula 1

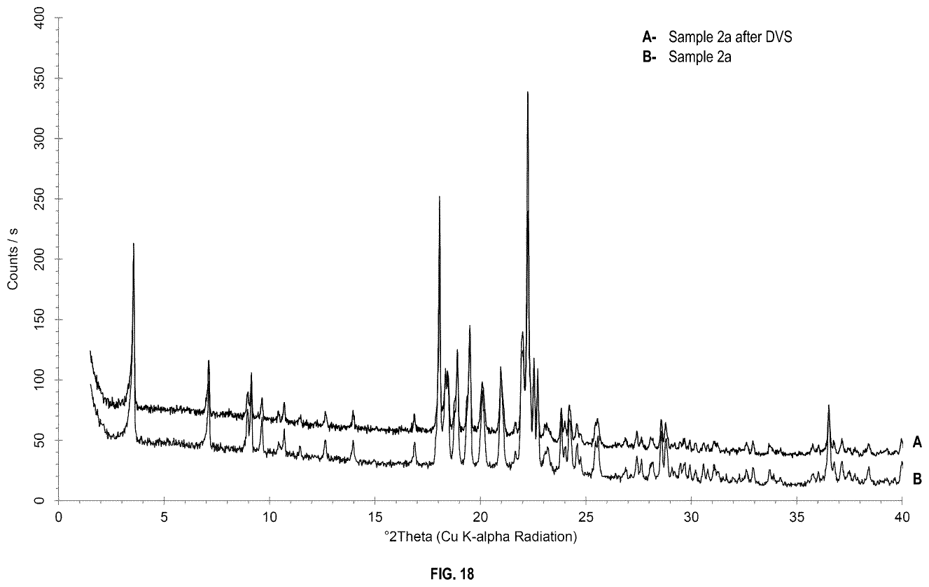

The remainder of Sample 2 from Example 2 was dried overnight at room temperature, 5 mbar. This dried sample is herein referred to as Sample 2a. There was no change in the XRPD pattern, which corresponded to Crystalline Form A of the compound of Formula 1 (see FIGS. 18 and 29). Thermogravimetric analysis (TG-FTIR) showed a loss of 0.13% of water from 25 to 180° C. (see FIG. 10). DSC analysis indicated a melting point at 135.5° C., onset at 132.7° C. with enthalpy of fusion at 65.6 J/g (see FIG. 15). DVS analysis showed 0.5% absorption of water at 95% relative humidity for five hours (see FIGS. 16 and 17). The XRPD pattern after DVS also corresponded to Crystalline Form A of the compound of Formula 1 (see FIG. 18).

### Example 3: Preparation of Crystalline Form A of Compound of Formula 1

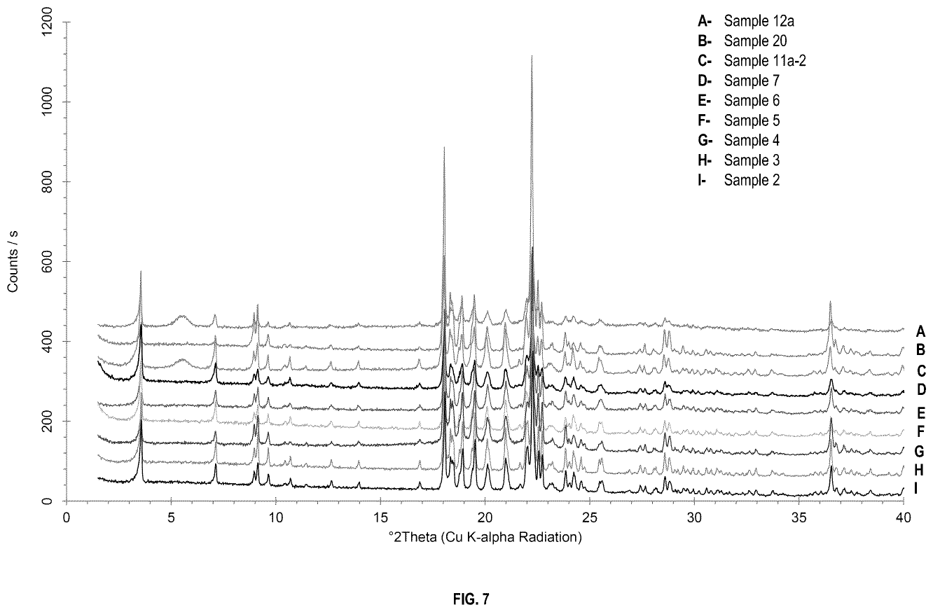

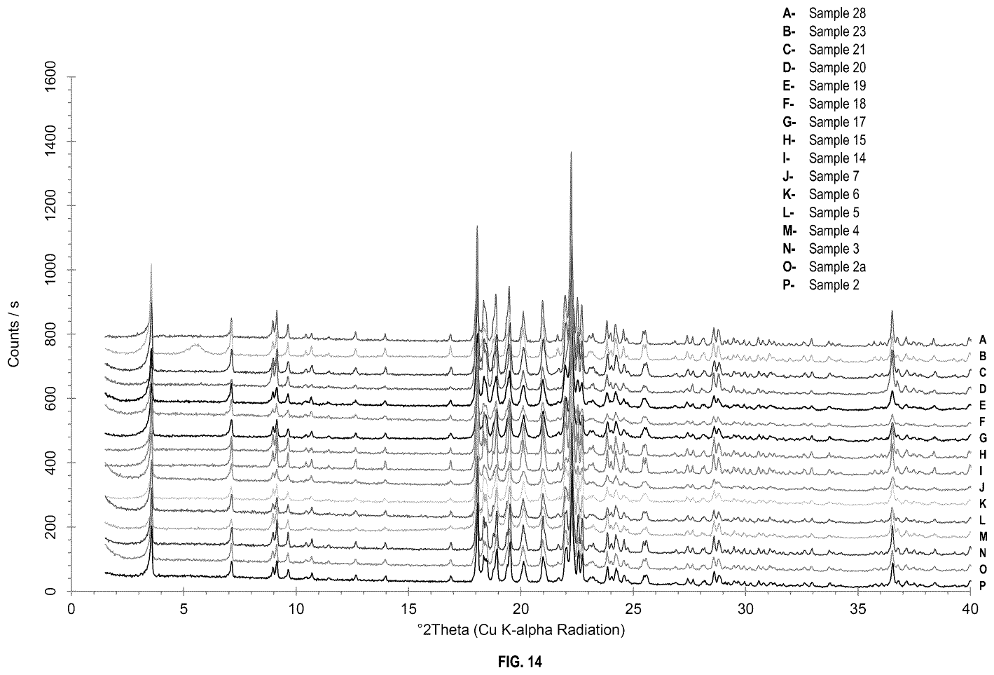

200 μL of a 2-propanol/water 3:1 mixture was added to 80.1 mg of the amorphous form of the compound of Formula 1. A solution was obtained under stirring at room temperature. After 10 minutes, precipitation was observed. An additional 400 μL of the solvent mixture was added, and further stirring was conducted at room temperature. After three days of stirring, the colorless suspension was filtered using a centrifugal unit filter (PVDF, 0.22 μm, 5 min, 5000 rpm). The vial was flushed with the mother liquor. The recovered powder was submitted for XRPD (see FIGS. 7 and 14). Crystalline Form A of Example 3 is herein referred to as Sample 3.

### Example 4: Preparation of Crystalline Form A of Compound of Formula 1

200 μL of an ethyl acetate/heptane 1:1 mixture was added to 80.4 mg of the amorphous form of the compound of Formula 1. A solution was obtained under stirring at room temperature. After three days, a solution was still observed. 500 μL of heptane was slowly added, and a cloudy solution with some sticky material was formed. After 10 minutes, a suspension started to form. After 5 hours of stirring at room temperature, the suspension was filtered using a centrifugal unit filter (PVDF, 0.22 μm, 5 min, 5000 rpm). The vial was flushed with the mother liquor. The recovered powder was submitted for XRPD (see FIGS. 7 and 14). Crystalline Form A of Example 4 is herein referred to as Sample 4.

### Example 5: Preparation of Crystalline Form A of Compound of Formula 1

78.5 mg of the amorphous form of the compound of Formula 1 was dissolved in 500 μL of acetonitrile at room temperature. 500 μL of water was added dropwise under stirring. After the addition, some “oily drops” were observed in the solution. An additional 1 mL of water was added (acetonitrile to water 1:3), and a cloudy solution with some oily drops was obtained. Further stirring at room temperature occurred. After three days of stirring, a colorless suspension was obtained and filtered using a centrifugal unit filter (PVDF, 0.22 μm, 5 min, 5000 rpm). The vial was flushed with the mother liquor. The recovered powder was submitted for XRPD (see FIGS. 7 and 14). Crystalline Form A of Example 5 is herein referred to as Sample 5.

### Example 6: Preparation of Crystalline Form A of Compound of Formula 1

91.1 mg of the amorphous form of the compound of Formula 1 was dissolved in 500 μL of 1-propanol at room temperature. 500 μL of water was added dropwise under stirring. After the addition a cloudy solution was observed. An additional 1 mL of water was added (1-propanol to water 1:3), but no precipitation was observed. Further stirring at room temperature occurred. After three days of stirring, a colorless suspension was obtained and filtered using a centrifugal unit filter (PVDF, 0.22 μm, 5 min, 5000 rpm). The vial was flushed with the mother liquor. The recovered powder was submitted for XRPD (see FIGS. 7 and 14). Crystalline Form A of Example 6 is herein referred to as Sample 6.

### Example 7: Preparation of Crystalline Form A of Compound of Formula 1

81.8 mg of the amorphous form of the compound of Formula 1 was dissolved in 500 μL of TBME at room temperature. 500 μL of heptane was added dropwise under stirring. After the addition, a solution was still obtained, and an additional 500 μL of heptane was added; a cloudy solution was obtained. An additional 500 μL of heptane was added, and a solution with sticky material formed and was submitted to vortex and sonication treatment for 2 minutes. No change was observed, and further stirring was conducted at room temperature. After three days of stirring, a colorless suspension was obtained and filtered using a centrifugal unit filter (PVDF, 0.22 μm, 5 min, 5000 rpm). The vial was rinsed with the mother liquor. The recovered powder was submitted for XRPD (see FIGS. 7 and 14). Crystalline Form A of Example 7 is herein referred to as Sample 7.

### Example 8: Preparation of Crystalline Form B of Compound of Formula 1

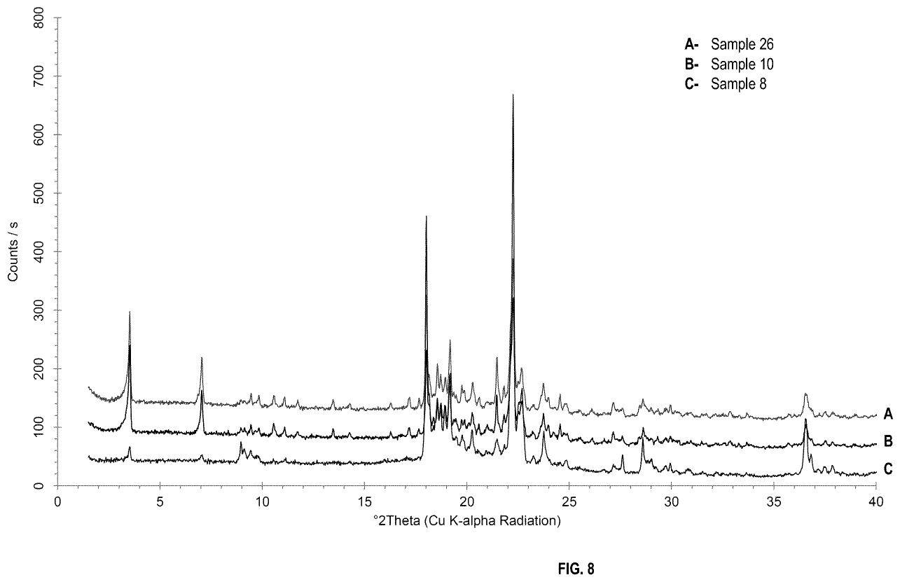

83.8 mg of the amorphous form of the compound of Formula 1 was dissolved in 500 μL of MEK at room temperature. 500 μL of heptane was added dropwise under stirring. No precipitation was observed, and an additional 1 mL of heptane was added (MEK to heptane ratio 1:3). A solution was still observed and further stirring was conducted at room temperature. After three days of stirring, no precipitation was observed, and the vial was opened to allow solvent evaporation. After one day, ⅓ of the solvent was evaporated and precipitation was observed. The resulting suspension was filtered using a centrifugal unit filter (PVDF, 0.22 μm, 5 min, 5000 rpm). The vial was rinsed with the mother liquor. The recovered powder was submitted for XRPD (see FIG. 8). Crystalline Form B of Example 8 is herein referred to as Sample 8.

### Example 9: Preparation of Crystalline Forms A and B of Compound of Formula 1

92.9 mg of the amorphous form of the compound of Formula 1 was dissolved in 500 μL of THF at room temperature. The vial was opened to allow solvent evaporation at room temperature. After three days, an oily residue was obtained. Further evaporation was conducted under nitrogen flow at room temperature. After 4 days, the sample was still an oily residue. 400 μL of isobutyl acetate was added to the oily residue. After stirring, a solution was obtained at room temperature. 1.2 mL of heptane (3 times 400 μL steps) was slowly added, but precipitation was not observed. The solution was seeded with Samples 6 and 10; seed crystals did not dissolve. After 30 minutes of stirring, a suspension was obtained, and half of it was filtered (centrifugal unit filter PVDF, 0.22 μm, 5 min, 5000 rpm). The recovered powder was designated as Sample 11a-1 and was submitted for XRPD (Kapton foils). The rest of the suspension was further stirred at room temperature. After three days of stirring, filtration was conducted using a centrifugal unit filter (PTFE, 0.22 μm, 5 min, 5000 rpm). The recovered powder was designated as Sample 11a-2 and was submitted for XRPD (Kapton foils) (see FIG. 7).

### Example 10: Preparation of Crystalline Form A of Compound of Formula 1

81.8 mg of the amorphous form of the compound of Formula 1 was dissolved in 500 μL of acetonitrile at room temperature. The vial was opened to allow solvent evaporation at room temperature. After three days, an oily residue was obtained. Further evaporation was conducted under nitrogen flow at room temperature. After 4 days, the sample was still an oily residue. 400 μL of NMP was added to the oily residue. After stirring, a solution was obtained at room temperature. 800 μL of water (2 times 400 μL steps) was slowly added, and precipitation was observed. A cloudy solution formed with sticky material around the stirrer. After vortex treatment, a suspension could be obtained, and filtration was conducted using a centrifugal unit filter (PTFE, 0.22 μm, 5 min, 5000 rpm). However, only a small amount of sticky material was present on the filter. The mixture was recovered and further stirred at room temperature. After three days of stirring, a suspension was obtained. Filtration was conducted using centrifugal unit filter (PTFE, 0.22 μm, 5 min, 5000 rpm). The recovered powder was designated as Sample 12a and was submitted for XRPD (Kapton foils) (see FIG. 7).

### Example 11: Preparation of Crystalline Form A+B of Compound of Formula 1

74.6 mg of the amorphous form of the compound of Formula 1 was suspended in 1 mL of water at 75° C. After 5 minutes of stirring, a sticky material formed around the stirrer bar. After one night of stirring, an agglomerate was still observed around the stirrer and was scraped off with a spatula. Further stirring was conducted for 5 hours at 75° C. and then the suspension was filtered using a centrifugal unit filter (PVDF, 0.22 μm, 5 min, 5000 rpm). The vial was flushed with the mother liquor. The recovered powder was designated Sample 13 and was submitted for XRPD. The XRPD pattern corresponded to Form A with some small additional reflections (17.2°, 19.2°, and 21.5° 2θ) that can be attributed to Form B. The remainder of Sample 13 was dried overnight at room temperature and 5 mbar. This dried remainder was designated Sample 13a and submitted for XRPD, which showed no change from the XRPD pattern of Sample 13. Thermogravimetric analysis (TG-FTIR) indicated a weight loss of 0.74% of water from 25 to 170° C. (see FIG. 12).

### Example 12: Preparation of Crystalline Form A of Compound of Formula 1

83 mg of the amorphous form of the compound of Formula 1 was suspended in 0.5 mL of an ethanol/water 1:1 mixture at 75° C. After 5 minutes stirring, an emulsion with oily drops formed. After overnight stirring at 75° C., a suspension formed, and filtration was conducted using centrifugal unit filter (PVDF, 0.22 μm, 5 min, 5000 rpm). The recovered powder was designated Sample 14 and was submitted for XRPD (see FIG. 14).

### Example 13: Preparation of Crystalline Form A of Compound of Formula 1

73.8 mg of the amorphous form of the compound of Formula 1 was suspended in 0.5 mL of methanol/water 2:1 at 60° C. A fine suspension was observed after 10 minutes of stirring. After overnight stirring at 60° C., a suspension formed and filtration was conducted using a centrifugal unit filter (PVDF, 0.22 μm, 5 min, 5000 rpm). The recovered powder was designated Sample 15 and was submitted for XRPD (see FIG. 14).

### Example 14: Preparation of Crystalline Form A of Compound of Formula 1

39.8 mg of Sample 5 and 58.7 mg of Sample 6 were suspended in 1 mL of an ethanol/water 1:9 mixture at room temperature. After vortex treatment and 10 minutes stirring, seeding was conducted with Sample 10. An additional 1 mL of the solvent mixture was added and further stirred at room temperature. After three days of stirring, the suspension was filtered using centrifugal unit filter (PVDF, 0.22 μm, 5 min, 5000 rpm). The vial was flushed with the mother liquor. The recovered powder was designated Sample 17 and was submitted for XRPD (see FIGS. 14 and 70).

### Example 15: Preparation of Crystalline Form A of Compound of Formula 1

85 mg of the amorphous form of the compound of Formula 1 was suspended in 200 μL of an acetone/water 1:2 mixture at room temperature. Sticky material formed but not completely dissolved. After vortex treatment, a cloudy solution was obtained. After two hours of stirring, a very thick suspension formed, and 1 mL of the acetone/water mixture was added. After three days of stirring, the suspension was filtered using centrifugal unit filter (PVDF, 0.22 μm, 5 min, 5000 rpm). The vial was flushed with the mother liquor. The recovered powder was designated Sample 18 and was submitted for XRPD, which corresponded to Crystalline Form A (see FIG. 14).

### Example 16: Preparation of Crystalline Form A of Compound of Formula 1

87.4 mg of the amorphous form of the compound of Formula 1 was suspended in 200 μL of a methanol/water 2:1 mixture at room temperature. Sticky material formed but did not completely dissolve. After two hours of stirring, a very thick suspension formed, and 1 mL of the methanol/water mixture was added. After three days of stirring, the suspension was filtered using a centrifugal unit filter (PVDF, 0.22 μm, 5 min, 5000 rpm). The vial was flushed with the mother liquor. The recovered powder was designated Sample 19 and was submitted for XRPD, which corresponded to Crystalline Form A (see FIG. 14).

### Example 17: Preparation of Crystalline Form A of Compound of Formula 1

72 mg of the amorphous form of the compound of Formula 1 was suspended in 200 μL of a 2-propanol/heptane 1:1 mixture at room temperature. A cloudy solution with oily drops formed. After two hours of stirring, a very thick suspension formed, and 0.5 mL of the 1-propanol/water mixture was added. A solution was obtained and further stirred at room temperature. After three days, no suspension formed, and the solution was further stirred at 5° C. After three days of stirring, a suspension formed at 5° C.; filtration was conducted using a centrifugal unit filter (PTFE, 0.22 μm, 5 min, 5000 rpm, 5° C.). The wet filter cake was designated Sample 20 and was submitted for XRPD, which corresponded to Crystalline Form A (see FIGS. 7 and 14).

### Example 18: Preparation of Crystalline Form A of Compound of Formula 1

79 mg of the amorphous form of the compound of Formula 1 was suspended in 200 μL of a 1-propanol/water 1:1 mixture at room temperature. A cloudy solution with oily drops formed. After two hours of stirring, a very thick suspension formed and 1 mL of the 1-propanol/water mixture was added. After three days of stirring, the suspension was filtered using a centrifugal unit filter (PVDF, 0.22 μm, 5 min, 5000 rpm). The vial was flushed with the mother liquor. The recovered powder was designated Sample 21 and was submitted for XRPD, which corresponded to Crystalline Form A (see FIG. 14).

### Example 19: Preparation of Crystalline Form A of Compound of Formula 1

41.8 mg of Sample 18 and 31.6 mg of Sample 19 (total of 73.4 mg, Form A) were suspended in 0.6 mL of a heptane/acetone 9:1 mixture at room temperature. After 5 minutes of stirring, the suspension was seeded with Sample 10 (Form B). After 5 days of stirring at room temperature, the suspension was filtered using a centrifugal unit filter (PTFE, 0.22 μm, 5 min, 5000 rpm). The vial was flushed with the mother liquor. The recovered wet powder was designated Sample 23 and was submitted for XRPD, which corresponded to Crystalline Form A (see FIG. 14).

### Example 20: Preparation of Crystalline Form B of Compound of Formula 1

97 mg of the amorphous form of the compound of Formula 1 was dissolved in 500 μL of TBME at room temperature. The vial was opened to allow solvent evaporation. After 4 days, a dried residue was obtained and scraped out with a spatula. The obtained powder was designated Sample 26 and was submitted for XRPD, which corresponded to Crystalline Form B (see FIG. 8).

### Example 21: Preparation of Crystalline Form A of Compound of Formula 1

500 μL of methanol/water 1:1 mixture was added to 101.2 mg of the amorphous form of the compound of Formula 1. Sticky material formed, and the mixture was heated to 60° C. After 10 min of stirring, the sticky material transformed into a white solid, and 200 μL of methanol was added. No change was observed. After vortex treatment, a suspension was obtained and further stirred at 60° C. After 4 days of stirring, a solution with material on the glass side was obtained. The mixture was submitted to vortex treatment, and a suspension was obtained. After additional 5 hours stirring, the suspension was filtered using centrifugal unit filter (PTFE, 0.22 μm, 5 min, 5000 rpm). The vial was flushed with the mother liquor. The recovered wet powder was designated Sample 28 and was submitted for XRPD, which corresponded to Crystalline Form A (see FIG. 14).

### Example 22: Preparation of Crystalline Form B of Compound of Formula 1

500 μL of acetone/heptane 1:1 mixture was added to 99.4 mg of the amorphous form of the compound of Formula 1, and the mixture was heated to 60° C. A solution was obtained at 60° C. Then the heating was stopped, and the temperature was allowed to decrease to room temperature. At room temperature, a solution was still observed and was further stirred at 5° C. After 4 days stirring at 5° C., no precipitation was observed and the vial was placed in the freezer (−26° C.) overnight. However, no precipitation took place, and 0.5 mL of heptane was added and an acetone/heptane 1:3 ratio was reached. No precipitation was observed and further stirring was conducted at 5° C. No change was observed after 5 hours stirring at 5° C. Therefore the solution was seeded with Sample 2 (Form A) and Sample 10 (Form B). After overnight stirring at 5° C., a solution was still observed. 0.5 mL of heptane was slowly added at room temperature, and an acetone/heptane 1:5 ratio was reached. No precipitation was observed; the vial was opened to allow solvent evaporation under stirring. After 2 hours, only a small amount had evaporated, and a suspension was obtained. Filtration was conducted using a centrifugal unit filter (PTFE, 0.22 μm, 5 min, 5000 rpm). The vial was flushed with the mother liquor. The recovered wet powder was designated Sample 30 and was submitted for XRPD, which corresponded to Crystalline Form B. The remainder of Sample 30 was dried two days under vacuum (<5 mbar) at room temperature. The dried remainder was designated Sample 30a. DSC analysis of Sample 30a indicated a melting peak at 131° C., onset at 124.8° C. with an enthalpy of 58.2 J/g (see FIG. 21).

### Example 22: Preparation of Crystalline Form A of Compound of Formula 1

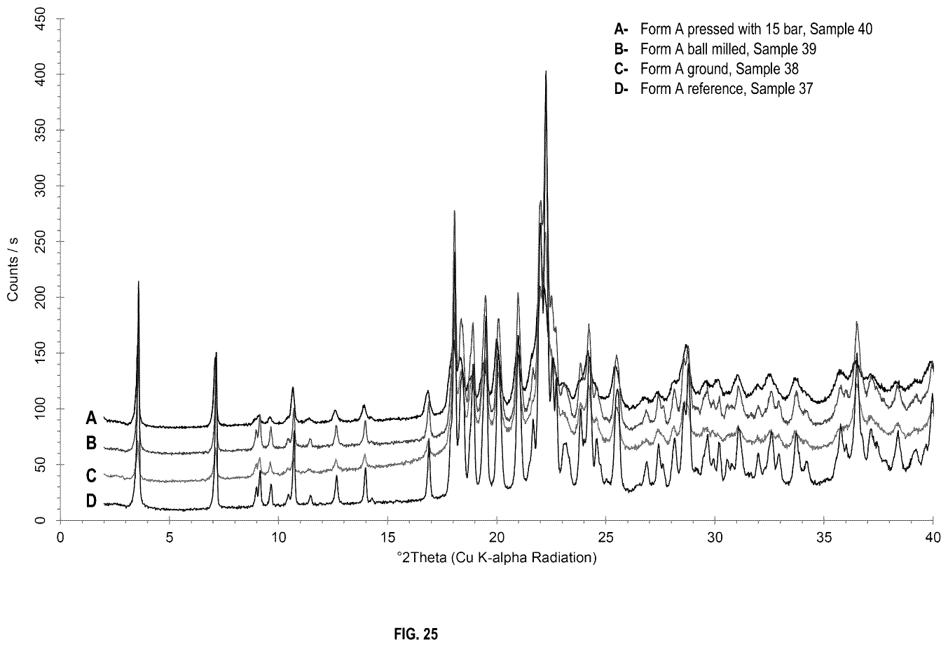

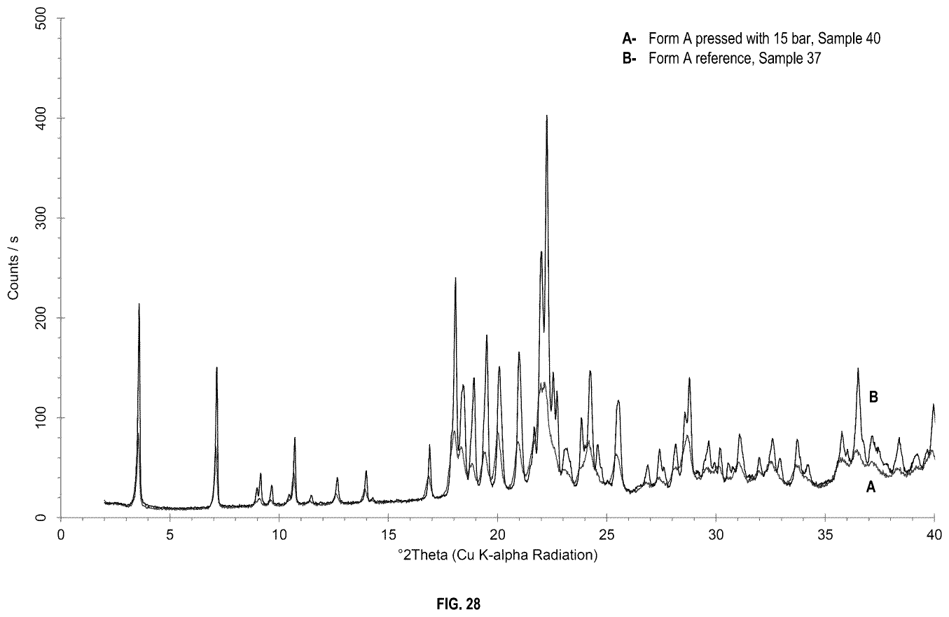

1 mL of an ethanol/water 2:1 mixture was added to 413.5 mg of the amorphous form of the compound of Formula 1. After two minutes of stirring at room temperature, a solution with sticky material was observed. After 30 minutes of stirring, a very thick suspension was observed, and 2 mL of the solvent mixture was added. After three days of stirring at room temperature, the suspension was filtered using a centrifugal unit filter (PVDF, 0.22 μm, 5000 rpm, 5 min). The vial was rinsed with the mother liquor. The recovered powder, which was designated Sample 37, was placed in a vial and dried overnight at room temperature and 5 mbar vacuum and then submitted for XRPD, which corresponded to Crystalline Form A (see FIGS. 25-28).

### Example 23a: Grinding Experiment

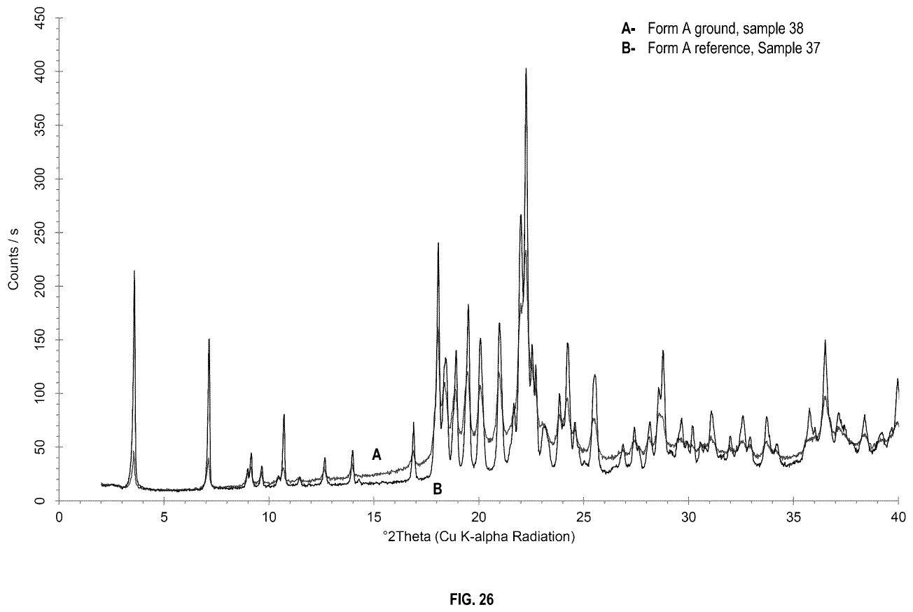

75 mg of Sample 37 was placed in a mortar, and grinding was conducted with a pestle. Three 1-minute grinding steps were conducted, and between each step, the powder was gathered together in the middle with a spatula. At the end of the experiment, the powder, which was designated Sample 38, was scraped off with a spatula and was submitted for XRPD (see FIGS. 25 and 26), which corresponded to Form A but with broader and less intense reflections, suggesting loss of crystallinity. An increase of the baseline suggests amorphization.

### Example 23b: Ball-Milling Experiment

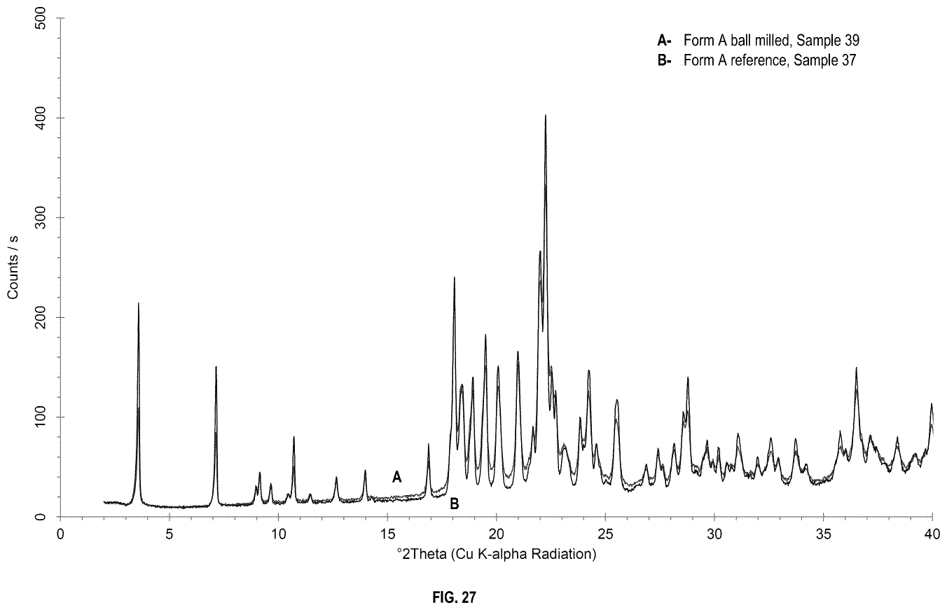

75 mg of Sample 37 was placed in a container with two 3-mm diameter milling balls. Three milling steps of 5 minutes each were conducted with a frequency of 30 s⁻¹. The powder, which was designated Sample 39, stuck to the edge of the container and was scraped off with a spatula, and was submitted for XRPD, which corresponded to Crystalline Form A but with slightly reduced peak intensity (see FIGS. 25 and 27).

### Example 23c: IR-press Experiment

Approx. 100 mg of Sample 37 was placed between two plungers (metal cylinders) in the IR press die set. The sample was pressed with the IR press for 5 minutes with 15 bar. The recovered powder, which was designated Sample 40, was scraped off from the cylinder with a spatula, and was submitted for XRPD (see FIGS. 25 and 38), which corresponded to Form A but with broader and less intense peaks, suggesting loss of crystallinity. An increase of the baseline suggests amorphization.

### Example 24a: Preparation of Crystalline Forms A and B of Compound of Formula 1

1.1714 g of amorphous form of the compound of Formula 1 was placed in a 25-mL reactor with a magnetic stirring bar in the EasyMax 102 device. 15 mL of 2-propanol/water 1:1 mixture was added to the powder. A sticky material formed on the bottom of the vial. A turbidity probe was placed in the reactor. Stirring was started at 500 rpm; however, the magnetic bar was blocked by the sticky material. A spatula was used to “break” the block. Suspension formed after 2 minutes of stirring. Heating was started to 60° C. with 1 K/minute. At 60° C., a fine suspension was obtained. 2 mL of the 2-propanol/water 1:1 mixture was added and temperature was increased to 65° C. A solution was obtained at 65° C., and cooling was started with 0.2 K/hour to 5° C. After overnight stirring, the cooling rate was changed to 5 K/hour. The obtained suspension was further stirred at 5° C. for 8 hours. It was observed that the stirring stopped because the magnetic bar was blocked by the thick suspension. Filtration was conducted over fritted glass filter (porosity 4). The reactor was rinsed with 3 mL of the mother liquor. The cake was dried on the filter with applied vacuum for approx. 30 minutes. 1.4266 g of white powder was recovered. Then, further drying was conducted under vacuum (<5 mbar) at room temperature. 1.0196 g of powder was recovered after overnight drying. Yield: 87%

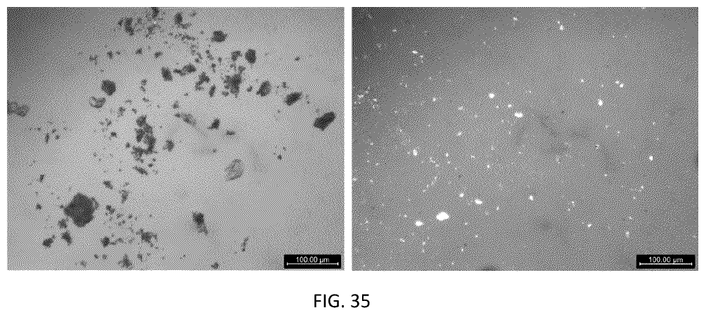

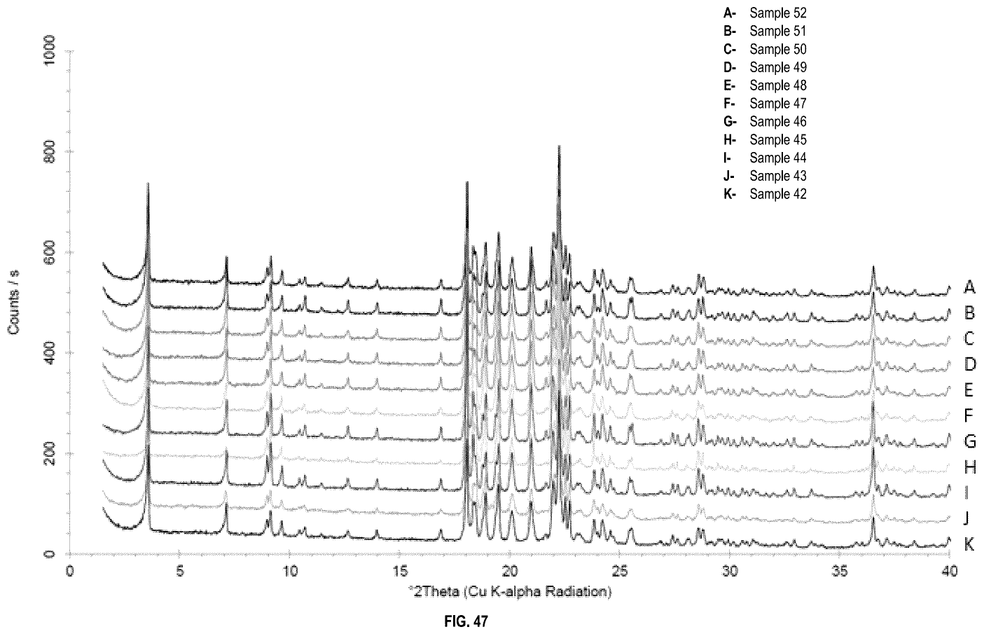

XRPD pattern (see FIGS. 46, 47) corresponds to Form A with small amount of Form B (visible at 19.2° and 21.5° 2θ). Under light microscopy, small crystals were observed; shape could not be determined (see FIG. 35). Crystalline Forms A and B of Example 24a are herein referred to as Sample 43.

### Example 24b: Preparation of Crystalline Form A of Compound of Formula 1

1.1415 g of amorphous form of the compound of Formula 1 was placed in a 25-mL reactor with a magnetic stirring bar in the EasyMax 102 device. 18 mL of ethyl acetate/heptane 1:3 mixture was added to the powder. A sticky material formed on the bottom of the vial. A turbidity probe was placed in the reactor. Stirring was started at 500 rpm; however, the magnetic bar was blocked by the sticky material. A spatula was used to “break” the block. A suspension formed after 2 minutes of stirring. Heating was started to 60° C. with 1 K/minute. At 60° C., a suspension was obtained and cooling was started with 0.2 K/hour to 5° C. After overnight stirring, the cooling rate was changed to 5 K/hour. The obtained suspension was further stirred at 5° C. for 9 hours. It was observed that the stirring had stopped because the magnetic bar was blocked by the thick suspension. Filtration was conducted over fritted glass filter (porosity 4). The reactor was rinsed with 3 mL of the mother liquor. The cake was dried on the filter with applied vacuum for approx. 30 minutes. 694.2 mg of white powder was recovered. Then, further drying was conducted under vacuum (<5 mbar) at room temperature. 692.1 mg of powder was recovered after overnight drying. Yield: 61%

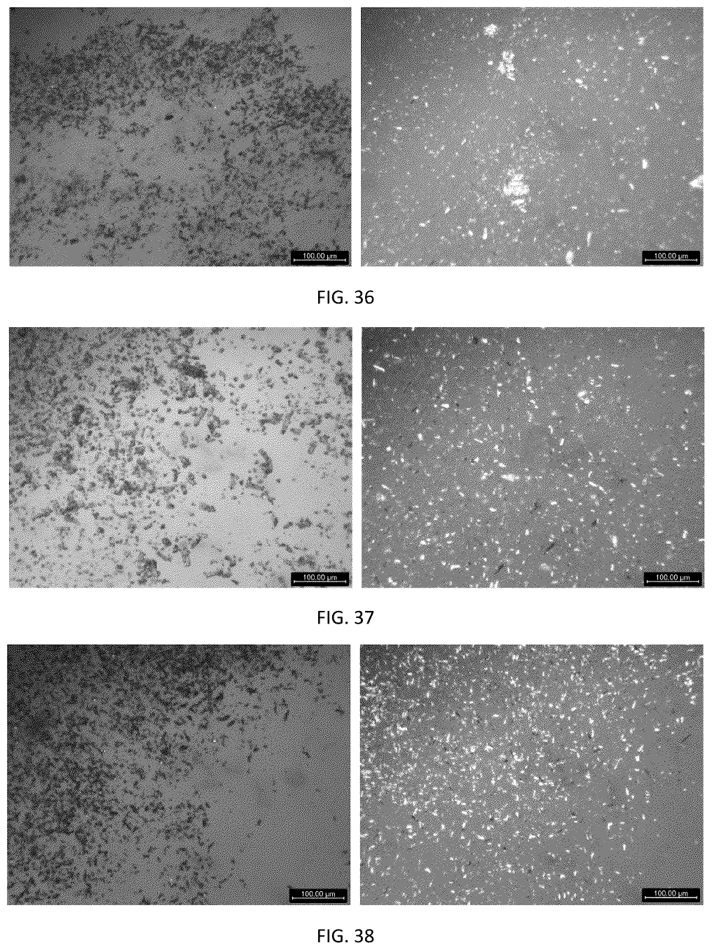

XRPD pattern corresponds to Form A (see FIG. 51). Under light microscopy, small crystals (needle/rod shape) were observed (see FIG. 36). Crystalline Form A of Example 24b is herein referred to as Sample 44.

### Example 24c: Preparation of Crystalline Form A of Compound of Formula 1

1.0314 g of amorphous form of the compound of Formula 1 was placed in a 25-mL reactor with a magnetic stirring bar in the EasyMax 102 device. 9 mL of 2-propanol was added to the powder. Stirring was started at 500 rpm, and most of the material dissolved; but some sticky material formed. Heating was started up to 60° C. at 1 K/minute. At 60° C., clear solution was obtained and sticky material was no longer observed. 9 mL of water was added with 0.5 mL/min. After the addition of about 7 mL of water, local precipitation was observed but did not persist. After the addition, a solution was observed at 60° C. Cooling was started to 23° C. with 5 K/hour. At approx. 43° C., a cloudy solution was observed and was seeded with approx. 5 mg of Sample 42. Seed crystals did not dissolve. At 42° C., a thick suspension formed. After overnight stirring at 23° C., a thick suspension was obtained, and stirring was difficult; therefore, the stirring speed was increased to 1000 rpm. After one hour of stirring, the suspension was filtered over a fritted glass filter (porosity 4). The reactor was rinsed with 3 mL of the mother liquor. The cake was dried on the filter with applied vacuum for approx. 30 minutes. 893.2 mg of white powder was recovered. Then, further drying was conducted under vacuum (<5 mbar) at room temperature. 889.9 mg of powder was recovered after overnight drying. Yield: 86.3%.

XRPD pattern corresponds to Form A (see FIG. 47). Under light microscopy, small crystals (needle/rod shape) were observed (see FIG. 37). Crystalline Form A of Example 24c is herein referred to as Sample 45.

### Example 24d: Preparation of Crystalline Form A of Compound of Formula 1

1.0063 g of amorphous form of the compound of Formula 1 was placed in a 25-mL reactor with a magnetic stirring bar in the EasyMax 102 device. 4 mL of ethyl acetate was added to the powder. Most of the material dissolved, but some sticky material formed. Stirring was started at 500 rpm, and all the material dissolved. Heating was started up to 60° C. with 1 K/minute. At 60° C., clear solution was obtained and 16 mL of heptane was added with 0.5 mL/min. After the addition, solution was observed at 60° C. Cooling was then started to 23° C. with 5 K/hour. At 47° C., the solution was seeded with approx. 5 mg of Sample 42, and a cloudy solution formed. After overnight stirring at 23° C., the suspension was filtered over a fritted glass filter (porosity 4). The reactor was rinsed with 3 mL of the mother liquor. The cake was dried on the filter with applied vacuum for approx. 30 minutes. 714.9 mg of white powder was recovered. Then, further drying was conducted under vacuum (<5 mbar) at room temperature. 710 mg of powder was recovered after overnight drying. Yield: 70.6%

XRPD pattern corresponds to Form A (see FIG. 47). Under light microscopy, small crystals (needle/rod shape) were observed (see FIG. 38). Crystalline Form A of Example 24d is herein referred to as Sample 46.

### Example 24e: Preparation of Crystalline Form A of Compound of Formula 1

994.9 mg of amorphous form of the compound of Formula 1 was placed in a 25-mL reactor with a magnetic stirring bar in the EasyMax 102 device. 6 mL of 2-propanol was added to the powder. Stirring was started at 500 rpm, and most of the material dissolved but some sticky material formed. Temperature was increased to 30° C., and a cloudy solution was obtained. An additional 1 mL of 2-propanol was added and a clear solution was obtained (some particles were not dissolved). 8 mL of water was added with 0.5 mL/min; local precipitation was observed after each drop of water. Seeding with approx. 5 mg of Sample 42 was conducted after the addition of 1 mL of water; seed crystals did not dissolve. After the water addition, a solution was observed at 30° C. Cooling was started to 25° C. with 5 K/hour. After one hour of stirring at 25° C., the suspension was filtered over fritted glass filter (porosity 4). The reactor was rinsed with 3 mL of the mother liquor. The cake was dried on the filter with applied vacuum for approx. 30 minutes. 1.0197 g of white powder was recovered. Then, further drying was conducted under vacuum (<5 mbar) at room temperature.

887.4 mg of powder was recovered after overnight drying. Yield: 89.2%

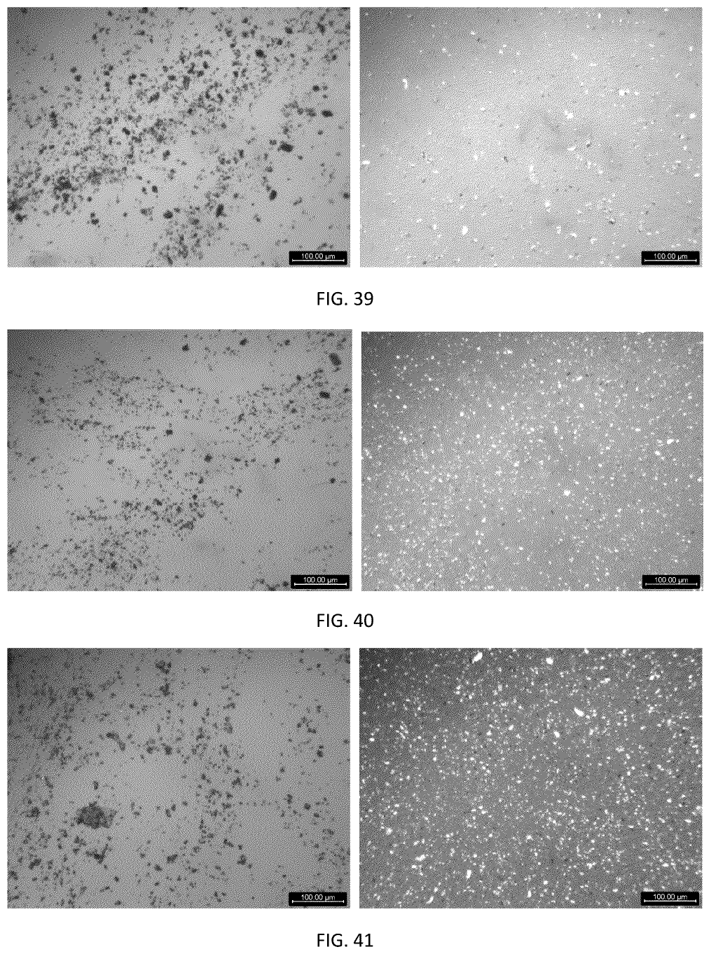

XRPD pattern corresponds to Form A (see FIG. 47). Under light microscopy, small crystals were observed; shape could not be determined (see FIG. 39). Crystalline Form A of Example 24e is herein referred to as Sample 47.

### Example 24f: Preparation of Crystalline Form A of Compound of Formula 1

1.002 g of amorphous form of the compound of Formula 1 was placed in a 25-mL reactor with a magnetic stirring bar in the EasyMax 102 device. 2 mL of 2-propanol was added to the powder, and a sticky material formed. Stirring was started at 500 rpm, and heating was initiated to 60° C. with 1 K/min. At room temperature, a cloudy solution was observed and at approx. 38° C., a suspension was obtained. A clear solution was observed at 60° C., and 3 mL of water was added with 0.1 mL/min. Stirring speed was increasing to 700 rpm. After the water addition, a fine suspension was obtained, and seeding was conducted with approx. 5 mg of Sample 42. Further stirring was conducted at 60° C. for 30 minutes and then cooling was started to 22° C. with 2K/hour. The suspension was then filtered over a fritted glass filter (porosity 4). The reactor was rinsed twice with 3 mL of the mother liquor. The cake was dried on the filter with applied vacuum for approx. 30 minutes. 1.3642 g of white powder was recovered. Then, further drying was conducted under vacuum (<5 mbar) at room temperature 912.5 mg of powder was recovered after overnight drying. Yield: 91%

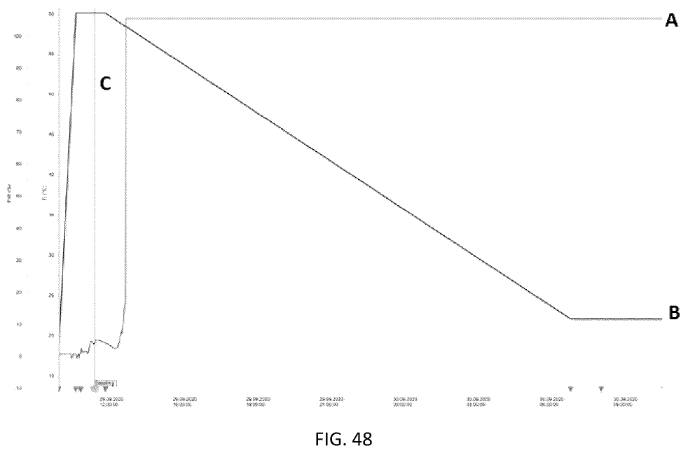

XRPD pattern corresponds to Form A (see FIG. 47). Under light microscopy, small crystals were observed; shape could not be determined (see FIG. 40). Crystalline Form A of Example 24f is herein referred to as Sample 48. FIG. 48 depicts this example: the temperature (blue curve) and the turbidity (Green curve) are shown as functions of time. The orange marker represents the seeding point.

### Example 24q: Preparation of Crystalline Form A of Compound of Formula 1

1.0147 g of amorphous form of the compound of Formula 1 was placed in a 25-mL reactor with a magnetic stirring bar in the EasyMax 102 device. 2 mL of 2-propanol was added to the powder and a sticky material formed. Stirring was started at 500 rpm, and heating was initiated to 60° C. with 1 K/min. At 60° C. a clear solution was observed and 4 mL of water was added with 0.1 mL/min. Stirring speed was increased to 700 rpm. After the water addition, a fine suspension was obtained, and seeding was conducted with approx. 5 mg of Sample 42. Further stirring was conducted at 60° C. for 30 minutes and then cooling was started to 22° C. with 2 K/hour. After overnight stirring, the reactor temperature was still at 60° C., the cooling program did not start. A suspension was already observed at 60° C. The cooling was started to 22° C. with 10 K/hour; then the suspension was further stirred at 22° C. for 1 h 30 minutes. The suspension was filtered over a fritted glass filter (porosity 4). The reactor was rinsed with 3 mL of the mother liquor. The cake was dried on the filter with applied vacuum for approx. 1 hour. 923.4 mg of white powder was recovered. Then, further drying was conducted under vacuum (<5 mbar) at room temperature 921.5 mg of powder was recovered after overnight drying. Yield: 91%

XRPD pattern corresponds to Form A (see FIG. 47). Under light microscopy, small crystals were observed; shape could not be determined (see FIG. 41). Crystalline Form A of Example 24g is herein referred to as Sample 49 (see FIG. 41).

### Example 24h: Preparation of Crystalline Form A of Compound of Formula 1

1.0088 g of amorphous form of the compound of Formula 1 was placed in a 25-mL reactor with a magnetic stirring bar in the EasyMax 102 device. 2 mL of 2-propanol was added to the powder, and a sticky material formed. Stirring was started at 500 rpm, and heating was initiated to 60° C. with 1 K/min. At 60° C. a clear solution was observed and 4 mL of water was added with 0.1 mL/min. Stirring speed was increased to 700 rpm. After the water addition, a fine suspension was obtained, and seeding was conducted with Sample 42 (approx. 5 mg). Further stirring was conducted at 60° C. for 1 h 30 minutes; the stirring speed was changed to 700 rpm. Cooling was started to 20° C. with 2 K/hour. Then temperature cycling was programmed over the weekend:
Wait 1 hour at 20° C.
Heat to 40° C. with 15 K/hour
Cool to 20° C. with 5 K/hour

These three steps were repeated 4 times. Then the suspension was stirred at 20° C. for one day with 1000 rpm. Filtration was conducted over a fritted glass filter (porosity 4). The reactor was rinsed with 3 mL of the mother liquor. The cake was dried on the filter with applied vacuum for approx. 30 minutes. 972.5 mg of white powder was recovered. Then, further drying was conducted under vacuum (<5 mbar) at room temperature.

927.8 mg of powder was recovered after overnight drying. Yield: 92%

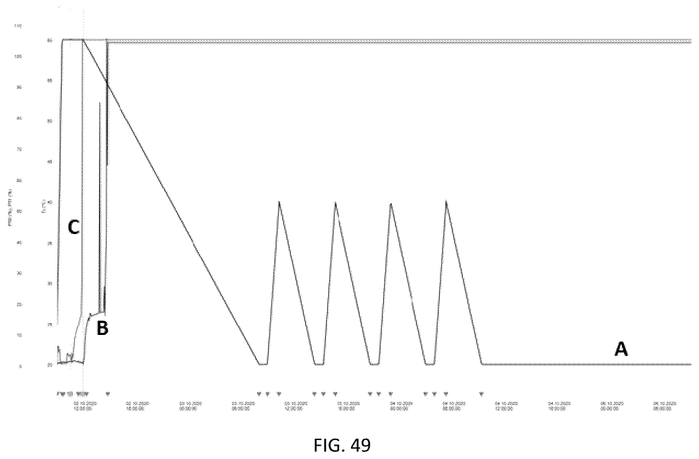

FIG. 49 depicts this example: the temperature (blue curve) and the turbidity (Green curve) are shown as functions of time. The gray markers represent the seeding point.

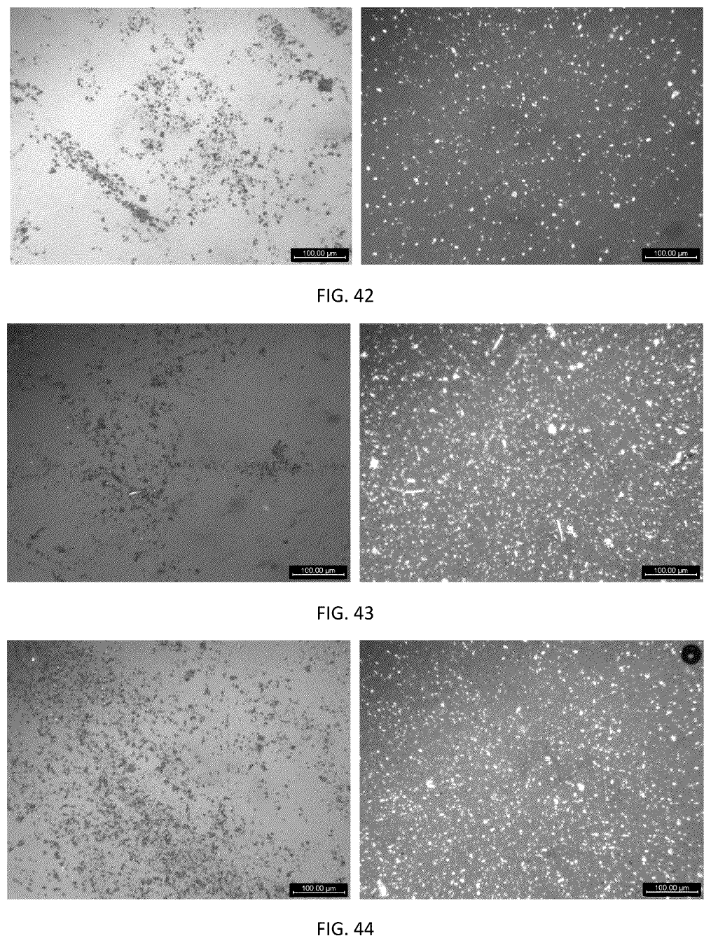

XRPD pattern corresponds to Form A (see FIG. 47). Under light microscopy, small crystals were observed; shape could not be determined (see FIG. 42). Crystalline Form A of Example 24h is herein referred to as Sample 50.

### Example 24i: Preparation of Crystalline Form A of Compound of Formula 1

1.0056 g of amorphous form of the compound of Formula 1 was placed in a 25-mL reactor with a magnetic stirring bar in the EasyMax 102 device. 2 mL of ethyl acetate was added to the powder, and a sticky material formed. Stirring was started at 500 rpm, and heating was initiated to 60° C. with 1 K/min. At 60° C. a clear solution was observed, and 8 mL of heptane was added with 0.1 mL/min. Stirring speed was increasing to 700 rpm. After the heptane addition, a clear solution was observed, and seeding was conducted with Sample 42 (approx. 5 mg). Seed crystals did not dissolve and cooling was started to 20° C. with 2K/hour. Then temperature cycling was programmed over the week-end:
Wait 1 hour at 20° C.
Heat to 40° C. with 15K/hour
Cool to 20° C. with 5K/hour

These three steps were repeated 4 times. Then the suspension was stirred at 20° C. for one day with 1000 rpm. Filtration was conducted over a fritted glass filter (porosity 4). The reactor was rinsed with 3 mL of the mother liquor. The cake was dried on the filter with applied vacuum for approx. 30 minutes. 902.7 mg of white powder was recovered. Then, further drying was conducted under vacuum (<5 mbar) at room temperature 900.9 mg of powder was recovered after overnight drying. Yield: 90%

FIG. 49 depicts this example: the temperature (blue curve) and the turbidity (Green curve) are shown as functions of time. The gray markers represent the seeding point.

XRPD pattern corresponds to Form A (see FIG. 47). Under light microscopy, small crystals (needle/rod shape) were observed (see FIG. 43). Crystalline Form A of Example 24i is herein referred to as Sample 51.

### Example 24j: Preparation of Crystalline Form A of Compound of Formula 1

1.0120 g of amorphous form of the compound of Formula 1 was placed in a 25-mL reactor with a magnetic stirring bar in the EasyMax 102 device. 2 mL of 2-propanol was added to the powder and, a sticky material formed. Stirring was started at 500 rpm, and heating was initiated to 60° C. with 1 K/min. At 60° C. a clear solution was observed, and 4 mL of water was added with 1 mL/min. Stirring speed was increased to 700 rpm. After the water addition, a suspension was obtained and seeding was conducted with Sample 42. The stirring speed was changed to 700 rpm, and cooling was started to 20° C. with 10 K/hour. After 2 days and 17 hours (65 hours in total) of stirring at 20° C., the suspension was filtered over a fritted glass filter (porosity 4). The reactor was rinsed with 4 mL of the mother liquor. The cake was dried on the filter with applied vacuum for approx. 1 hour. 956.3 mg of white powder was recovered. Then, further drying was conducted under vacuum (<5 mbar) at room temperature 938.6 mg of powder was recovered after overnight drying. Yield: 93%

XRPD pattern corresponds to Form A (see FIG. 47). Under light microscopy, small crystals were observed; shape could not be determined (see FIG. 44). Crystalline Form A of Example 24j is herein referred to as Sample 52.

### Example 24k: Preparation of Crystalline Form A of Compound of Formula 1

40.0370 g of amorphous form of the compound of Formula 1 was placed in a 400-mL reactor in the EasyMax 402 device with an anchor stirrer. 80 mL of 2-propanol was added to the slightly orange powder. Stirring was set to 100 rpm, and heating was initiated to 60° C. with 1 K/min. At 60° C. a clear orange solution was observed, and 160 mL of water was added with 1 mL/min. After the addition of approx. 90 mL of water, a cloudy solution was observed. A fine suspension was observed after the water addition. Further stirring was conducted at 60° C., and a suspension formed; seeding was conducted with Sample 46 (approx. 40 mg). The suspension was further stirred at 60° C. for 30 minutes, and the stirring speed was changed to 200 rpm. Then cooling was started to 20° C. with 2 K/hour. Temperature cycling was programmed over the weekend:
Wait 1 hour at 20° C.
Heat to 40° C. with 15 K/hour
Cool to 20° C. with 5 K/hour

These three steps were repeated 3 times. Then the suspension was stirred at 20° C. for one day with 200 rpm. Filtration was conducted over a fritted glass filter (porosity 4). The reactor was rinsed with 25 mL of the mother liquor, and the cake was washed with 30 mL of a 2-prOH/water 1:2 mixture. The cake was dried on the filter with applied vacuum for approx. 2 hours. 38.9163 g of white powder was recovered. Then, further drying was conducted under vacuum (<5 mbar) at room temperature 38.74 g of powder was recovered after overnight drying. Yield: 96.8%

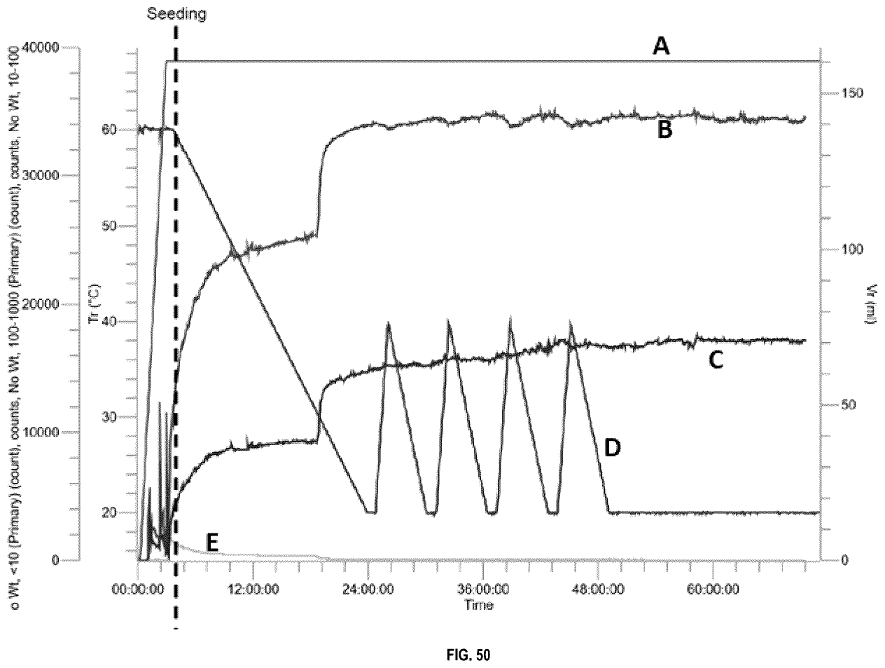

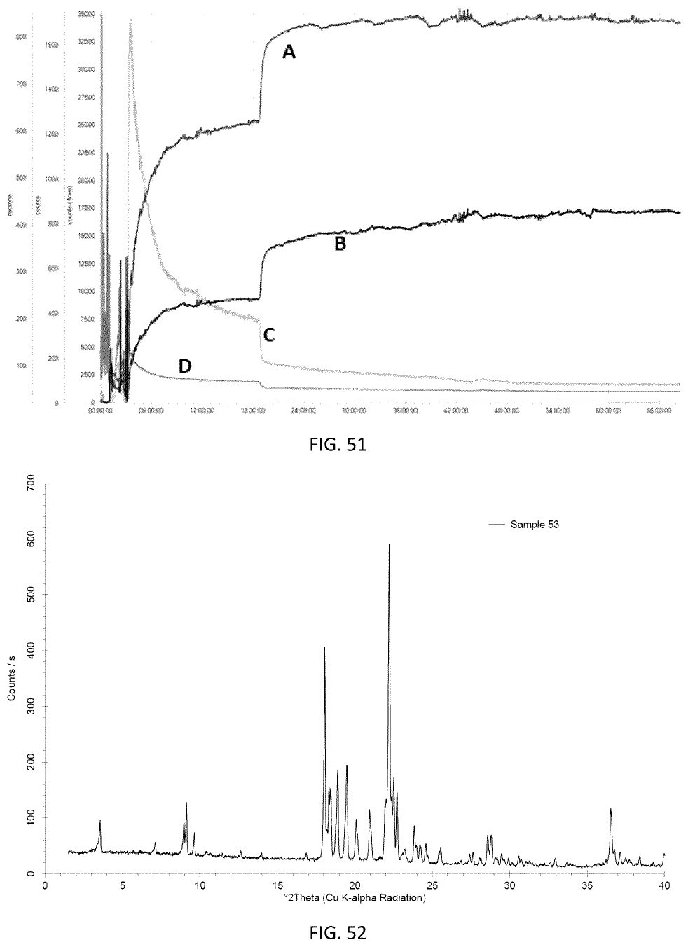

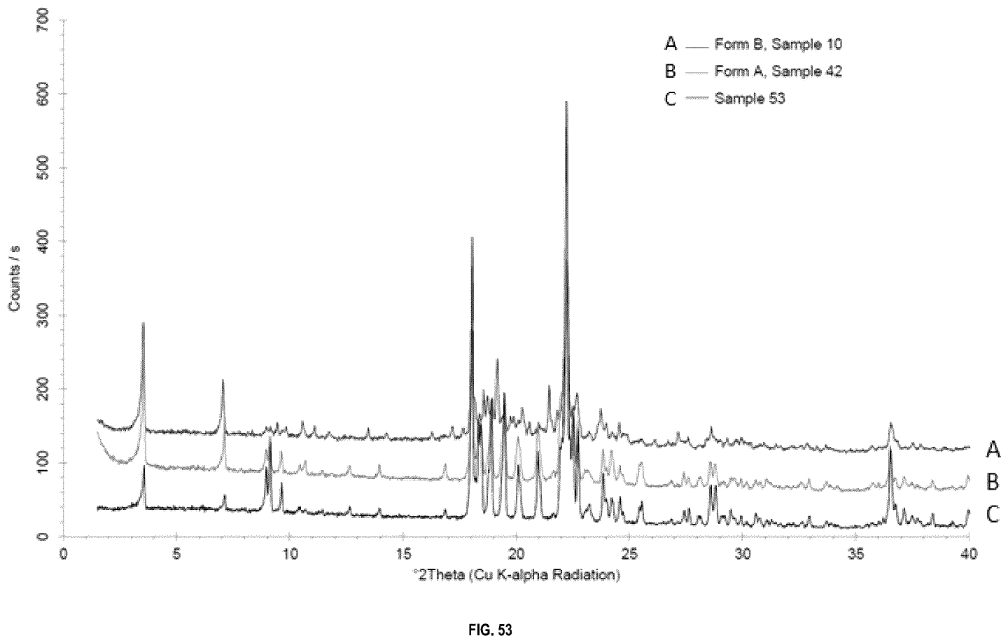

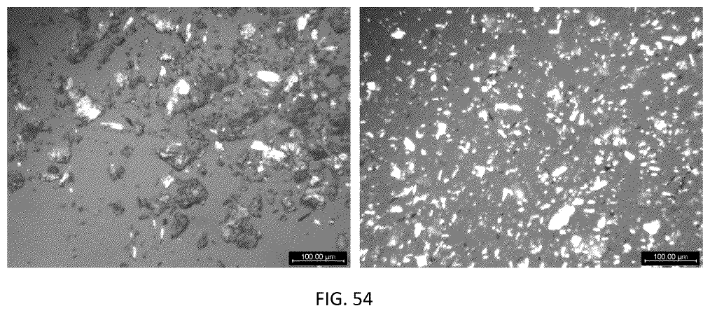

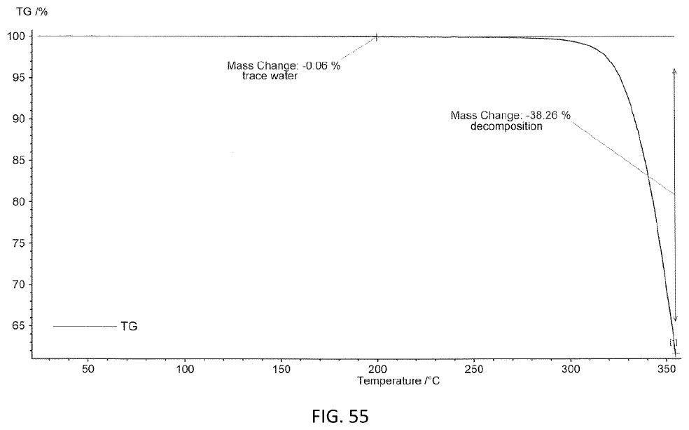

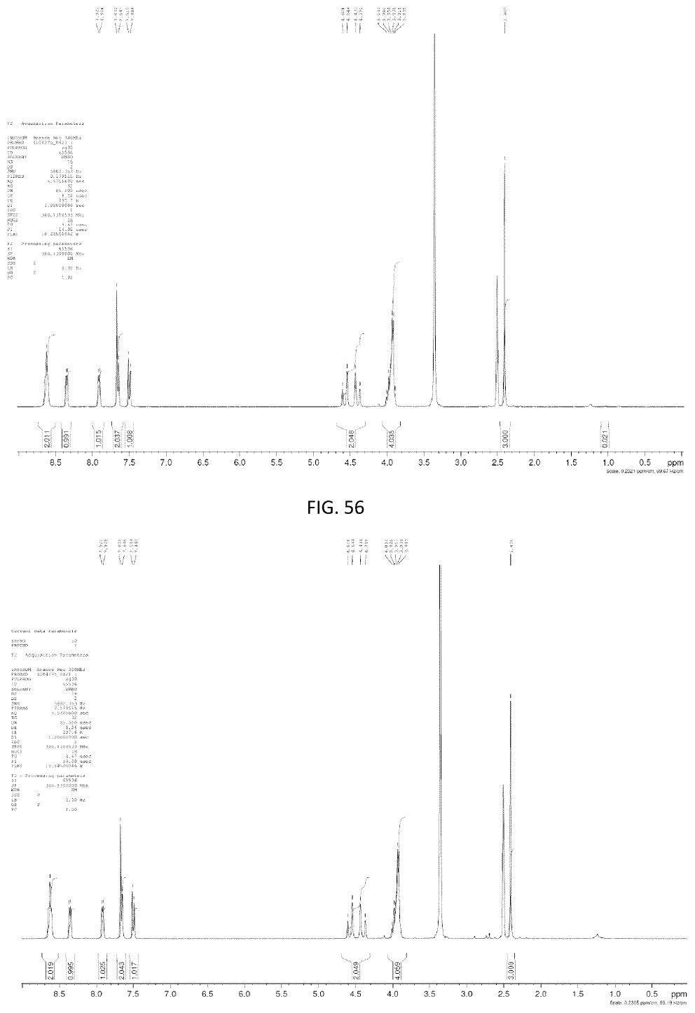

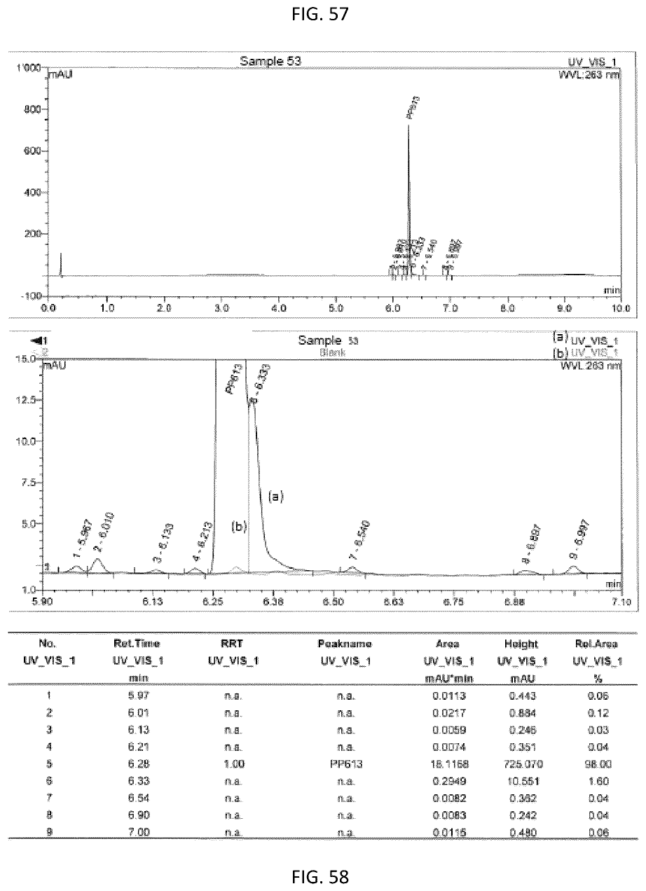

XRPD pattern corresponds to Form A (see FIGS. 52 and 53). Under light microscopy, small crystals were observed; shape could not be determined (see FIG. 54). ¹H-NMR analysis shows that the NMR spectrum is consistent with the amorphous form of the compound of Formula 1 but contains fewer impurities (see FIGS. 56 and 57). Traces of 2-propanol are visible. HPLC analysis shows a purity of 98 area % (see FIG. 58). Thermogravimetric analysis (TG-FTIR) indicated traces of water observed from 25 to 200° C. (see FIG. 55). Crystalline Form A of Example 24k is herein referred to as Sample 53. FIG. 50 shows a graphical representation of this example with temperature, water volume, and particle count curves as a function of time. FIG. 51 shows a graphical representation of this example with the mean square and particle count curves as a function of time.

### Example 24l: Preparation of Crystalline Form A of Compound of Formula 1

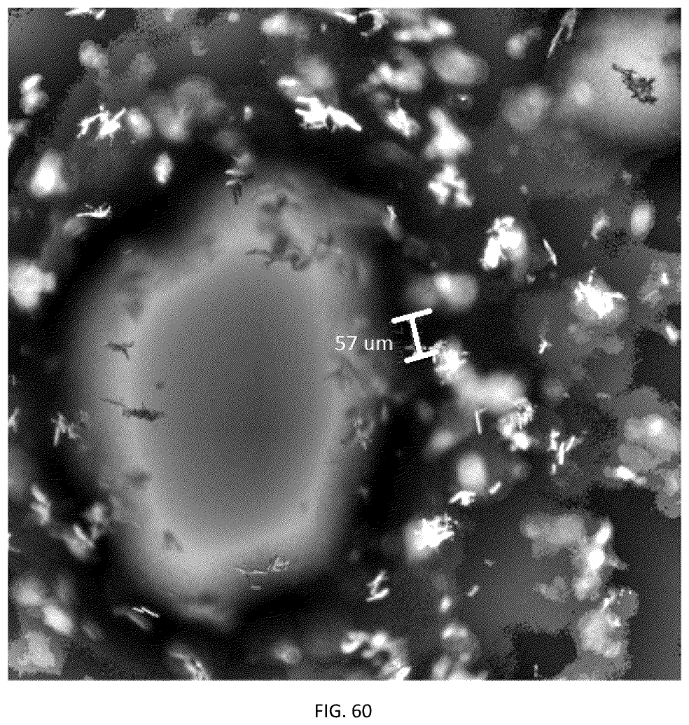

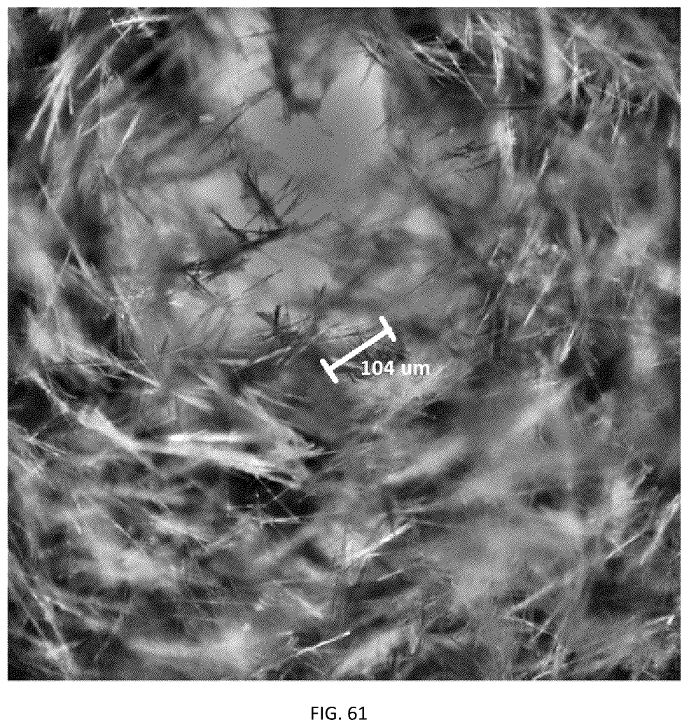

35.1697 g of amorphous form of the compound of Formula 1 was placed in a 400-mL reactor in the EasyMax 402 device with an anchor stirrer. 70 mL of ethyl acetate was added to the slightly orange powder. Stirring was set to 100 rpm, and heating was initiated to 60° C. with 1 K/min. At 60° C. a clear orange solution was observed, and 280 mL of heptane was added with 2 mL/min. After the addition, a clear solution was obtained, and seeding was conducted with sample 46 (approx. 40 mg). (FIG. 60 shows crystals just after seeding.) Seed crystals did not dissolve, and a suspension formed. The suspension was further stirred at 60° C. for 30 minutes, and the stirring speed was changed to 200 rpm. Then cooling was started to 20° C. with 2 K/hour. (FIG. 61 shows crystals after suspension formation when the cooling ramp was initiated.) Temperature cycling was programmed over the week-end:
Wait 1 hour at 20° C.
Heat to 40° C. with 15 K/hour
Cool to 20° C. with 5 K/hour

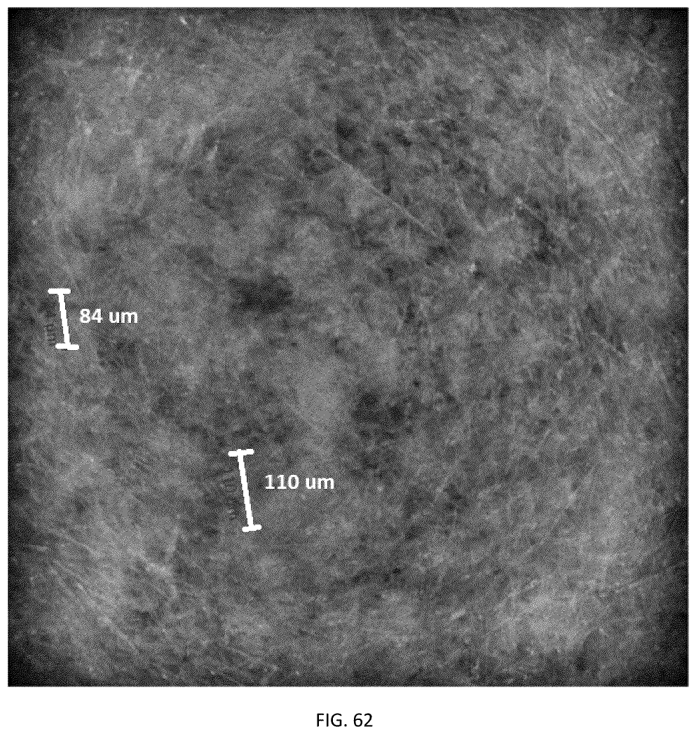

These three steps were repeated 3 times. (FIG. 62 shows the suspension at the end of the process.) Then the suspension was stirred at 20° C. for one day with 200 rpm. Filtration was conducted over a fritted glass filter (porosity 4). The reactor was rinsed with 25 mL of the mother liquor, and the cake was washed with 30 mL of an ethyl acetate/heptane 1:4 mixture. The cake was dried on the filter with applied vacuum for approx. 2 hours. 30.8252 g of white powder was recovered. Then, further drying was conducted under vacuum (<5 mbar) at room temperature 30.7783 g of powder was recovered after overnight drying. Yield: 87.5%

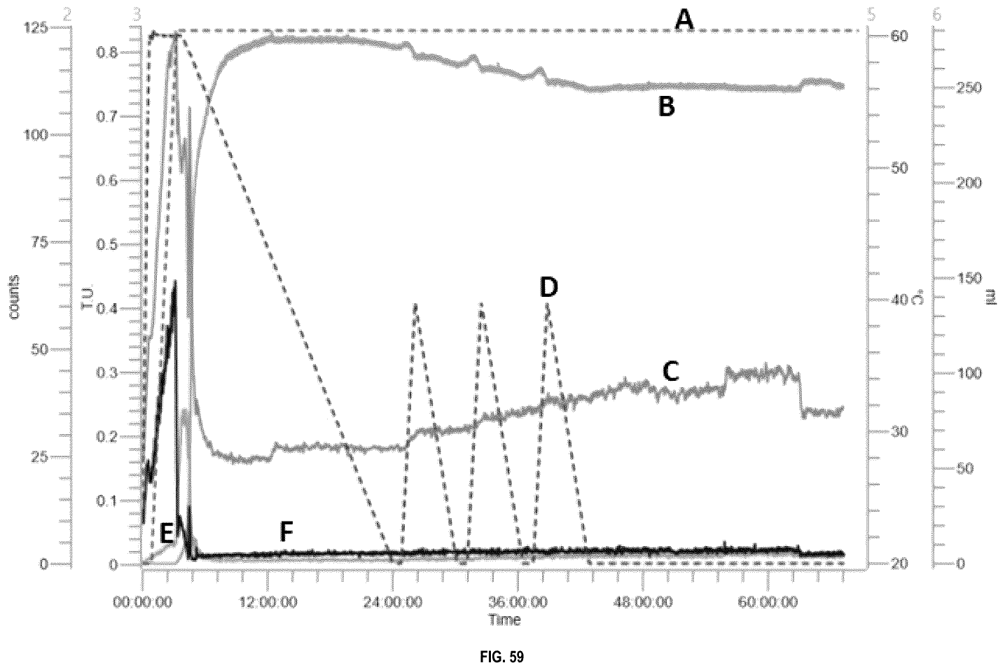

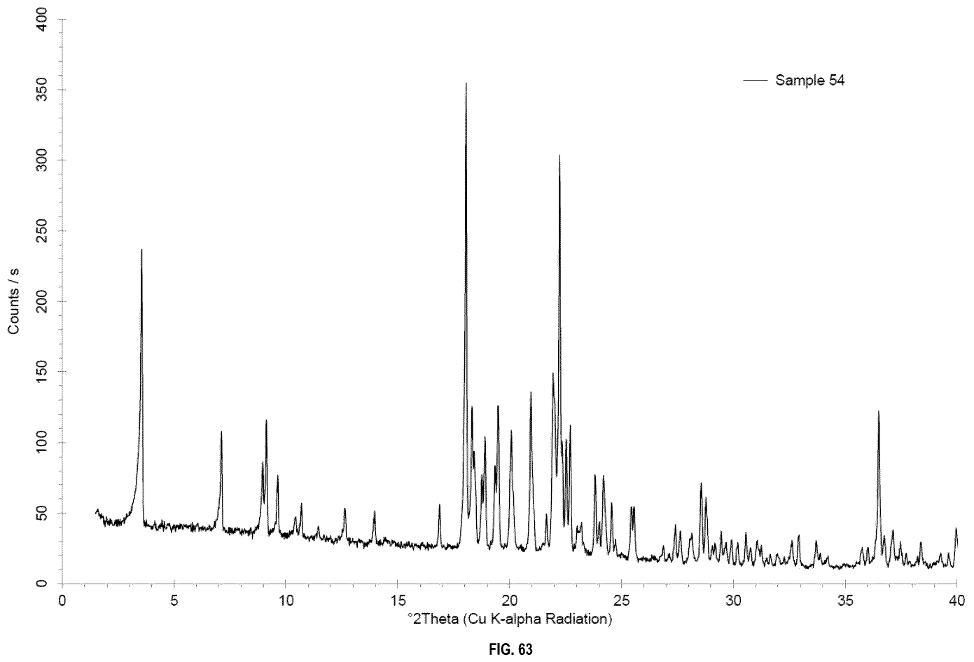

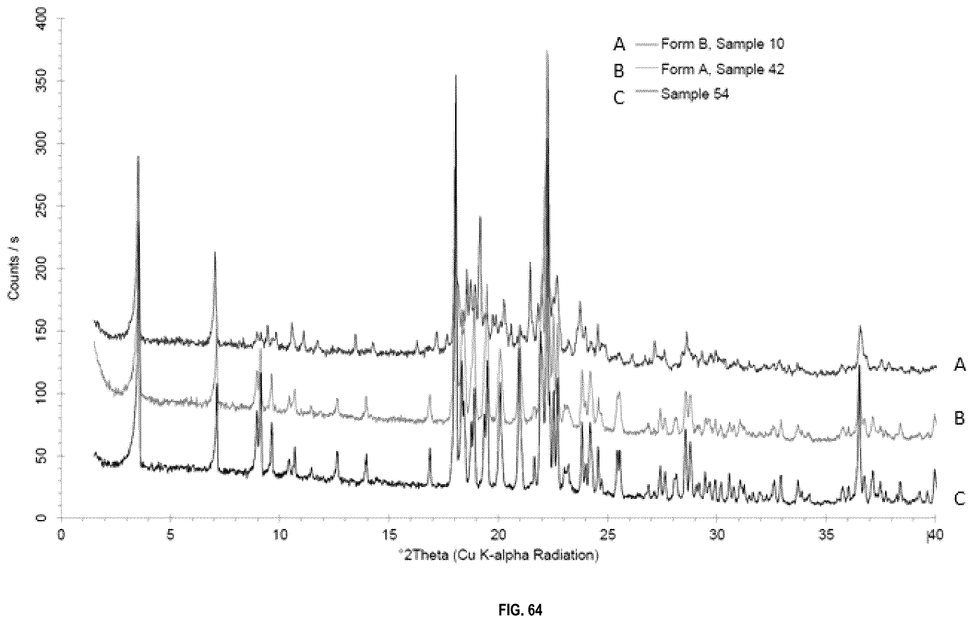

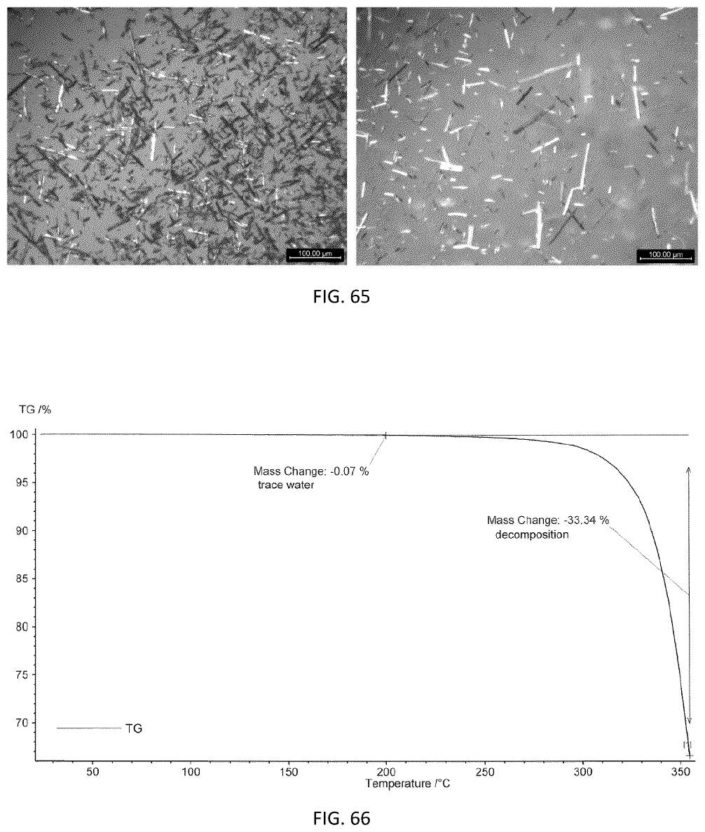

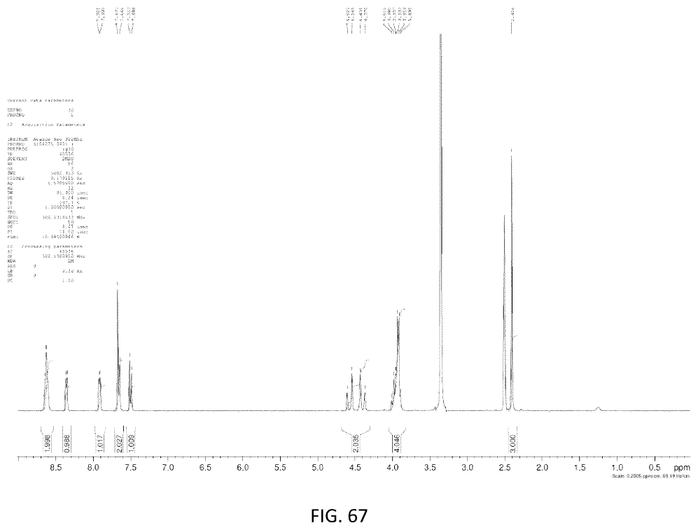

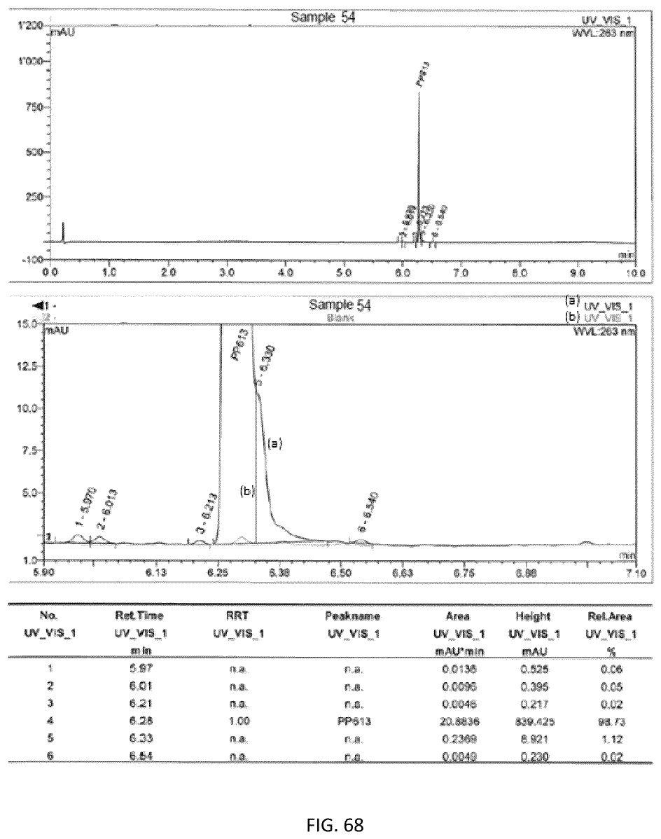

XRPD pattern corresponds to Form A (see FIGS. 63 and 64). Under light microscopy, small crystals (needle/rod shape) were observed (see FIG. 65). ¹H-NMR analysis shows that the NMR spectrum is consistent with the amorphous form of the compound of Formula 1 but contains fewer impurities (see FIG. 67). HPLC analysis shows a purity of 98.7 area % (see FIG. 68). Thermogravimetric analysis (TG-FTIR) indicated traces of water observed from 25 to 200° C. (see FIG. 66). Crystalline Form A of Example 24l is herein referred to as Sample 54. FIG. 59 shows a graphical representation of this example with temperature, added volume, turbidity, and particle count curves as a function of time.

### Example 25a: Preparation of Crystalline Form B of Compound of Formula 1

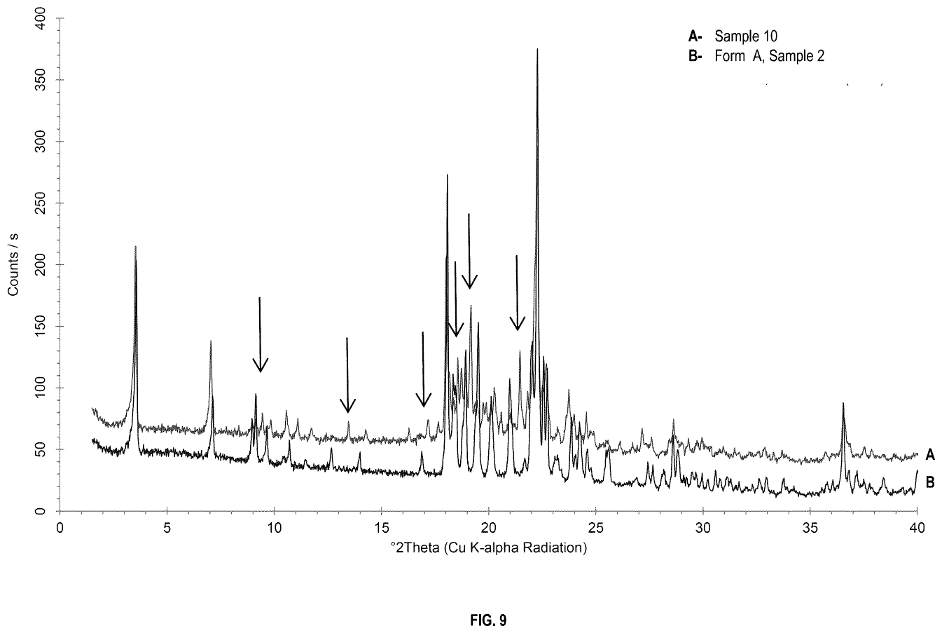

83.5 mg of amorphous form of the compound of Formula 1 was dissolved in 500 μL of methanol at room temperature. The vial was opened to allow solvent evaporation at room temperature. After three days, a glass residue was obtained and further evaporated under nitrogen flow. After 5 hours, the glassy residue was removed with a spatula and submitted for XRPD analysis (see FIGS. 8, 9, 24, 46, 47, 53, and 64). Crystalline Form B of Example 25a is herein referred to as Sample 10. ¹H-NMR analysis showed that the NMR spectrum of Sample 10 is consistent with the NMR spectrum of the amorphous form of the compound of Formula 1; residual 2-propanol (0.014 eq) and residual methanol (0.5 eq) were visible. DVS analysis indicated absorption of 1.2% water at 95% relative humidity for five hours (see FIGS. 22 and 23).

### Example 25b: Preparation of Crystalline Form B of Compound of Formula 1

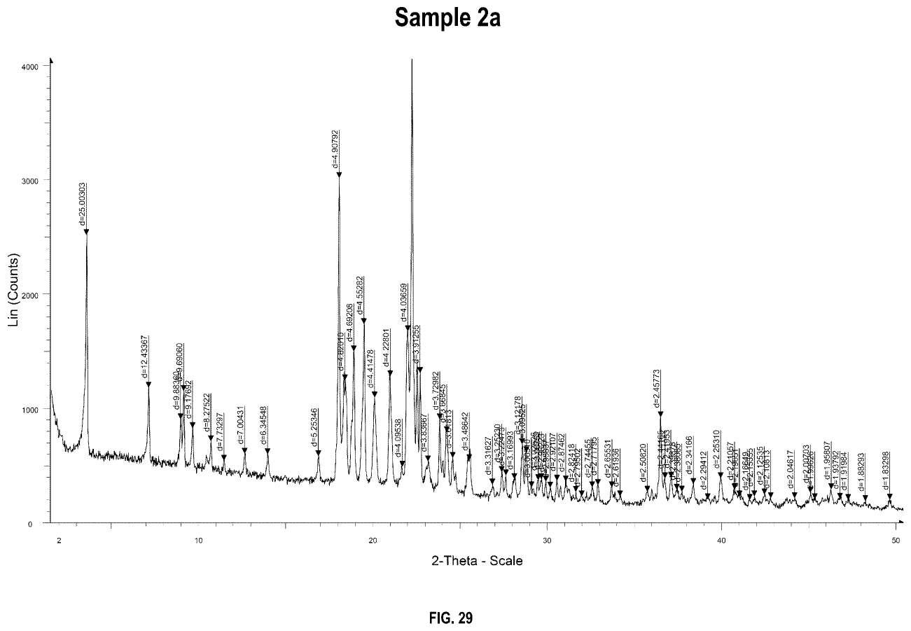

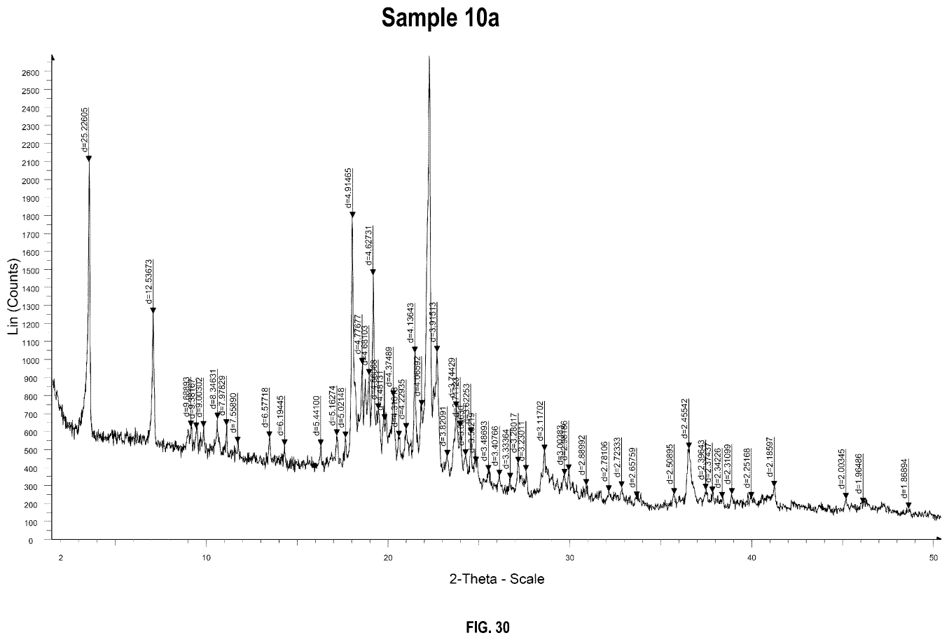

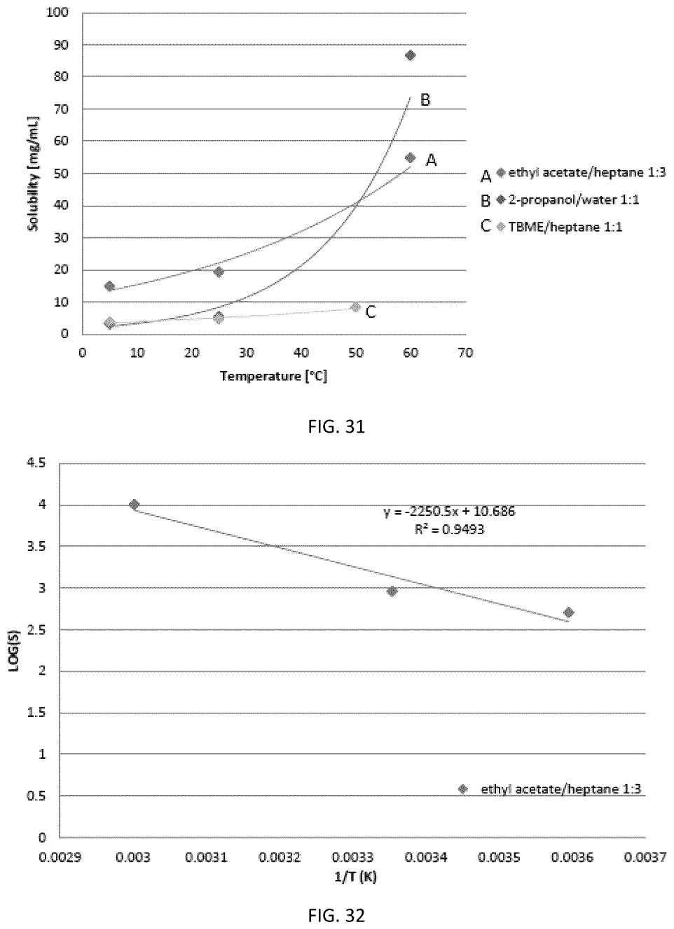

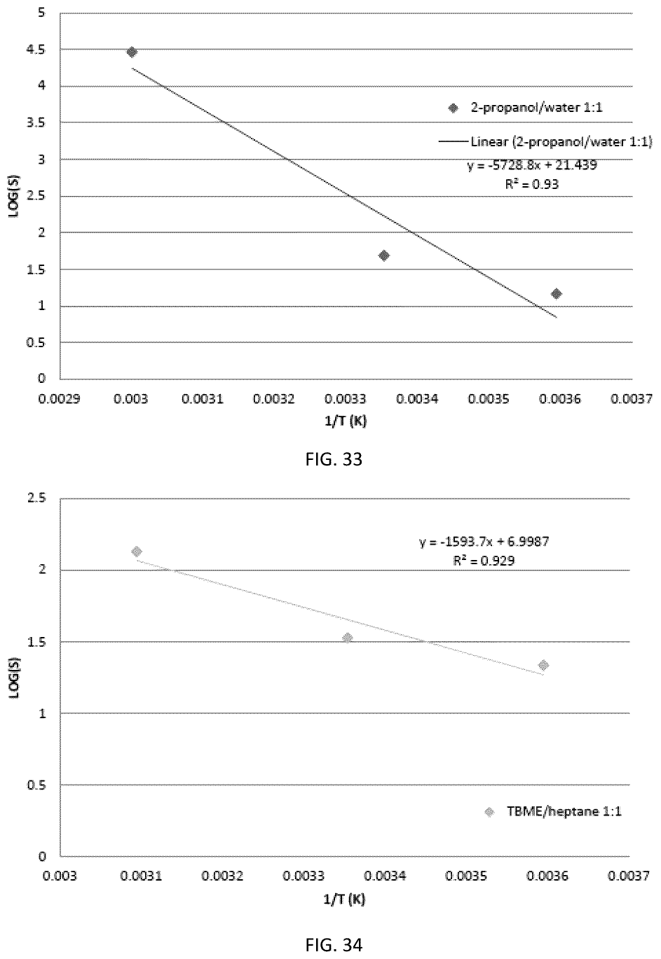

From Example 25a, the remaining Sample 10 was dried overnight at room temperature and 5 mbar. Crystalline Form B of Example 3b is herein referred to as Sample 10a. The XRPD pattern of Sample 10a corresponded to the XRPD pattern of Sample 10 (see FIGS. 19, 24, 30). Thermogravimetric analysis (TG-FTIR) indicated weight loss of 0.80% of water from 25 to 250° C. (see FIG. 11). DSC analysis indicated glass transition at 59° C. with ΔCₚ step of 0.2 J/(g-° C.), melting at 132.5° C., onset of 124° C. with enthalpy of fusion of 45.7 J/g (see FIG. 20).

### Example 26a: Preparation of Crystalline Form C of Compound of Formula 1

1 gram of the amorphous form of the compound of Formula 1 was dissolved in ethyl acetate/n-heptane (16V, 1/3 v/v) at 60° C., then cooled to 50° C. over 1 hour. After holding for 18 hours, a small aliquot was taken, filtered, and subjected to XRPD analysis (see FIGS. 69, 70, 75, 76). After confirmation that Form C is obtained, the batch was concentrated to 1 V at 45° C. and exchanged with heptane (5v×2) to a final volume of 5 V, then filtered at 20-25° C., and dried in an oven at 45° C. for 18 hours. DSC analysis indicated a melting peak at 142.8° C., onset of 139.7° C., with enthalpy of fusion of 58.3 J/g (see FIG. 71).

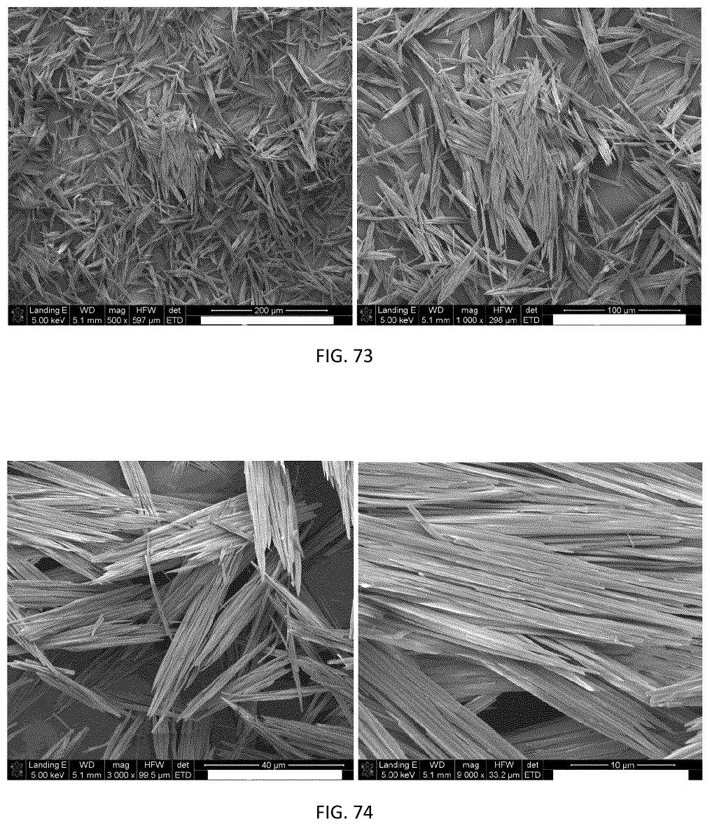

Scanning electron microscopy (SEM) was carried out on Crystalline Form C of the compound of Formula 1. Six different magnifications were used, namely 100×, 250×, 500×, 1000×, 3000×, and 9000×. The SEM images at 100× and 250× are shown in FIG. 72; the SEM images at 500× and 1000× are shown in FIG. 73; and the SEM images at 3000× and 9000× are shown in FIG. 74.

Crystalline Form C of Example 26a is herein referred to as Sample 55.

### Example 26b: Preparation of Crystalline Form C of Compound of Formula 1

1 gram of the amorphous form of the compound of Formula 1 was dissolved in 16 mL of an ethyl acetate/heptane 1:3 (v/v) mixture at 60° C. A solution was obtained, and the temperature was decreased to 50° C. in one hour. After overnight stirring (approximately 18 hours), a suspension formed and 1.5 mL of the suspension was filtered and subjected to XRPD analysis. Form C was obtained.

The temperature was then decreased to 45° C. and the reactor was opened to allow solvent evaporation. After overnight stirring, only 3 mL had evaporated, so further evaporation was conducted with nitrogen flow. After 4 hours, the remaining solvent was approximately 5 volumes, and 10 mL of heptane was slowly added. The obtained suspension was stirred for 1 hour at 45° C. Then, evaporation was started with weak nitrogen flow at 45° C. After overnight stirring, approximately 8 volumes of suspension remained. The suspension was filtered over fritted glass (porosity 4). The cake was dried on the filter with applied vacuum. The powder was subjected to XRPD analysis, and Form C was obtained.

Crystalline Form C of Example 26b is herein referred to as Sample 60.

### Example 27a: Competitive Slurry Equilibration Experiment

53 mg of Sample 42 (Form A) and 47 mg of Sample (Form C) were suspended in 1 mL of an ethyl acetate/heptane (1:3) mixture at room temperature. After two hours of stirring at room temperature, one additional mL of the solvent mixture was added to the suspension. After another 1 hour of stirring, the suspension was seeded with Sample 42 and Sample 55 (about 10-20 mg). Further stirring was conducted at room temperature for one week. Then the suspension was filtered using a centrifugal unit filter (PTFE, 0.2 μm, 5000 rpm, room temperature). The recovered filter cake was submitted for XRPD analysis, which showed a pattern corresponding to a mixture of Forms A and C. This resulting mixture of Crystalline Forms A and C is herein referred to as Sample 56.

The remaining substance from Sample 56 (approx. 42 mg) was further equilibrated in 0.5 mL of an ethyl acetate/heptane (1:3) mixture at room temperature After one month of stirring, the suspension was filtered using a centrifugal unit filter (PTFE, 0.2 μm, 5000 rpm, room temperature). The recovered filter cake was submitted for XRPD analysis, which showed a pattern corresponding to Form C. The resulting Crystalline Form C of Example 27a is herein referred to as Sample 56A.

### Example 27b: Competitive Slurry Equilibration Experiment

71 mg of Sample 42 (Form A) and 73 mg of Sample 55 (Form C) were suspended in 1 mL of an ethyl acetate/heptane (1:3) mixture at 60° C. After two hours of stirring at 60° C., one additional mL of the solvent mixture was added to the suspension. After another 1 hour of stirring, the suspension was seeded with Samples 42 and 55 (about 10-20 mg). Further stirring was conducted at 60° C. After four days of stirring, a solution with material on the glass side was obtained. Approx. 25 mg of Sample 42 and 25 mg of Sample 55 were added and a suspension was obtained. After additional 3 days stirring at 60° C., the suspension was filtered using a centrifugal unit filter (PTFE, 0.2 μm, 5000 rpm, 40° C.). The recovered filter cake was submitted for XRPD analysis, which showed a pattern corresponding to Form C.

Crystalline Form C of Example 27b is herein referred to as Sample 57.

### Example 27c: Competitive Slurry Equilibration Experiment

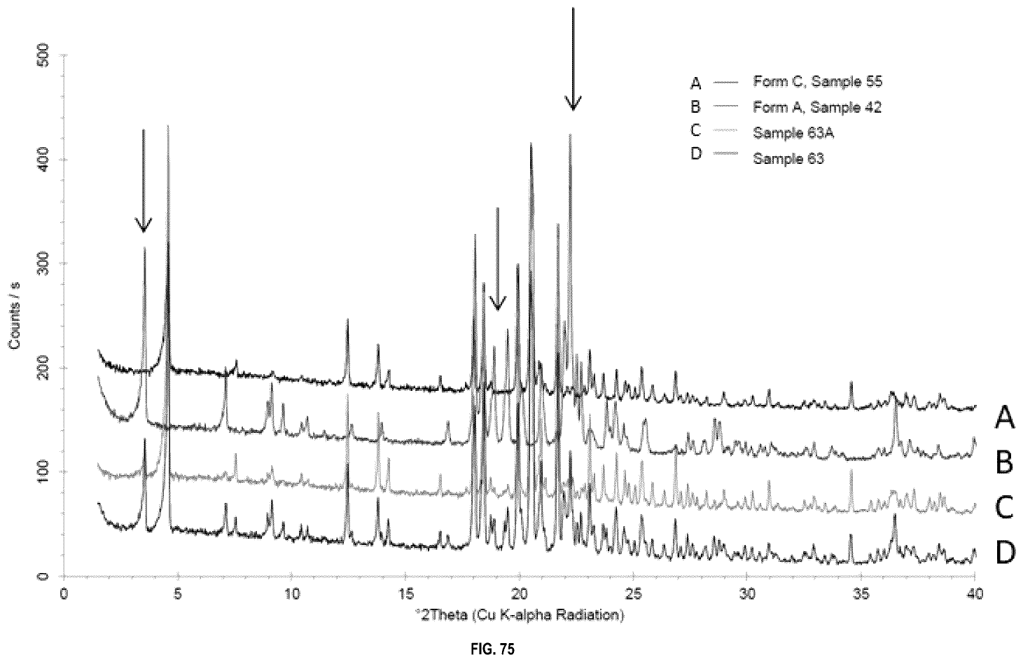

111 mg of Sample 42 (Form A) and 121 mg of Sample 55 (Form C) were suspended in 2 mL of an ethyl acetate/heptane 1:3 mixture at 30° C. After 30 minutes of stirring, additional 2 mL of the solvent mixture was added. After one hour of stirring at 30° C., the suspension was seeded with Sample 42 and Sample 55 (approx. 10 mg of each sample). Further stirring was conducted at 30° C. for two weeks. Then the half of the suspension was filtered using a centrifugal unit filter (PTFE, 0.2 μm, 5000 rpm, 30° C.). The recovered filter cake was designated Sample 63 and was submitted for XRPD analysis. The XRPD pattern corresponded to a mixture of Crystalline Forms A and C (see FIG. 75).

The other half of the suspension was further stirred for four additional weeks (total stirring time of 6 weeks). During the equilibration, some material was again observed on the wall of the vial (just above the suspension); therefore, the mixture was submitted to vortex treatment twice a day in order to bring all the material back into suspension. Then the suspension was filtered using a centrifugal unit filter (PTFE, 0.2 μm, 5000 rpm, 30° C.). The recovered filter cake was designated Sample 63A and was submitted for XRPD analysis. The XRPD pattern corresponded to Form C with a small amount of Form A (see FIG. 75).

### Example 27d: Competitive Slurry Equilibration Experiment

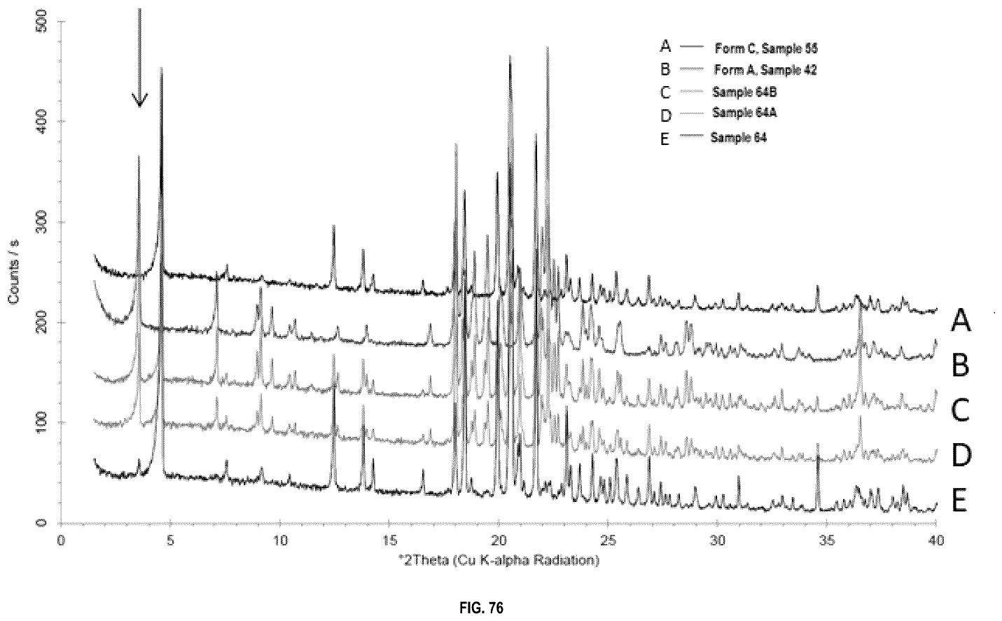

104 mg of Sample 42 (Form A) and 105 mg of Sample 55 (Form C) were suspended in 2 mL of an ethyl acetate/heptane 1:3 mixture at 35° C. After 30 minutes of stirring, additional 2 mL of the solvent mixture was added. After one hour of stirring at 35° C., the suspension was seeded with Sample 42 and Sample 55 (approx. 10 mg of each sample). Further stirring was conducted at 35° C. for two weeks. Then the half of the suspension was filtered using a centrifugal unit filter (PTFE, 0.2 μm, 5000 rpm, 35° C.). The recovered filter cake was designated Sample 64 and was submitted for XRPD analysis. The XRPD pattern corresponded to Form C with a small amount of Form A (see FIG. 76).

The rest of the suspension was further stirred for two weeks (total stirring time of 1 month). The suspension was filtered using a centrifugal unit filter (PTFE, 0.2 μm, 5000 rpm, 35° C.). The recovered filter cake was designated Sample 64A and was submitted for XRPD analysis. The XRPD pattern corresponded to a mixture of Forms A and C; the amount of Form A increased compared to Sample 64 (see FIG. 76).

The filter cake recovered in Sample 64A was further slurried in the recovered mother liquor at 35° C. Further stirring was conducted for two weeks (total stirring time of 6 weeks). During the equilibration, some material was again observed on the wall of the vial (just above the suspension); therefore, the mixture was submitted to vortex treatment twice a day in order to bring all the material back into suspension. The suspension was filtered using a centrifugal unit filter (PTFE, 0.2 μm, 5000 rpm, 35° C.). The recovered filter cake was designated Sample 64B and was submitted for XRPD analysis (see FIG. 76).

### Example 27e: Competitive Slurry Equilibration Experiments—Forms A and B

Competitive slurry equilibration experiments were carried out on Crystalline Forms A and B of the compound of Formula 1. Given the high solubility of the amorphous starting material in common organic solvents, the competitive slurry experiments were started with suspension of Form A, which were then seeded with Form B or with mixtures of Form A and Form B. Therefore mixtures with water, heptane, and cyclohexane were selected in order to decrease the solubility of the amorphous compound of Formula 1 in pure organic solvent and to obtain a suspension of Form A.

In all of the conducted experiments, Form A was obtained at the end of the equilibration time and Form B was no longer observed in the XRPD patterns. Thus, Form A is more stable form of Forms A and B.

TABLE 3

| Solvent | Conditions | Results |
|---|---|---|
| Ethanol/water 1:9 | 3 days at room temperature, Form A | Form A |
| (v/v) | suspension seeded with Form B | |
| 2-propanol/water 1:9 | 5 days at 75° C., Form A suspension | Form A |
| (v/v) | seeded with Form B | |
| Acetone/heptane 1:9 | 5 days at room temperature, Form A | Form A |
| (v/v) | suspension seeded with Form B | |
| Toluene/cyclohexane | 9 days at room temperature, suspension | Form A |
| 1:2 (v/v) | of Form A and Form B | |
| Ethyl acetate/heptane | 7 days at room temperature, suspension | Form A |
| 1:1 | of Form A and Form B | |

### Example 27f: Competitive Slurry Equilibration Experiments—Forms A and C

Competitive slurry equilibration experiments were carried out on Crystalline Forms A and C of the compound of Formula 1.

TABLE 4

| Solvent | Conditions | Results |
|---|---|---|
| Ethyl acetate/heptane 1:3 (v/v) | 1 week, room | Form A + Form C |
| | temperature | |
| Ethyl acetate/heptane 1:3 (v/V) | 5 weeks, room | Form A |
| | temperature | |
| Ethyl acetate/heptane 1:3 (v/v) | 1 week, 40° C. | Form A + Form C |
| Ethyl acetate/heptane 1:3 (v/v) | 4 weeks, 40° C. | Form C |
| Ethyl acetate/heptane 1:3 (v/v) | 1 week, 60° C. | Form C |
| 2-propanol/water 2:1 (v/v) | 1 week, room | Form A + Form C |
| | temperature | |
| 2-propanol/water 2:1 (v/v) | 3 weeks, room | Form A + small |
| | temperature | amount of Form C |
| 2-propanol/water 2:1 (v/v) | 1 week, 50° C. | Form C |
| Ethyl acetate/heptane 1:3 (v/v) | 2 weeks, 30° C. | Form A + Form C |
| Ethyl acetate/heptane 1:3 (v/v) | 6 weeks, 30° C. | Form C + small |
| | | amount of Form A |
| Ethyl acetate/heptane 1:3 (v/v) | 2 weeks, 35° C. | Form C + small |
| | | amount of Form A |
| Ethyl acetate/heptane 1:3 (v/v) | 4 weeks, 35° C. | Form A + Form C |
| Ethyl acetate/heptane 1:3 (v/v) | 6 weeks, 35° C. | Form A + Form C |

Those of ordinary skill in the art will recognize that the present invention may be embodied in other specific forms without departing from its spirit or essential characteristics. The described embodiments are to be considered in all respects only as illustrative and not restrictive. The scope of the present invention is, therefore, indicated by the appended claims rather than by the foregoing description. All changes which come within the meaning and range of equivalency of the claims are to be embraced within the scope of the present invention.

1. A crystalline form of a compound of Formula 1, 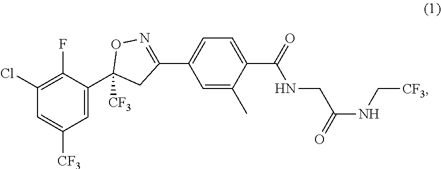 [SMILES: Cc1cc(C2=NO[C@@](c3cc(C(F)(F)F)cc(Cl)c3F)(C(F)(F)F)C2)ccc1C(=O)NCC(=O)NCC(F)(F)F]
exhibiting an X-ray powder diffraction (XRPD) pattern comprising peaks at 2θ±0.2° values of 18.1°, 19.5°, and 22.3°.

2. The crystalline form of claim 1, wherein the crystalline form is substantially pure.

3. A pharmaceutical composition comprising the crystalline form according to claim 1 as an active ingredient and at least one pharmaceutically acceptable carrier or diluent.

4. A method of preparing the crystalline form according to claim 1, the method comprising:
dissolving, optionally with stirring and/or heating, the compound of Formula 1 in one or more components A, wherein the compound of Formula 1 dissolved is an amorphous form, one or more crystalline forms, or a combination thereof, and wherein component A is an organic solvent suitable for dissolving the compound of Formula 1, optionally one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; optionally one or more of C₁-C₄ alcohol, acetone, acetonitrile, aniline, anisole, benzyl alcohol, butyronitrile, chloroform, DMSO, ethanol ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine; and optionally adding one or more components B, wherein component B is an antisolvent that reduces the solubility of the mixture, wherein component B comprises one or more of water and C₅-C₁₂ cyclic or acyclic hydrocarbon alkanes optionally (cyclohexane, hexane, heptane, n-heptane, octane, nonane, and decane, optionally one or more of water and heptane.

5. A method of preparing the crystalline form according to any claim 1, the method comprising:
dissolving, optionally with stirring and/or heating, the compound of Formula 1 in one or more components A, wherein the compound of Formula 1 dissolved is an amorphous form, one or more crystalline forms, a combination thereof, and wherein component A is an organic solvent suitable for dissolving the compound of Formula 1, optionally one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; optionally one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, medium-chain triglycerides, NMP, 1-octanol, 1-propanol, 2-propanol, TBME, THF, toluene, and triethylamine;
adding one or more components B, wherein component B is an antisolvent that reduces the solubility of the mixture, component B comprises one or more of water and C₅-C₁₂ cyclic or acyclic hydrocarbon alkanes optionally (cyclohexane, hexane, heptane, n-heptane, octane, nonane, and decane, optionally one or more of water and heptane, and filtering, washing, and drying the resulting solid.

6. A crystalline form of the compound of Formula 1 prepared by the method according to claim 4.

7. A crystalline form of a compound of Formula 1, 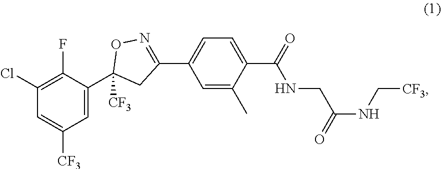 [SMILES: Cc1cc(C2=NO[C@@](c3cc(C(F)(F)F)cc(Cl)c3F)(C(F)(F)F)C2)ccc1C(=O)NCC(=O)NCC(F)(F)F]
exhibiting an X-ray powder diffraction (XRPD) pattern comprising peaks at 2θ±0.2° values of 4.6°, 20.5°, and 21.7°.

8. The crystalline form of claim 7, wherein the crystalline form is substantially pure.

9. A pharmaceutical composition comprising the crystalline form according to claim 7 as an active ingredient and at least one pharmaceutically acceptable carrier or diluent.

10. The pharmaceutical composition of claim 3, wherein the crystalline form makes up 80% or more of a total amount of the compound of Formula 1 in the pharmaceutical composition.

11. The pharmaceutical composition of claim 3, wherein the pharmaceutical composition is for treating pests in animals, optionally cats and/or dogs.

12. The pharmaceutical composition of claim 3, wherein said pests comprise ticks and/or fleas.

13. A method of preparing the crystalline form according to claim 7, the method comprising:
dissolving, optionally with stirring and/or heating, the compound of Formula 1 in one or more components A, wherein the compound of Formula 1 dissolved is an amorphous form, one or more crystalline forms, or a combination thereof, and wherein component A is an organic solvent suitable for dissolving the compound of Formula 1, optionally one or more of C₁-C₄ alcohol, acetone, acetonitrile, aniline, anisole, benzyl alcohol, butyronitrile, chloroform, cyclohexane, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; more optionally one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine; adding one or more components B, wherein component B is an antisolvent that reduces the solubility of the mixture, wherein component B comprises one or more of water and C₅-C₁₂ cyclic or acyclic hydrocarbon alkanes optionally (cyclohexane, hexane, heptane, n-heptane, octane, nonane, and decane, optionally one or more of water and heptane; evaporating the one or more components A and the one or more components B; and filtering the resulting solid.

14. A method of preparing the crystalline form according to claim 7, the method comprising:
dissolving the compound of Formula 1 in one or more components A, wherein the compound of Formula 1 dissolved is an amorphous form, one or more crystalline forms, or a combination thereof, and wherein component A is an organic solvent suitable for dissolving the compound of Formula 1, preferably one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, heptane, n-heptane, isobutyl acetate, MEK, methanol, medium-chain triglycerides, NMP, 1-octanol, 1-propanol, 2-propanol, TBME, THF, toluene, and triethylamine; more preferably one or more of C₁-C₄ alcohol, acetone, acetonitrile, aniline, anisole, benzyl alcohol, butyronitrile, chloroform, DMSO, ethanol, ethyl acetate, isopropyl acetate, ethyl lactate, isobutyl acetate, MEK, methanol, medium-chain triglycerides, NMP, 1-octanol, 1-propanol, 2-propanol, TBME, THF, toluene, and triethylamine; adding one or more components B, wherein component B comprises one or more of water and C₅-C₁₂ cyclic or acyclic hydrocarbon alkanes optionally (cyclohexane, hexane, heptane, n-heptane, octane, nonane, and decane, optionally one or more of water and heptane; stirring a solution formed from the dissolving step; evaporating the one or more components A and the one or more components B from the solution; adding one or more additional component B;
evaporating the one or more components A and the one or more components B from the solution containing one or more additional components B.

15. A crystalline form of the compound of Formula 1 prepared by the method according to claim 13.
